SEQUENCE LISTING (35 sequences)

SEQ ID NO: 1 - DNA, 20 bp, Artificial sequence
  Synthetic construct
  misc_feature: Gene BAD
  misc_feature: Forward primer sequence
cccagagtttgagccgagtg

SEQ ID NO: 2 - DNA, 19 bp, Artificial sequence
  Synthetic construct
  misc_feature: Gene BAD
  misc_feature: Reverse primer sequence
cccatcccttcgtcgtcct

SEQ ID NO: 3 - DNA, 21 bp, Artificial sequence
  Synthetic construct
  misc_feature: Forward primer sequence
  misc_feature: Gene BAX
cccgagaggtctttttccgag

SEQ ID NO: 4 - DNA, 21 bp, Artificial sequence
  Synthetic construct
  misc_feature: Gene BAX
  misc_feature: Reverse primer sequence
ccagcccatgatggttctgat

SEQ ID NO: 5 - DNA, 20 bp, Artificial sequence
  Synthetic construct
  misc_feature: Gene BAK1
  misc_feature: Forward primer sequence
gagagcctgccctgccctct

SEQ ID NO: 6 - DNA, 20 bp, Artificial sequence
  Synthetic construct
  misc_feature: Gene BAK1
  misc_feature: Reverse primer sequence
ccacccagccacccctctgt

SEQ ID NO: 7 - DNA, 20 bp, Artificial sequence
  Synthetic construct
  misc_feature: Gene BIM
  misc_feature: Forward primer sequence
ggcaaagcaaccttctgatg

SEQ ID NO: 8 - DNA, 20 bp, Artificial sequence
  Synthetic construct
  misc_feature: Gene BIM
  misc_feature: Reverse primer sequence
taaccattcgtgggtggtct

SEQ ID NO: 9 - DNA, 21 bp, Artificial sequence
  Synthetic construct
  misc_feature: Gene PUMA
  misc_feature: Forward primer sequence
gacctcaacgcacagtacgag

SEQ ID NO: 10 - DNA, 22 bp, Artificial sequence
  Synthetic construct
  misc_feature: Gene PUMA
  misc_feature: Reverse primer sequence
aggagtcccatgatgagattgt

SEQ ID NO: 11 - DNA, 21 bp, Artificial sequence
  Synthetic construct
  misc_feature: Gene ATP6AP1
  misc_feature: Forward primer sequence
cagcgacttgcagctctctac

SEQ ID NO: 12 - DNA, 23 bp, Artificial sequence
  Synthetic construct
  misc_feature: Gene ATP6AP1
  misc_feature: Reverse primer sequence
tgaaatcctcaatgctcagcttg

SEQ ID NO: 13 - DNA, 22 bp, Artificial sequence
  Synthetic construct
  misc_feature: Gene ATP6V0D1
  misc_feature: Forward primer sequence
ttcccggagctttactttaacg

SEQ ID NO: 14 - DNA, 20 bp, Artificial sequence
  Synthetic construct
  misc_feature: Gene ATP6V0D1
  misc_feature: Reverse primer sequence
caagtcctctagcgtctcgc

SEQ ID NO: 15 - DNA, 22 bp, Artificial sequence
  Synthetic construct
  misc_feature: Gene ATP6V1E1
  misc_feature: Forward primer sequence
aacatagagaaaggtcggcttg

SEQ ID NO: 16 - DNA, 23 bp, Artificial sequence
  Synthetic construct
  misc_feature: Gene ATP6V1E1
  misc_feature: Reverse primer sequence
gactttgagtctcgcttgattca

SEQ ID NO: 17 - DNA, 21 bp, Artificial sequence
  Synthetic construct
  misc_feature: Gene ATP6V0E1
  misc_feature: Forward primer sequence
gtcctaaccggggagttatca

SEQ ID NO: 18 - DNA, 21 bp, Artificial sequence
  Synthetic construct
  misc_feature: Gene ATP6V0E1
  misc_feature: Reverse primer sequence
aaagagagggttgagttgggc

SEQ ID NO: 19 - DNA, 23 bp, Artificial sequence
  Synthetic construct
  misc_feature: Gene LAMP2
  misc_feature: Forward primer sequence
gaaaatgccacttgcctttatgc

SEQ ID NO: 20 - DNA, 20 bp, Artificial sequence
  Synthetic construct
  misc_feature: Gene LAMP2
  misc_feature: Reverse primer sequence
aggaaaagccaggtccgaac

SEQ ID NO: 21 - DNA, 20 bp, Artificial sequence
  Synthetic construct
  misc_feature: Gene LAMP3/CD63
  misc_feature: Forward primer sequence
cagtggtcatcatcgcagtg

SEQ ID NO: 22 - DNA, 21 bp, Artificial sequence
  Synthetic construct
  misc_feature: Gene LAMP3/CD63
  misc_feature: Reverse primer sequence
atcgaagcagtgtggttgttt

SEQ ID NO: 23 - DNA, 21 bp, Artificial sequence
  Synthetic construct
  misc_feature: Gene SQSTM1/p62
  misc_feature: Forward primer sequence
gactacgacttgtgtagcgtc

SEQ ID NO: 24 - DNA, 21 bp, Artificial sequence
  Synthetic construct
  misc_feature: Gene SQSTM1/p62
  misc_feature: Reverse primer sequence
agtgtccgtgtttcaccttcc

SEQ ID NO: 25 - DNA, 20 bp, Artificial sequence
  Synthetic construct
  misc_feature: Gene ATG2A
  misc_feature: Forward primer sequence
tgtccctgtagccatgttcg

SEQ ID NO: 26 - DNA, 21 bp, Artificial sequence
  Synthetic construct
  misc_feature: Gene ATG2A
  misc_feature: Reverse primer sequence
tcaggatctccgtgtactcag

SEQ ID NO: 27 - DNA, 22 bp, Artificial sequence
  Synthetic construct
  misc_feature: Gene CLCN5
  misc_feature: Forward primer sequence
ataggcaccgagagattaccaa

SEQ ID NO: 28 - DNA, 23 bp, Artificial sequence
  Synthetic construct
  misc_feature: Gene CLCN5
  misc_feature: Reverse primer sequence
ctaacgaacctgataaaagccca

SEQ ID NO: 29 - DNA, 22 bp, Artificial sequence
  Synthetic construct
  misc_feature: Gene CLCN6
  misc_feature: Forward primer sequence
tctccttacggaagatccagtt

SEQ ID NO: 30 - DNA, 21 bp, Artificial sequence
  Synthetic construct
  misc_feature: Gene CLCN6
  misc_feature: Reverse primer sequence
aaggtggcagacatggaacaa

SEQ ID NO: 31 - DNA, 22 bp, Artificial sequence
  Synthetic construct
  misc_feature: Gene CLCN7
  misc_feature: Forward primer sequence
ccacgttcaccctgaattttgt

SEQ ID NO: 32 - DNA, 22 bp, Artificial sequence
  Synthetic construct
  misc_feature: Gene CLCN7
  misc_feature: Reverse primer sequence
aaaccttccgaagttgatgagg

SEQ ID NO: 33 - DNA, 21 bp, Artificial sequence
  Synthetic construct
  misc_feature: Gene GAPDH
  misc_feature: Forward primer sequence
tgtgggcatcaatggatttgg

SEQ ID NO: 34 - DNA, 22 bp, Artificial sequence
  Synthetic construct
  misc_feature: Gene GAPDH
  misc_feature: Reverse primer sequence
acaccatgtattccgggtcaat

SEQ ID NO: 35 - protein, 10 aa, Artificial sequence
  Synthetic construct
  MISC_FEATURE (6)..(6): Xaa is the D-Ser(t-Bu)
  MISC_FEATURE (10)..(10): Xaa is Azgly
EHWSYXLRPX

(19) United States
(12) Patent Application Publication
(10) Pub. No.: US 2023/0348419 A1
(43) Pub. Date: Nov. 2, 2023

(54) BIAMINOQUINOLINES AND NANOFORMULATIONS FOR CANCER TREATMENT

(71) Applicant: The Regents of the University of California, Oakland, CA (US)

(72) Inventors: Yuanpei LI, Oakland, CA (US); Zhao MA, Oakland, CA (US)

(21) Appl. No.: 17/761,162

(22) PCT Filed: Sep. 18, 2020
(86) PCT No.: PCT/US2020/051430

§ 371 Date: Mar. 16, 2022

Related U.S. Application Data
(60) Provisional application No. 62/902,156, filed on Sep. 18, 2019.

(51) Int. Cl. C07D 401/12; A61K 45/06; A61K 51/12; C07J 43/00; C07D 401/14; C07D 487/22; B82Y 30/00; B82Y 5/00

(52) U.S. Cl. CPC C07D 401/12; A61K 45/06; A61K 51/1251; C07J 43/003; C07D 401/14; C07D 487/22; B82Y 30/00; B82Y 5/00

(57) ABSTRACT

The present invention provides bisaminoquinoline compounds of Formula (I). The present invention also provides nanocarriers comprising compounds of the present invention, and methods of using the nanocarriers for treating diseases and imaging.

### CROSS-REFERENCES TO RELATED APPLICATIONS

This application is a U.S. National Phase Application of PCT International Application No. PCT/US2020/051430, filed Sep. 18, 2020, which claims priority to U.S. Provisional Application No. 62/902,156 filed Sep. 18, 2019, which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. R01CA199668 and 5R01CA232845, awarded by the National Institutes of Health and National Cancer Institute, R01DE029237, awarded by National Institutes of Health and National Institute of Dental and Craniofacial Research, and R01HD086195, awarded by National Institutes of Health and National Institute of Child Health and Human Development. The Government has certain rights in the invention.

### INCORPORATION BY REFERENCE OF SEQUENCE LISTING

The contents of the text file named “052564-556N01US_Sequence_Listing_ST25.TXT”, which was created on May 18, 2022 and is 15 KB in size, are hereby incorporated by reference in their entirety.

### BACKGROUND OF THE INVENTION

The growing exploration of nanomedicine has contributed greatly to cancer treatment over the past few decades. Both carrier-assisted nanomedicines and carrier-free nanomedicines are being developed to improve drug-intrinsic kinetics and safety profiles. However, there are several limitations associated with these nanotherapeutic approaches. First, complexity and toxicity due to their multicomponent natures have severely hampered the clinical translation of many nanoformulations. Second, most drugs used in conventional delivery studies were approved decades ago and some are no longer first-line treatments. Third, nanoformulations that are designed for recently developed therapeutic agents, especially new chemical entities, may encounter patent issues. Finally, not all drugs can be structurally modified. These limitations can be addressed in the context of medicinal chemistry. Compared with nanomedicine, which focuses on delivery profiles for drug research and development, medicinal chemistry commits to the discovery of drug entities in earlier stages. Although drug discovery technologies have generated numerous drug leads and candidates, problems surrounding drug kinetics, metabolism and toxicology remain challenging. These challenges may also be solved relatively easily by nanotechnologies from the field of nanomedicine. To take advantage of this transdisciplinary connection, the principle of nanotechnology into initial drug design was integrated and developed a one-component non-prodrug nanomedicine (ONN) strategy (FIG. 1). In this strategy, the drug design follows both conventional drug design strategies and molecular self-assembly principles so that designed drugs are endowed with advantages from the perspectives of both drug discovery and drug delivery.

Lysosomes were chosen as therapeutic cancer targets. Cancer cell lysosomes are hypertrophic and easily ruptured and are more fragile than normal lysosomes. Lysosomal membrane permeabilization (LMP) can directly trigger cell death by enabling the release of proteolytic enzymes (i.e. cathepsins) into the cytoplasm; therefore, lysosomotropic detergents that can induce LMP have been developed for tumour treatment. Moreover, lysosomal inhibition has considerable potential as an anticancer strategy because it interferes with autophagy, an important pathway for the stress response and drug resistance of established tumours. The lysosomotropic alkalizers chloroquine (CQ) and hydroxychloroquine (HCQ) are commonly used autophagy inhibitors that have been tested in multiple clinical trials against various cancer types. However, their efficacy is considered insufficient, particularly when they are used as single agents.

Pharmacophore hybridization was adopted and molecular self-assembly to design a series of lipophilic cationic BAQ derivatives (FIG. 1 and FIG. 7). BAQ12 and BAQ13 are selected to construct ONNs because of their potential to be therapeutic agents and self-assembling building blocks. These BAQ ONNs display excellent anticancer activity in vitro, with enhanced effects on lysosomal disruption, lysosomal dysfunction and autophagy inhibition. Moreover, as nanodrugs, the BAQ ONNs exhibit the expected self-delivering profiles. These advantages from the perspectives of both drug discovery and drug delivery ultimately contribute to the significant anticancer activity of these compounds as single agents in gastrointestinal cancer models in vivo. In addition, the BAQ ONNs display promise for applications in combination therapy with napabucasin, as they play dual roles as both therapeutic agents and delivery carriers. With their multidisciplinary integration and ingenious functional superposition, BAQ ONNs will emerge as good alternatives for improvement of cancer treatment.

Integration of the unique advantages of the fields of drug discovery and drug delivery is invaluable for the advancement of drug development. Herein describes a self-delivering one-component new-chemical-entity nanomedicine (ONN) strategy to improve cancer therapy through incorporation of the self-assembly principle into drug design. A lysosomotropic detergent (MSDH) and an autophagy inhibitor (Lys05) are hybridized to develop bisaminoquinoline derivatives that can intrinsically form nanoassemblies. The selected BAQ12 and BAQ13 ONNs are highly effective in inducing lysosomal disruption, lysosomal dysfunction and autophagy blockade and exhibit 30-fold higher antiproliferative activity than hydroxychloroquine used in clinical trials. These single-drug nanoparticles demonstrate excellent pharmacokinetic and toxicological profiles and dramatic antitumour efficacy in vivo. In addition, they are able to encapsulate and deliver additional drugs to tumour sites and are thus promising agents for autophagy inhibition-based combination therapy. Given their transdisciplinary advantages, these BAQ ONNs have enormous potential to improve cancer therapy. What is needed are new BAQ ONNs. Surprisingly, the present invention meets this and other needs.

### BRIEF SUMMARY OF THE INVENTION

In one embodiment, the present invention provides a compound of formula (I):

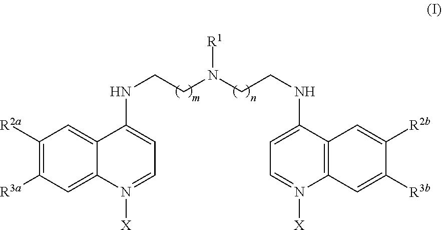

or a pharmaceutically acceptable salt thereof, wherein: R¹ is hydrogen, C₁₋₄₀ alkyl, C₂₋₄₀ alkenyl, C₂₋₄₀ alkynyl, —W, -(L-Y)ₚ—Z, or —C(O)R^{1a}, wherein each alkyl, alkenyl and alkynyl are optionally substituted with C₁₋₂₀ alkoxy, hydroxyl, or —NR^{1b}R^{1c}; W is C₃₋₁₂ cycloalkyl, C₆₋₁₂ aryl, or a 5 to 12 membered heteroaryl having 1 to 4 heteroatoms each independently N, O, or S, and wherein each cycloalkyl, aryl, and heteroaryl are optionally substituted with C₁₋₄₀ alkyl, C₂₋₄₀ alkenyl, or C₂₋₄₀ alkynyl; each L is independently absent, C₁₋₂₀ alkylene, C₂₋₂₀ alkenylene, or C₂₋₂₀ alkynylene; each Y is independently absent, —O—, —NH—, —NHC(O)—, —NHC(O)NH—, —NHSO₂—, —OC(O)—, —OC(O)NH—, —C(O)—, or —SO₂—; Z is a fluorophore, a photosensitizer, a porphyrin, a chemotherapeutic drug, a sterol, C₃₋₁₂ cycloalkyl, 3 to 12 membered heterocycloalkyl having 1 to 4 heteroatoms each independently N, O or S, C₆₋₁₂ aryl, 5 to 12 membered heteroaryl having 1 to 4 heteroatoms each independently N, O or S, —OH, or —NH₂; R^{1a} is C₁₋₄₀ alkyl, C₂₋₄₀ alkenyl, or C₂₋₄₀ alkynyl, wherein each alkyl, alkenyl and alkynyl are optionally substituted with C₁₋₄₀ alkoxy, hydroxyl, or —NR^{1b}R^{1c}; R^{1b} is hydrogen, C₁₋₄₀ alkyl, C₂₋₄₀ alkenyl, or C₂₋₄₀ alkynyl; R^{1c} is hydrogen, C₁₋₄₀ alkyl, C₂₋₄₀ alkenyl, C₂₋₄₀ alkynyl, or -L-W; R^{2a}, R^{2b}, R^{3a}, and R^{3b} are each independently hydrogen, C₁₋₄₀ alkyl, C₂₋₄₀ alkenyl, C₂₋₄₀ alkynyl, C₁₋₄₀ alkoxy, halogen, —CN, or —NO₂; R^{2a}, R^{2b}, R^{3a}, and R^{3b} are each independently hydrogen, C₁₋₄₀ alkyl, C₂₋₄₀ alkenyl, C₂₋₄₀ alkynyl, C₁₋₄₀ alkoxy, halogen, —CN, or —NO₂; m and n are independently an integer from 1 to 10; p is independently an integer from 1 to 20; and each X is independently absent or —O—, wherein when X is absent, R^{2a} and R^{2b} are hydrogen, and R^{3a} and R^{3b} are each independently hydrogen, -OMe, fluorine, chlorine, bromine, or —NO₂, then R¹ is C₂₋₄₀ alkyl, C₂40 alkenyl, C₄₋₄₀ alkynyl, —W, -(L-Y)ₚ—Z, or —C(O)R^{1a}, and wherein when X is absent, R¹ is —CH₂CH₂NH(7-chloro-4-quinolinyl), and R^{2a} and R^{2b} are hydrogen, then R^{3a} and R^{3b} are independently selected from hydrogen, C₁₋₂₀ alkyl, C₂₋₄₀ alkenyl, C₂₋₄₀ alkynyl, C₁₋₄₀ alkoxy, fluorine, bromine, iodine, —CN, or —NO₂.

In another embodiment, the present invention provides a nanocarrier having an interior and an exterior, the nanocarrier comprising a plurality of compounds of the present invention, or a pharmaceutically acceptable salt thereof, wherein each compound self-assembles in an aqueous solvent to form the nanocarrier such that a hydrophobic pocket is formed in the interior of the nanocarrier, and a hydrophilic group self-assembles on the exterior of the nanocarrier.

In another embodiment, the present invention provides a method of treating a disease, comprising administering to a subject in need thereof, a therapeutically effective amount of a nanocarrier of the present invention.

In another embodiment, the present invention provides a method of imaging, comprising administering to a subject to be imaged, an effective amount of a nanocarrier of the present invention.

### BRIEF DESCRIPTION OF THE DRAWINGS

FIG. 1 shows a schematic illustration of the proposed drug design strategy and the current work. Path (a) shows an interdisciplinary drug design strategy is proposed to integrate the conventional fields of medicinal chemistry and nanomedicine. Drugs are named as one-component non-prodrug nanomedicines (ONNs), which are designed according to the strategies of conventional drug design and molecular self-assembly so that they could acquire the advantages from the perspectives of both drug discovery and drug delivery. Path (b) shows the proof-of-concept experiment in this work: discovery of self-delivering lysosomotropic bisaminoquinoline (BAQ) derivatives for cancer therapy. The BAQ derivatives, generated from the hybridization of lysosomotropic detergents and the BAQ-based autophagy inhibitor, can self-assemble into BAQ ONNs that show enhanced functions in vitro, excellent delivery profiles and significant in vivo therapeutic effects as single agents. Moreover, they also possess high drug-loading efficiency to deliver the additional drug into tumour sites, thus generating a promising application of combination therapy.

FIGS. 2A-2G shows characterization of BAQ ONNs. FIG. 2A shows size change of BAQ NPs (10 μM) in acetate buffer with different pH values; Data are mean values=SD; n=3 independent nanoparticle samples. FIG. 2B shows the pH-dependent haemolysis induced by BAQ NPs (50 μM, 4 h) in PBS buffer; Data are mean values±SD; n=3 independent nanoparticle samples. FIG. 2C shows the pH change of BAQ NPs (1 mM) within hydrochloric acid (HCl, 0.1 M) titration. FIGS. 2D-2E show representative TEM micrograph at pH 7.4 (FIG. 2D) and pH 5.0 (FIG. 2E); The insets display the size distribution (left) and Tyndall effect (right); Experiments were all repeated three times independently. FIG. 2F show in vitro drug releasing patterns at pH 7.4 and pH 5.0; Data are mean values±SD; n=3 independent nanoparticle samples. FIG. 2G shows the count rate for various concentrations of BAQ NPs in water; The intersection of two lines refers to CACs of BAQ12 NPs (0.45 μg mL⁻¹, 0.76 μM) and BAQ13 NPs (0.15 μg mL⁻¹, 0.25 μM); Data are mean values±SD; n=3 independent nanoparticle samples.

FIGS. 3A-3K shows BAQ ONNs induced lysosomal disruption and inhibited autophagy in MIA PaCa-2 cells. FIG. 3A shows representative images for cellular uptake of nanoparticles; Dextran-AF488-loaded cells were incubated with DiD-labeled BAQ ONNs for 2 h; Experiments were repeated three times independently. FIG. 3B shows cells were treated as indicated (10 μM, 2 h) and were stained by LysoTracker Green; Experiments were repeated three times independently. FIG. 3C shows AO staining of cells within the indicated treatments (5 μM, 12 h); Experiments were repeated three times independently. FIG. 3D shows representative images of Dextran-AF488-loaded cells that were treated as indicated (5 μM, 12 h); Experiments were repeated three times independently. FIG. 3E shows Cathepsin B release from isolated lysosomes after treatments as indicated (25 μM, 12 h); Data are presented as mean values=SD; n=3. FIG. 3F shows western blotting. FIG. 3G shows normalized quantification analysis of gel blots in FIG. 3F; Data are presented as mean values±SD; n=3. FIG. 3H shows representative LC3B-GFP images for the indicated 4 h treatments; Experiments were repeated three times independently. FIG. 3I shows quantification of LC3B-GFP puncta per cell in h; Data are presented as mean values±SD; n=3. FIG. 3J shows representative TEM images of cells that were treated as indicated (2 μM, 48 h); Orange rectangle: region of interest; Purple arrows: autophagic vesicles; Red arrows: lysosomes. FIG. 3K shows the average diameter of lysosomes; Data are presented as mean values±SD; n=7. All statistical p values were calculated by one-way ANOVA with the Tukey's multiple comparison test. ns., not significant; *p<0.05; **p<0.01; ****p<0.0001.

FIGS. 4A-4J show BAQ ONNs altered the expression of lysosomal genes and caused cell death via apoptosis. FIG. 4A shows GSEA demonstrating the enrichment of lysosomal gene sets in MIA PaCa-2 cells treated with BAQ13 NPs (5 μM, 24 h). GSEA was performed with n=1,000 permutations, where p-adjust <0.05 and FDR <0.05 were considered significant. FIG. 4B shows representative upregulated lysosomal genes from a.

FIG. 4C shows comparison of gene upregulation between Lys05 and BAQ13 NPs. FIGS. 4D-4E show qPCR analysis of V-ATPase genes (FIG. 4D) and Cl-channel genes (FIG. 4E) involved in the indicated treatments (5 μM, 24 h); Data are mean values±SD; n=3. FIG. 4F shows viability curves of cells that were exposed to the 48 h treatments and the corresponding IC50 values; Data are mean values±SD; n=3. FIG. 4G shows MIA PaCa-2 (1.5 μM) and HT29 (1.0 μM) cell growth curves within continuous treatments; Data are mean values±SD; n=3 independent experiments. FIG. 4H shows clonogenic assay of MIA PaCa-2 and HT29 cells; n=3. FIG. 4I shows caspase 3/7 activity in MIA PaCa-2 and HT29 cells that were treated for 6 h and 12 h, respectively; Data are mean valuesf SD; n=4. FIG. 4J shows percentage of apoptotic population of MIA PaCa-2 (left) and HT29 (right) cells that were treated for 24 h. All the statistical p values were calculated by one-way ANOVA with the Tukey's multiple comparison test; ns., not significant; *p<0.05; **p<0.01; ***p<0.001; ****p<0.0001.

FIG. 5A-5H show the pharmacokinetics, biodistribution and in vivo antitumour effect of BAQ ONNs. FIG. 5A shows the plasma concentration-time profiles of DiD-loaded BAQ ONNs and free DiD after intravenous injection; Data are mean values=SD; n=3. FIG. 5B shows in vivo and ex vivo biodistribution of BAQ13 NPs in mice bearing HT29 tumour at 24 h post-injection. FIG. 5C shows quantitative fluorescence intensity of tissues obtained at 12 h and 24 h post-injection; Data are mean values±SD; n=3. FIG. 5D shows MIA PaCa-2 tumour growth curves in mice that were treated as indicated every three days; Data are mean values±SD; n=6 tumours per group. FIG. 5E shows body weight of mice during the treatment; Data are mean values±SD; n=6 mice per group. FIG. 5F shows weight of harvested tumours at the end of the treatment; Data are mean values±SD; n=6 tumours per group. (g-j) FIGS. 5G-J show representative H&E (FIG. 5G), IHC (FIG. 5H), immunoblotting (FIG. 5I) and TEM (FIG. 5J) results of tumours that were harvested at the end of treatments; Blots in i each group were from three individual tumours of each group; Purple arrows in FIG. 5J: autophagic vesicles; Experiments in FIGS. 5G-5J were all repeated three times independently. All statistical p values were calculated by one-way ANOVA with the Tukey's multiple comparison test; *p<0.05; ****p<0.0001.

FIGS. 6A-6L show BAQ ONNs have dual roles in the combination treatment. FIG. 6A shows establishment of the patient-derived pancreatic cancer stem cell (PCSC) model. FIG. 6B shows histological analysis showing the high-level stroma of PCSC tumours; Experiments were repeated three times independently. FIG. 6C shows viability curves of PCSCs that were treated for 48 h and the IC50 values; n=3 independent experiments. FIG. 6D shows AO staining to show the LMP of PCSC that were treated for 12 h; Experiments were repeated three times independently. FIG. 6E shows immunoblotting analysis of autophagy proteins in PCSC that were treated as indicated (2.5 μM, 48 h); Experiments were repeated three times independently. FIG. 6F shows synergistic effect of BAQ13 NPs and napabucasin (48 h). FIG. 3G shows tumour growth curves in subcutaneous MIA PaCa-2 xenograft model within intravenous administration every three days; Data are mean values±SD; n=10 tumours per group. FIG. 3H shows images of tumours that harvested at end of treatment. FIG. 3I shows mice body weight changes during treatment; Data are mean values±SD; n=5 mice per group. FIG. 3J shows representative images of PCSC tumour sections; Experiments were repeated three times independently. FIG. 3K shows in vivo and ex vivo fluorescence imaging of BAQ13 NPs co-loading with napabucasin and DiD in the PCSC model at 48 h post intravenous injection (10 mg kg⁻¹). FIG. 3L shows quantitative fluorescence intensity of tissues in FIG. 3K, Data are mean values±SD; n=3 mice per group. All statistical p values were calculated by the two-tailed Student's t-test. ns., not significant; *p<0.05; **p<0.01; ****p<0.0001.

FIG. 7 shows the chemical structures of compounds involved in this work and the synthetic route of BAQ12-BAQ18.

FIGS. 8A-8C show the in vitro evaluation of BAQ NPs. FIG. 8A shows size distribution. FIG. 8B shows observation of pH-dependent haemolytic effect; Red blood cells were treated as indicated (50 μM, 4 h). FIG. 8C shows viability curves of various cell lines that were exposed to different compounds for 24 h, respectively. Data are presented as mean values±SD; n=3 independent experiments.

FIGS. 9A-9G show the stability measurements and TEM characterization of BAQ NPs. FIGS. 9A-9B show Dynamic Light Scattering (DLS) measurements of BAQ12 NPs and BAQ13 NPs in neutral condition; Data are presented as mean values±SD; n=3. FIG. 9C shows whole appearance of BAQ12 NPs and BAQ13 NPs at Day 10 and 30. FIGS. 9D-9E show DLS measurements of BAQ12 NPs and BAQ13 NPs in presence of 10% FBS; Data are presented as mean values±SD; n=3. FIG. 9F shows size distribution of BAQ12 NPs (left) and BAQ13 NPs (right) at 24 h post-incubation with 0.5 mM BSA. FIG. 9G shows representative TEM images of BAQ13 NPs that loads different agents; Experiments were repeated three times independently.

FIG. 10A-10F show the effect of BAQ NPs on lysosomes and autophagy on cell level. FIG. 10A shows Pearson correlation coefficients for colocalization analysis of FIG. 3A. FIG. 10B shows fluorescence quantification of LysoTracker Green in MIA PaCa-2 cells that were treated as indicated; Data are presented as mean values±SD; n=3 independent experiments; Statistical significance was calculated by the two-tailed Student's t-test; **p<0.01. FIG. 10C shows HT29 cells were treated as indicated (10 μM, 2 h) and then were stained with LysoTracker Red; Experiments were repeated three times independently. FIG. 10D shows AO staining of HT29 cells treated as indicated (5 μM, 12 h); Experiments were repeated three times independently. FIG. 10E shows ratio of fluorescence intensity at 525 nm and 650 nm in MIA PaCa-2 cells that were treated as indicated for 12 h; Data are presented as mean values±SD; n=3 independent experiments; Statistical significance was calculated by one-way ANOVA with the Tukey's multiple comparison test; ns., not significant; ****p<0.0001. FIG. 10F shows representative LC3B-GFP images in cells within the corresponding treatments (5 μM, 4 h); Experiments were repeated three times independently.

FIG. 11A-11F show the effect of BAQ NPs on gene expression and lipid metabolism. FIG. 11A shows volcano plots from RNA-seq showing differentially expressed genes in MIA PaCa-2 cells induced by Lys05 (bottom) and BAQ13 NPs (upper). FIG. 11B shows change of autophagy-associated genes according to the RNA-seq results. FIG. 11C shows qPCR analysis of representative autophagy- and apoptosis-associated genes of MIA PaCa-2 cells that were treated as indicated (5 μM, 24 h); Data are presented as mean values±SD; n=3; Statistical significance was calculated by one-way ANOVA with the Tukey's multiple comparison test; *p<0.05; **p<0.01; ***p<0.001; ****p<0.0001. FIG. 11D shows change of apoptosis-associated genes of MIA PaCa-2 cells that were treated as indicated (5 μM, 12 h); Data are presented as mean values±SD; n=3; Statistical significance was calculated by the two-tailed Student's t-test; *p<0.05; **p<0.01. FIGS. 11E-11F show concentration alteration of acid sphingomyelinase (ASM) substrates (FIG. 11E) and phospholipase A (PLA) substrates (FIG. 11F) in MIA PaCa-2 cells with or without treatment (2.5 μM, 48 h); Data are presented as mean values±SD; n=4.

FIG. 12 shows the viability curves of various cell lines that were treated as indicated for 48 h. Data are presented as mean values±SD; n=3 independent experiments.

FIGS. 13A-13F show the biodistribution and toxicity studies of BAQ NPs. FIG. 13A shows in vivo and ex vivo imaging of mice bearing HT29 tumours after 12 h post-injection (i.v.) with free DiD (upper) and DiD-loaded BAQ13 NPs (lower), n=3 mice per group. FIG. 13B shows quantitative fluorescence intensity of organs in a; Data are presented as mean values±SD; n=3 mice per group; Data are presented as mean values±SD; n=3; Statistical significance was calculated by the two-tailed Student's t-test; ***p<0.001; ****p<0.0001. FIG. 13C shows concentration-dependent haemolysis induced by the corresponding treatments in physiological pH; Data are presented as mean values±SD; n=3 independent nanoparticle samples; Statistical significance was calculated by one-way ANOVA with the Tukey's multiple comparison test; ns., not significant; ***p<0.001; ****p<0.0001. FIG. 13D shows survival of FVB/n mice that were i.v. injected with the corresponding agents every two days; n=6 mice per group. FIG. 13E shows body weight of mice that were treated every two days as indicated; Data are presented as mean values±SD; n=4 mice per group. FIG. 13F show Dynamic Light Scattering (DLS) measurement and representative TEM image of liposomes@Lys05; Experiments were repeated three times independently.

FIGS. 14A-14E show Analysis of tissue sections and haematology. FIG. 14A shows H&E analysis of major organs from mice that were treated with vehicle (Saline), Lys05 (20 mg kg⁻¹, ip), BAQ12 NP (20 mg kg⁻¹, iv), and BAQ13 NP (20 mg kg⁻¹, iv) for 24 days. The scale bar is 100 μm. Experiments were repeated three times independently. FIG. 14B shows the corresponding serum chemistry analysis of mice in a. 1. Alanine Transaminase U L⁻¹, 2. Aspartate Transaminase U L⁻¹, 3. Blood Urea Nitrogen mg dL⁻¹, 4, Creatinine mg dL⁻¹, 5. Total Bilirubin mg dL⁻¹, 6. Hemolysis. FIGS. 14B-14E shows the corresponding complete blood count (CBC) analysis of mice in a; Data are presented as mean values±SD; n=3 mice per group. 1. Absolute Neutrophil cells (k L⁻¹), 2. Absolute Monocyte cells (k μL⁻¹), 3. Absolute Eosinophil cells (k μL⁻¹), 4. Absolute Basophil cells (k μL⁻¹), 5. Eosinophil %, 6. Basophil %, 7. MPV (fL), 8. Absolute Lymphocyte cells (k μL⁻¹), 9. WBC (k L⁻¹), 10. RBC (M μL⁻¹), 11. Hemoglobin (g dL⁻¹), 12. MCH (pg), 13. Monocyte (%), 14. RDW (%), 15. Neutrophil %, 16. Lymphocyte %, 17. Hematocrit %, 18. MCV (fL), 19. MCHC (g dL⁻¹), 20. Platelets (k μL⁻¹), 21. Presence of clots.

FIGS. 15A-15C show the in vivo evaluation of BAQ NPs in HT29 mouse model. FIG. 15A shows tumour growth curves in the subcutaneous HT29 xenograft model within intravenous administration every two days; Data are presented as mean values±SD; n=12 tumours per group. The statistical significance was calculated by the two-tailed Student's t-test; ns., not significant; *p<0.05; **p<0.01; ***p<0.001, ****p<0.0001. FIG. 15B shows body weight of mice during the treatment; Data are presented as mean values±SD; n=6 mice per group. FIG. 15C shows survival curves of mice; n=6 mice per group. The treatment was terminated on the 24th day.

FIG. 16 shows the gating strategy for isolation of pancreatic cancer stem cells (PCSCs).

FIGS. 17A-17F show the evaluation of BAQ NPs in the model of pancreatic cancer stem cells (PCSCs). FIG. 17A shows lysosomal deacidification assay. PCSCs were treated for 2 h (10 μM) and then were stained by LysoTracker Red; Experiments were repeated three times independently. FIG. 17B shows representative images of LC3B-GFP-expressing PCSCs that were treated as indicated (5 M, 4 h); Experiments were repeated three times independently. FIG. 17C shows percentage of the apoptotic population of PCSCs within the indicated treatments (5 μM, 24 h). FIG. 17D shows immunoblotting assay of PCSCs that were treated as indicated for 8 h; Experiments were repeated three times independently. FIG. 17E shows H&E analysis of major organs that were harvested at the end of treatments; Experiments were repeated three times independently. FIG. 17F shows biodistribution of free DiD and BAQ13 NPs@napabucasin+DiD in mice after 24 h post-injection by tail vein; Red circles: tumours.

FIGS. 18A-18G show Characterization of PBC NPs in physiological and lysosomal pH. FIG. 18A shows chemical structure of PBC monomer. FIG. 18B shows schematic illustration of transformability under various pH. FIG. 18C shows ultrafiltration analysis and TEM observation of PBC NPs at pH 7.4 and pH 5.0. FIG. 18D shows pH-dependent DLS measurements of PBC nanoparticles. FIG. 18E time-course DLS measurements of PBC nanoparticles at pH 5.0. FIG. 18F shows pH-dependent TEM measurements of PBC nanoparticles. FIG. 18G shows time-dependent TEM measurements of PBC nanoparticles.

FIGS. 19A-19H show Transformability of PBC nanoparticles induces lysosomal dysfunction and causes apoptosis in OSC-3 cells. FIG. 19A shows chemical structure of transformable PBC nanoparticles and non-transformable BAQ nanoparticles. FIG. 19B shows cell viability, FIG. 19C shows autophagy analysis, FIG. 19D shows apoptosis analysis, and FIG. 19E shows TEM images demonstrating the formed PBC nanofiber in lysosomes. FIG. 19F shows Acridine orange (AO) staining to showing lysosomal membrane (LMP) induced by PBC NPs. FIG. 19G shows dextran staining to show LMP induced by PBC NPs. (h) PBC NPs induced obvious vacuolation in OSC-3 cells.

FIGS. 20A-20L show PBC NPs showed a high photodynamic therapeutic efficacy and could overcome autophagy-associated drug resistance. FIGS. 20A-20B show the traditional photosensitizer pheophorbide a (Pa) induced autophagy in OSC-3 cells, which was verified by LC3-GFP-RFP imaging (FIG. 20A) and immunoblotting (FIG. 20B). FIG. 20C-20D shows Pa-mediated photodynamic therapy was sensitized by autophagy inhibitor Lys05, which was verified from cell viability (FIG. 20C) and immunoblotting (FIG. 20D). FIG. 20E shows the singlet oxygen production. FIG. 20F shows ROS production in OSC-3 cells. FIG. 20G show live/dead staining by DiO/PI of OSC-3 oral cells after different treatments. FIG. 20H shows cell viability. FIG. 20I shows apoptosis assay. FIG. 20J shows immunoblotting assay for apoptosis pathway. FIG. 20K shows TEM images of OSC-3 cells after different treatment. FIG. 20L shows immunoblotting assay for autophagy process.

FIG. 21 shows other synthesized BAQ derivatives and their cell viability results (48 h) in OSC-3 cells.

FIGS. 22A-22B show preliminary screening of BAQO derivatives. FIG. 22A shows chemical structures. FIG. 22B show viability result of pancreatic cancer stem cells (PCSCs) that were treated for 72 hr.

FIGS. 23A-23F show characterization of BAQ120 NPs. FIG. 23A shows DLS measurement of BAQ120 NPs. FIG. 23B shows representative TEM image of BAQ120 NPs. FIG. 23C shows measurement of critical aggregation concentration (CAC) of BAQ120 NPs. FIG. 23D-23E shows absorption (FIG. 23D) and fluorescence (FIG. 23E) spectra of BAQ120 NPs and free BAQ120 solution. FIG. 23F stability of BAQ120 NPs.

FIGS. 24A-24F show evaluation of antitumor activity of BAQ120 NPs in mice bearing PCSC tumors. FIG. 24A show tumor growth curve in mice that were treated (iv) every three days. FIG. 24B Weight of tumor that were collected at the end of treatment. FIG. 24C Body weight of mice during treatment. FIG. 24D Immunoblotting of autophagy process in mice. FIGS. 24E-24F show representative HE (FIG. 24E) and Ki67-IHC (FIG. 24F) images of tumors in different groups.

FIG. 25 shows chemical structures of designed BAQO derivatives.

### DETAILED DESCRIPTION OF THE INVENTION

### I. General

The present invention provides bisaminoquinoline derivative compounds, and nanocarriers formed from these compounds, which are useful for the treatment of diseases. The compounds and nanocarriers can target the lysosome, resulting in lysosomal disruption, lysosomal dysfunction, and/or autophagy inhibition. Further, the nanocarriers can be used in combination therapy by encapsulating additional drugs, or for co-administration with additional drugs, which can be useful for overcoming drug resistances. The nanocarriers can also be used for imaging cells or organisms of interest.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₁₋₂₀, C₁₋₃₀, C₁₋₄₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 40 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₂₋₂₀, C₂₋₃₀, C₂₋₄₀, C₃, C₃₋₄, C₃₋₅, C₃₋₆, C₄, C_{4- 5}, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene. Alkenylene groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₂₋₂₀, C₂₋₃₀, C₂₋₄₀, C₃, C₃₋₄, C₃₋₅, C₃₋₆, C₄, C_{4- 5}, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, isopropynylene, butynylene, sec-butynylene, pentynylene and hexynylene. Alkynylene groups can be substituted or unsubstituted.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbomane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Heterocycloalkyl” refers to a saturated ring system having from 3 to 12 ring members and from 1 to 4 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. The heteroatoms can also be oxidized, such as, but not limited to, —S(O)— and —S(O)₂—. Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Hydroxyl” refers to the —OH functional group.

“Halogen” refers to fluorine, chlorine, bromine and iodine.

“Fluorophore” refers to a chemical compound which emits lights, commonly in the 300-700 nm range, after excitation of the chemical compound. Upon absorption of transferred light energy (e.g., photon), a fluorophore goes into an excited state. As the molecule exits the excited state, it emits the light energy in the form of lower energy photon (e.g., emits fluorescence) and returns the dye molecule to its ground state. A fluorophore can be a natural chemical compound or a synthetic chemical compound. Fluorophores include, but are not limited to DAPI, ethidium bromide, acridine orange, GFP, mCherry, hydroxycoumarin, fluorescein, LysoTracker (red & green), Dextran-Alexa Fluor 488, Premo™ Autophagy Sensor LC3B-GFP, and Ac-DEVD-AMC.

“Photosensitizer” refers to compounds which can be activated by light in order to generate a reactive radical, typically a reactive oxygen species (ROS) for photodynamic therapy, but can also generate a reactive radical for polymerization, crosslinking, or degradation. Photosensitizers may be useful for treatment of diseases by producing singlet oxygen to damage tumours. Photosensitizers include, but are not limited to, porphyrins, dyes, and chlorophylls.

“Porphyrin” refers to any compound, with the following porphin core:

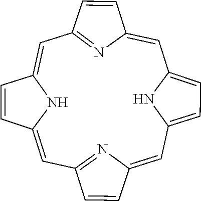 [SMILES: C1=Cc2cc3ccc(cc4nc(cc5ccc(cc1n2)[nH]5)C=C4)[nH]3]

wherein the porphin core can be substituted or unsubstituted.

“Sterol” refers to compounds with the following core structure:

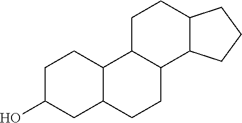 [SMILES: OC1CCC2C(CCC3C4CCCC4CCC23)C1]

wherein the core can be further substituted.

“Drug” refers to an agent capable of treating and/or ameliorating a condition or disease. A drug may be a hydrophobic drug, which is any drug that repels water.

Hydrophobic drugs useful in the present invention include, but are not limited to, hydrochloroquine (HCQ), Lys05, bortezomib, β-lapachone, JQ1, napabucasin, rapamycin, paclitaxel, SN38, etoposide, lenalidomide, and apoptozole. Other drugs includes non-steroidal anti-inflammatory drugs, and vinca alkaloids such as vinblastine and vincristine. The drugs of the present invention also include prodrug forms. One of skill in the art will appreciate that other drugs are useful in the present invention.

“Imaging agents” or “contrasting agents” refer to a compound which increases the contrast of structure within the location of the cell or body for imaging methods including, but not limited to MRI, PET, SPECT, and CT. Imaging agents can emit radiation, fluorescence, magnetic fields or radiowaves. Imaging agents include, but are not limited to radiometal chelators, radiometal atoms or ions, and fluorophores.

“Chemotherapeutic agent” refers to chemical drugs that can be used in the treatment of diseases such as, but not limited to, cancers, tumors and neoplasms. In some embodiments, a chemotherapeutic agent can be in the form of a prodrug which can be activated to a cytotoxic form. Chemotherapeutic agents commonly known by one of ordinary skill in the art can be used in the present invention. Chemotherapeutic agents include, but are not limited to daunorubicin, doxorubicin, paclitaxel, docetaxel, abraxane, bortezomib, etoposide, lenalidomide, apoptozole, carboplatin, cisplatin, oxaliplatin, vinblastine, and vincristine.

“Molecular targeted agent” refers to drugs which can target specific molecules involved in tumor and cancer evolution, growth, and spread. Targeting the specific molecules involved in tumor and cancer evolution can kill or inhibit tumor and cancer growth and spread. Molecular targeted agents include, but are not limited to trastuzumab, erlotinib, imatinib, nilotinib and vemurafenib.

“Immunotherapeutic agent” refers to a type of drug which can modify immune responses by stimulating or suppressing the immune system. Immunomodulatory agents include, but are not limited to HCQ, Lys05, JQT, rapamycin, napabucasin, ipilimumab, nivolumab, pembrolizumab, atezolizumab, avelumab, and durvalumab.

“Radiotherapeutic agent” refers to drugs which can be used in the treatment of diseases using radiotherapy. Radiotherapy is a disease treatment method which uses radiation to kill or inhibit tumor and cancer cells. Radiotherapeutic agents include, but are not limited to β-lapachone, cisplatin, nimorazole, cetuximab, misonidazole, and tirapazamine.

“Nanocarrier” or “nanoparticle” refers to a micelle resulting from aggregation of the compounds of the invention. The nanocarrier of the present invention can have a hydrophobic core and a hydrophilic exterior.

“Inhibition”, “inhibits” and “inhibitor” refer to a compound that prohibits or a method of prohibiting, a specific action or function.

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition; or, in some situations, preventing the onset of the symptom. The treatment or amelioration of symptoms can be based on any objective or subjective parameter; including, e.g., the result of a physical examination.

“Disease” refers abnormal cellular function in an organism, which is not due to a direct result of a physical or external injury. Diseases can refer to any condition that causes distress, dysfunction, disabilities, disorders, infections, pain, or even death. Diseases include, but are not limited to hereditary diseases such as genetic and non-genetic diseases, infectious diseases, non-infectious diseases such as cancer, deficiency diseases, and physiological diseases.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to animals such as mammals, including, but not limited to, primates (e.g., humans), cows, sheep, goats, horses, dogs, cats, rabbits, rats, mice and the like. In certain embodiments, the subject is a human.

“Therapeutically effective amount or dose” or “therapeutically sufficient amount or dose” or “effective or sufficient amount or dose” refer to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Target” or “targeting” refers to using a compound, protein, or antibody that specifically or preferentially binds to a cell, viral particle, viral protein, an antigen, or a biomolecule, or that is localized to a specific cell type, tissue type, microbe type, or viral type.

“Imaging” refers to using a device outside of the subject to determine the location of an imaging agent, such as a compound of the present invention. Examples of imaging tools include, but are not limited to, positron emission tomography (PET), magnetic resonance imaging (MRI), ultrasound, single photon emission computed tomography (SPECT) x-ray computed tomography (CT). The positron emission tomography detects radiation from the emission of positrons by an imaging agent.

### II. Compounds

In some embodiments, the present invention provides a compound of formula (I):

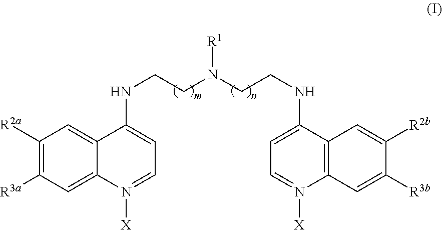

or a pharmaceutically acceptable salt thereof, wherein: R¹ is hydrogen, C₁₋₄₀ alkyl, C₂₋₄₀ alkenyl, C₂₋₄₀ alkynyl, —W, -(L-Y)ₚ—Z, or —C(O)R^{1a}, wherein each alkyl, alkenyl and alkynyl are optionally substituted with C₁₋₂₀ alkoxy, hydroxyl, or —NR^{1b}R^{1c}; W is C₃₋₁₂ cycloalkyl, C₆-12 aryl, or a 5 to 12 membered heteroaryl having 1 to 4 heteroatoms each independently N, O, or S, and wherein each cycloalkyl, aryl, and heteroaryl are optionally substituted with C₁₋₄₀ alkyl, C₂₋₄₀ alkenyl, or C₂₋₄₀ alkynyl; each L is independently absent, C₁₋₂₀ alkylene, C₂₋₂₀ alkenylene, or C₂₋₂₀ alkynylene; each Y is independently absent, —O—, —NH—, —NHC(O)—, —NHC(O)NH—, —NHSO₂—, —OC(O)—, —OC(O)NH—, —C(O)—, or —SO₂—; Z is a fluorophore, a photosensitizer, a porphyrin, a chemotherapeutic drug, a sterol, C₃₋₁₂ cycloalkyl, 3 to 12 membered heterocycloalkyl having 1 to 4 heteroatoms each independently N, O or S, C₆₋₁₂ aryl, 5 to 12 membered heteroaryl having 1 to 4 heteroatoms each independently N, O or S, —OH, or —NH₂; R^{1a} is C₁₋₄₀ alkyl, C₂₋₄₀ alkenyl, or C₂₋₄₀ alkynyl, wherein each alkyl, alkenyl and alkynyl are optionally substituted with C₁₋₄₀ alkoxy, hydroxyl, or —NR^{1b}R^{1c}; R^{1b} is hydrogen, C₁₋₄₀ alkyl, C₂₋₄₀ alkenyl, or C₂₋₄₀ alkynyl; R^{1c} is hydrogen, C₁₋₄₀ alkyl, C₂₋₄₀ alkenyl, C₂₋₄₀ alkynyl, or -L-W; R^{2a}, R^{2b}, R^{3a}, and R^{3b} are each independently hydrogen, C₁₋₄₀ alkyl, C₂₋₄₀ alkenyl, C₂₋₄₀ alkynyl, C₁₋₄₀ alkoxy, halogen, —CN, or —NO₂; m and n are independently an integer from 1 to 10; p is independently an integer from 1 to 20; and each X is independently absent or —O—, wherein when X is absent, R^{2a} and R^{2b} are hydrogen, and R^{3a} and R^{3b} are each independently hydrogen, -OMe, fluorine, chlorine, bromine, or —NO₂, then R¹ is C₂₋₄₀ alkyl, C₂₋₄₀ alkenyl, C₄₋₄₀ alkynyl, —W, -(L-Y)ₚ—Z, or —C(O)R^{1a}, and wherein when X is absent, R¹ is —CH₂CH₂NH(7-chloro-4-quinolinyl), and R^{2a} and R^{2b} are hydrogen, then R^{3a} and R^{3b} are independently selected from hydrogen, C₁₋₂₀ alkyl, C₂₋₄₀ alkenyl, C₂₋₄₀ alkynyl, C₁₋₄₀ alkoxy, fluorine, bromine, iodine, —CN, or —NO₂.

In some embodiments, the present invention provides a compound of Formula (I), wherein: R¹ is hydrogen, C₁₋₂₀ alkyl, C₂₋₂₀ alkenyl, C₂₋₂₀ alkynyl, —W, -(L-Y)ₚ—Z, or —C(O)R^{1a}, wherein each alkyl, alkenyl and alkynyl are optionally substituted with C₁₋₂₀ alkoxy, hydroxyl, or —NR^{1b}R^{1c}; W is C₃₋₁₂ cycloalkyl, C₆₋₁₂ aryl, or C₄₋₁₂ heteroaryl, wherein each cycloalkyl, aryl, and heteroaryl are optionally substituted with C₁₋₂₀ alkyl, C₂₋₂₀ alkenyl, or C₂-20 alkynyl; each L is independently absent, C₁₋₁₀ alkylene, C₂₋₁₀ alkenylene, or C₂₋₁₀ alkynylene; each Y is independently absent, —O—, —NH—, —NHC(O)—, —NHC(O)NH—, —NHSO₂—, —OC(O)—, —OC(O)NH—, —C(O)—, or —SO₂—; Z is a fluorophore, a photosensitizer, a porphyrin, a chemotherapeutic drug, or a sterol; R^{1a} is C₁₋₂₀ alkyl, C₂₋₂₀ alkenyl, or C₂₋₂₀ alkynyl, wherein each alkyl, alkenyl and alkynyl are optionally substituted with C₁₋₂₀ alkoxy, hydroxyl, or —NR^{1b}R^{1c}, R^{1b} is hydrogen, C₁₋₂₀ alkyl, C₂₋₂₀ alkenyl, or C₂₋₂₀ alkynyl, and R^{1c} is hydrogen, C₁₋₂₀ alkyl, C₂₋₂₀ alkenyl, C₂₋₂₀ alkynyl, or -L-W; R^{2a}, R^{2b}, R^{3a}, and R^{3b} are each independently hydrogen, C₁₋₂₀ alkyl, C₂₋₂₀ alkenyl, C₂₋₂₀ alkynyl, C₁₋₂₀ alkoxy, halogen, —CN, or —NO₂; m and n are independently an integer from 1 to 10; p is independently an integer from 1 to 20; each X is independently absent or —O⁻; wherein when X is absent, R^{2a} and R^{2b} are hydrogen, and R^{3a} and R^{3b} are each independently hydrogen, -OMe, fluorine, chlorine, bromine, or —NO₂, then R¹ is C₂₋₂₀ alkyl, C₂₋₂₀ alkenyl, C₄₋₂₀ alkynyl, —W, -(L-Y)ₚ—Z, or —C(O)R^{1a}; and wherein when X is absent, R¹ is —CH₂CH₂NH(7-chloro-4-quinolinyl), and R^{2a} and R^{2b} are hydrogen, then R^{3a} and R^{3b} are independently selected from hydrogen, C₁₋₂₀ alkyl, C₂₋₂₀ alkenyl, C₂₋₂₀ alkynyl, C₁₋₂₀ alkoxy, fluorine, bromine, iodine, —CN, or —NO₂.

In some embodiments, R¹ is hydrogen, C₁₋₂₀ alkyl, C₂₋₂₀ alkenyl, C₂₋₂₀ alkynyl, —W, -(L-Y)ₚ—Z, or —C(O)R^{1a}, wherein each alkyl, alkenyl and alkynyl are optionally substituted with C₁₋₂₀ alkoxy, hydroxyl, or —NR^{1b}R^{1c}; W is C₃₋₁₂ cycloalkyl, C₆₋₁₂ aryl, or 5 to 12 membered heteroaryl, wherein the 5 to 12 membered heteroaryl have 1 to 4 heteroatoms of N, O, and S, and wherein each cycloalkyl, aryl, and heteroaryl are optionally substituted with C₁₋₂₀ alkyl, C₂₋₂₀ alkenyl, or C₂₋₂₀ alkynyl; each L is independently absent, C₁₋₁₀ alkylene, C₂₋₁₀ alkenylene, or C₂₋₁₀ alkynylene; each Y is independently absent, —O—, —NH—, —NHC(O)—, —NHC(O)NH—, —NHSO₂—, —OC(O)—, —OC(O)NH—, —C(O)—, or —SO₂—; Z is a fluorophore, a photosensitizer, a porphyrin, a chemotherapeutic drug, or a sterol; R^{1a} is C₁₋₂₀ alkyl, C₂₋₂₀ alkenyl, or C₂₋₂₀ alkynyl, wherein each alkyl, alkenyl and alkynyl are optionally substituted with C₁₋₂₀ alkoxy, hydroxyl, or —NR^{1b}R^{1c}; R^{1b} is hydrogen, C₁₋₂₀ alkyl, C₂₋₂₀ alkenyl, or C₂₋₂₀ alkynyl; R^{1c} is hydrogen, C₁₋₂₀ alkyl, C₂₋₂₀ alkenyl, C₂₋₂₀ alkynyl, or -L-W; R^{2a}, R^{2b}, R^{3a}, and R^{3b} are each independently hydrogen, C₁₋₂₀ alkyl, C₂₋₂₀ alkenyl, C₂₋₂₀ alkynyl, C₁₋₂₀ alkoxy, halogen, —CN, or —NO₂; m and n are independently an integer from 1 to 10; p is independently an integer from 1 to 20; and each X is independently absent or —O—, wherein when X is absent, R^{2a} and R^{2b} are hydrogen, and R^{3a} and R^{3b} are each independently hydrogen, -OMe, fluorine, chlorine, bromine, or —NO₂, then R¹ is C₂₋₂₀ alkyl, C₂₋₂₀ alkenyl, C₄₋₂₀ alkynyl, —W, -(L-Y)ₚ—Z, or —C(O)R^{1a}, and wherein when X is absent, R¹ is —CH₂CH₂NH(7-chloro-4-quinolinyl), and R^{2a} and R^{2b} are hydrogen, then R^{3a} and R^{3b} are independently selected from hydrogen, C₁₋₂₀ alkyl, C₂₋₂₀ alkenyl, C₂₋₂₀ alkynyl, C₁₋₂₀ alkoxy, fluorine, bromine, iodine, —CN, or —NO₂.

In some embodiments, R¹ is hydrogen, C₁₋₄₀ alkyl, C₂₋₄₀ alkenyl, C₂₋₄₀ alkynyl, —W, -(L-Y)ₚ—Z, or —C(O)R^{1a}. In some embodiments, R¹ is C₁₋₄₀ alkyl, C₂₋₄₀ alkenyl, -(L-Y)ₚ—Z, or —C(O)R^{1a}.

In some embodiments, R¹ is C₁₋₄₀ alkyl. In some embodiments, R¹ is C₁₋₂₅ alkyl. In some embodiments, R¹ is C₁₋₂₀ alkyl. In some embodiments, R¹ is C₁₀₋₂₅ alkyl. In some embodiments, R¹ is C₁₀₋₂₀ alkyl. In some embodiments, R¹ is C₁₂₋₂₂ alkyl. In some embodiments, R¹ is C₁₂₋₁₈ alkyl.

In some embodiments, R¹ is C₂₋₄₀ alkenyl. In some embodiments, R¹ is C₂₀₋₄₀ alkenyl. In some embodiments, R¹ is C₃₀₋₄₀ alkenyl. In some embodiments, R¹ is C₂₋₄₀ alkynyl. In some embodiments, R¹ is C₂₀₋₄₀ alkynyl. In some embodiments, R¹ is C₃₀₋₄₀ alkynyl.

In some embodiments, R¹ is W. In some embodiments, W is C₃₋₁₂ cycloalkyl, C₆₋₁₂ aryl, or a 5 to 12 membered heteroaryl having 1 to 4 heteroatoms each independently N, O, or S, and wherein each cycloalkyl, aryl, and heteroaryl are optionally substituted with C₁₋₄₀ alkyl, C₂₋₄₀ alkenyl, or C₂₋₄₀ alkynyl. In some embodiments, W is C₅₋₁₂ cycloalkyl, C₆₋₁₂ aryl, or a 5 to 12 membered heteroaryl having 1 to 4 heteroatoms each independently N, O, or S. In some embodiments, W is C₅₋₁₂ cycloalkyl. In some embodiments, W is C₅₋₈ cycloalkyl. In some embodiments, W is cyclopentyl or cyclohexyl.

In some embodiments, R¹ is -(L-Y)ₚ—Z. In some embodiments, p is an integer from 1 to 20. In some embodiments, p is an integer from 1 to 10. In some embodiments, p is an integer from 1 to 5. In some embodiments, p is 1, 2, 3, 4, or 5. In some embodiments, p is 1.

In some embodiments, L is C₁₋₂₀ alkylene, C₂₋₂₀ alkenylene, or C₂₋₂₀ alkynylene. In some embodiments, L is C₁₋₂₀ alkylene. In some embodiments, L is C₁₋₁₀ alkylene. In some embodiments, L is C₁₋₅ alkylene.

In some embodiments, Y is absent, —O—, —NH—, —NHC(O)—, —NHC(O)NH—, —NHSO₂—, —OC(O)—, —OC(O)NH—, —C(O)—, or —SO₂—. In some embodiments, Y is absent, —NH—, —NHC(O)—, —NHC(O)NH—, —OC(O)—, —OC(O)NH—, or —C(O)—. In some embodiments, Y is absent, —NH—, —NHC(O)—, or —NHC(O)NH—. In some embodiments, Y is absent, —NH—, or —NHC(O)—.

In some embodiments, Z is a fluorophore, a photosensitizer, a porphyrin, a chemotherapeutic drug, a sterol, C₃₋₁₂ cycloalkyl, 3 to 12 membered heterocycloalkyl having 1 to 4 heteroatoms each independently N, O or S, C₆₋₁₂ aryl, 5 to 12 membered heteroaryl having 1 to 4 heteroatoms each independently N, O or S, —OH, or —NH₂.

Photosensitizers useful in the present invention include, but are not limited to, porphyrins, benzoporphyrins, corrins, chlorins, bacteriochlorophylls, corphins, or derivatives thereof. Representative photosensitizers are shown below:

| PHOTOSENSITIZERS | STRUCTURE |
|---|---|
| Porphyrin | 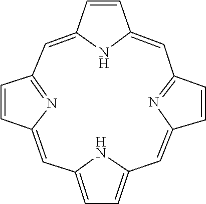 [SMILES: C1=Cc2cc3ccc(cc4nc(cc5ccc(cc1n2)[nH]5)C=C4)[nH]3] |
| Pyropheophorbide-a | 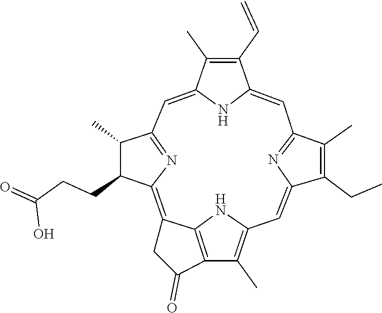 [SMILES: C=Cc1c(C)c2cc3nc(c4c5[nH]c(cc6nc(cc1[nH]2)C(C)=C6CC)c(C)c5C(=O)C4)[C@@H](CCC(=O)O)[C@@H]3C] |
| Pheophorbide | 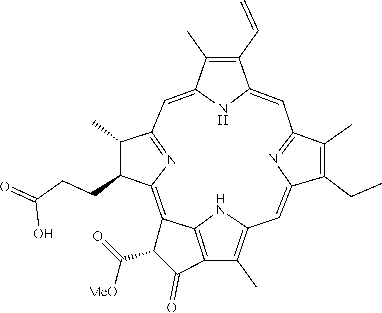 [SMILES: C=Cc1c(C)c2cc3nc(c4c5[nH]c(cc6nc(cc1[nH]2)C(C)=C6CC)c(C)c5C(=O)[C@@H]4C(=O)OC)[C@@H](CCC(=O)O)[C@@H]3C] |
| Chlorin e6 | 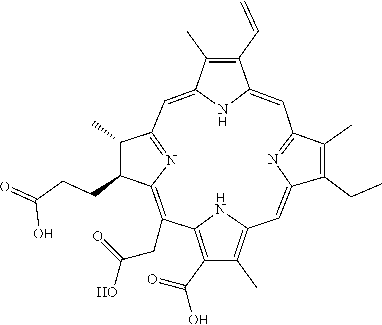 [SMILES: C=Cc1c(C)c2cc3nc(c(CC(=O)O)c4[nH]c(cc5nc(cc1[nH]2)C(C)=C5CC)c(C)c4C(=O)O)[C@@H](CCC(=O)O)[C@@H]3C] |
| Purpurin | 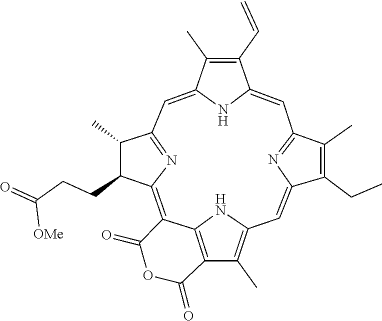 [SMILES: C=Cc1c(C)c2cc3nc(c4c5[nH]c(cc6nc(cc1[nH]2)C(C)=C6CC)c(C)c5C(=O)OC4=O)[C@@H](CCC(=O)OC)[C@@H]3C] |
| Purpurinimide | 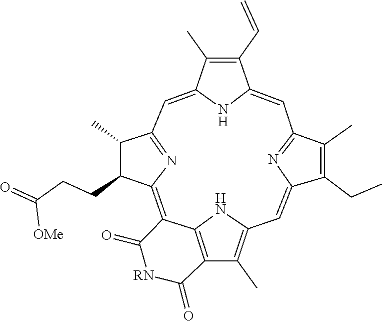 |
| Corrin | 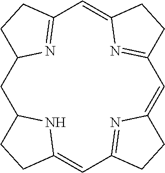 [SMILES: C1=C2/CCC(=N2)/C=C2/CCC(CC3CCC(=N3)/C=C3/CCC/1=N3)N2] |
| Chlorin | 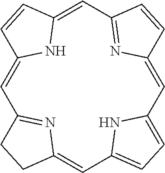 [SMILES: C1=Cc2cc3ccc(cc4nc(cc5ccc(cc1n2)[nH]5)CC4)[nH]3] |
| Corphin | 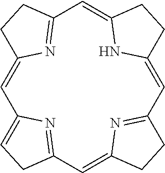 [SMILES: C1=C2/C=C3/CCC(=N3)/C=C3/CC/C(=C/C4=NC(=C\C(=N2)C1)/CC4)N3] |

In some embodiments, the photosensitizer is porphyrin, benzoporphyrin, corrin, chlorin, bacteriochlorophyll, corphin, or derivatives thereof. In some embodiments, the photosensitizer compound is porphyrin, pyropheophorbide-a, pheophorbide, chlorin e6, purpurin, purpurinimide, verteporfin, photofrin porfimer, rostaporfin, talporfin, or temoporfin. In some embodiments, the photosensitizer is pyropheophorbide-a. In some embodiments, the photosensitizer is pheophorbide-a. In some embodiments, the photosensitizer is porphyrin.

Any suitable porphyrin can be used in the compounds of the present invention. Representative porphyrins suitable in the present invention include, but are not limited to, pyropheophorbide-a, pheophorbide, chlorin e6, purpurin or purpurinimide. In some embodiments, the porphyrin can be pheophorbide-a. In some embodiments, the porphyrin can be pyropheophorbide-a.

In some embodiments, Z is a porphyrin, a sterol, 6 to 12 membered heterocycloalkyl, 8 to 12 membered heteroaryl, —OH, or —NH₂, wherein the 6 to 12 membered heterocycloalkyl and the 8 to 12 membered heteroaryl have 1 to 4 heteroatoms of N, O, and S. In some embodiments Z is porphyrin, cholic acid, indoline, isoindoline, 1-isoindolinone, pthalimide, phthalic anhydride, —OH, or —NH₂.

In some embodiments, p is 1; L is C₄₋₅ alkylene; Y is absent, —NH—, or —NHC(O)—; and Z is porphyrin, cholic acid, isoindoline, phthalimide, —OH, or —NH₂.

In some embodiments, R¹ is —C(O)R^{1a}. In some embodiments, R^{1a} is C₁₋₄₀ alkyl, C₂₋₄₀ alkenyl, or C₂₋₄₀ alkynyl, wherein each alkyl, alkenyl and alkynyl are optionally substituted with C₁₋₄₀ alkoxy, hydroxyl, or —NR^{1b}R^{1c}. In some embodiments, R^{1a} is C₁₋₂₀ alkyl, C₂₋₂₀ alkenyl, or C₂₋₂₀ alkynyl, wherein each alkyl, alkenyl and alkynyl are optionally substituted with C₁₋₂₀ alkoxy, hydroxyl, or —NR^{1b}R^{1c}. In some embodiments, R^{1a} is C₁₋₁₀ alkyl, C₂₋₁₀ alkenyl, or C₂₋₁₀ alkynyl. In some embodiments, R^{1a} is C₁₋₁₀ alkyl. In some embodiments, R^{1a} is C₁₋₅ alkyl. In some embodiments, R^{1a} is methyl, ethyl, propyl, or butyl.

In some embodiments, R^{1b} is C₁₋₄₀ alkyl, C₂₋₄₀ alkenyl, or C₂₋₄₀ alkynyl. In some embodiments, R^{1b} is C₁₋₂₀ alkyl, C₂₋₂₀ alkenyl, or C₂₋₂₀ alkynyl. In some embodiments, R^{1b} is C₁₋₁₀ alkyl. In some embodiments, R^{1b} is C₁₋₅ alkyl. In some embodiments, R^{1b} is methyl, ethyl, propyl, or butyl.

In some embodiments, Rio is C₁₋₄₀ alkyl, C₂₋₄₀ alkenyl, C₂₋₄₀ alkynyl, or -L-W. In some embodiments, R^{1c} is C₁₋₂₀ alkyl, C₂₋₂₀ alkenyl, C₂₋₂₀ alkynyl, or -L-W. In some embodiments, R^{1c} is C₁₋₁₀ alkyl. In some embodiments, R^{1c} is C₁₋₅ alkyl. In some embodiments, R^{1c} is methyl, ethyl, propyl, or butyl.

In some embodiments, R^{2a} and R^{2b} are each independently hydrogen, C₁₋₂₀ alkyl, C₂-alkenyl, C₂₋₂₀ alkynyl, C₁₋₂₀ alkoxy, halogen, —CN, or —NO₂. In some embodiments, R^{2a} and R^{2b} are each independently hydrogen, C₁₋₂₀ alkyl, or halogen. In some embodiments, R^{2a} and R^{2b} are each independently hydrogen or halogen. In some embodiments, R^{2a} and R^{2b} are each independently hydrogen, fluorine, chlorine, bromine, or iodine. In some embodiments, R^{2a} and R^{2b} are each independently hydrogen.

In some embodiments, R^{3a} and R^{3b} are each independently hydrogen, C₁₋₂₀ alkyl, C₂₋₂₀ alkenyl, C₂₋₂₀ alkynyl, C₁₋₂₀ alkoxy, halogen, —CN, or —NO₂. In some embodiments, R^{3a} and R^{3b} are each independently hydrogen, C₁₋₂₀ alkyl, or halogen. In some embodiments, R^{3a} and R^{3b} are each independently hydrogen or halogen. In some embodiments, R^{3a} and R^{3b} are each independently hydrogen, fluorine, chlorine, bromine, or iodine. In some embodiments, R^{3a} and R^{3b} are each independently chlorine.

In some embodiments, m and n are independently an integer from 1 to 10. In some embodiments, m and n are independently an integer from 1 to 5. In some embodiments, m and n are independently 1, 2, 3, 4, or 5. In some embodiments, m and n are each independently 1.

In some embodiments, each X is independently absent or —O⁻. In some embodiments, each X is absent. In some embodiments, each X is —O⁻.

In some embodiments, each X is absent. In some embodiments, the compound is the compound of Formula (Ia):

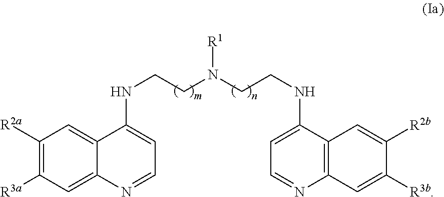

In some embodiments, R¹ is C₁₋₂₀ alkyl, and the compound is formula (Ia):

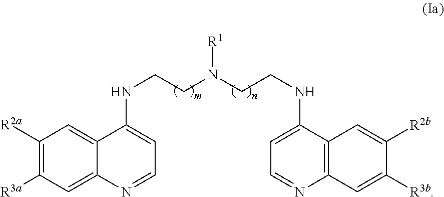

In some embodiments, the compound has the structure:

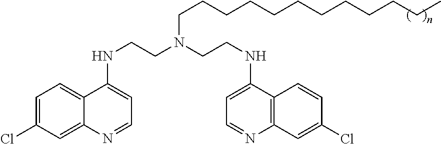

wherein n is an integer from 1 to 7.

In some embodiments, the compound is selected from the group consisting of:

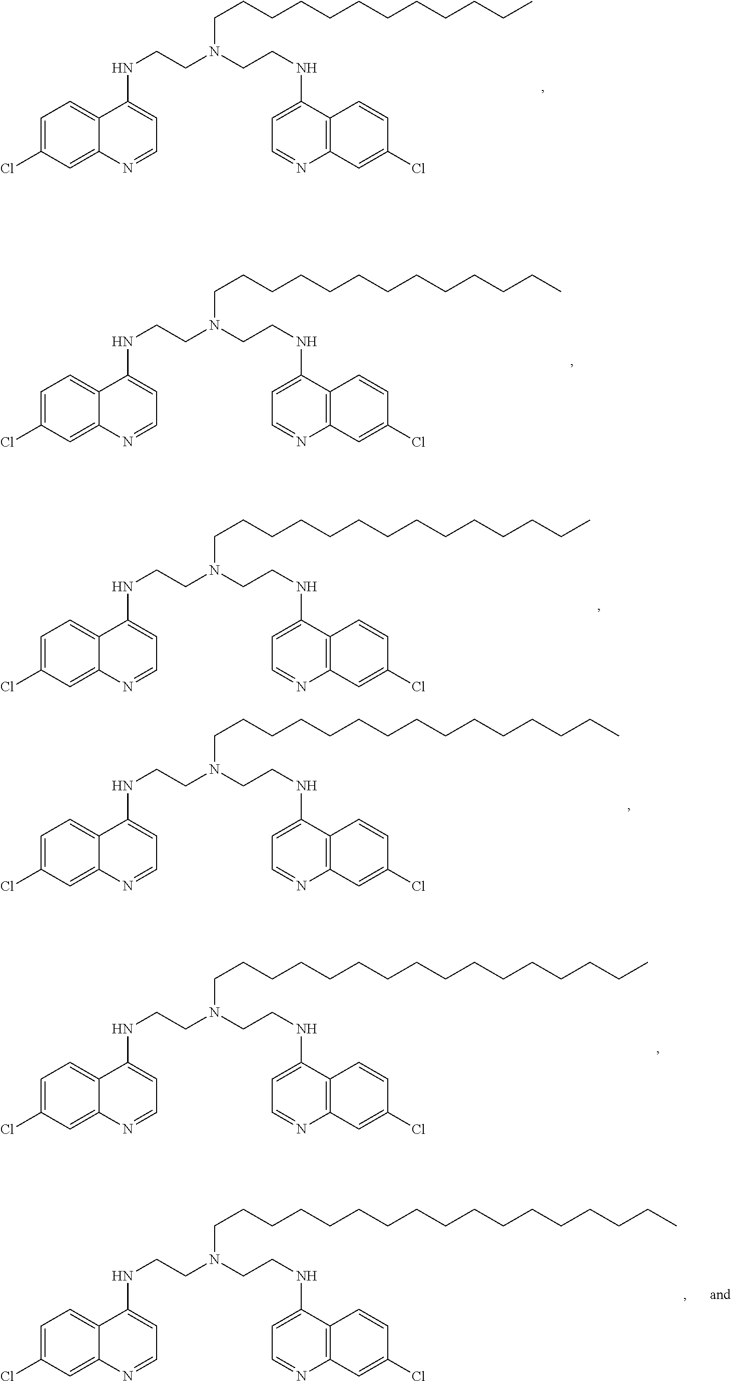

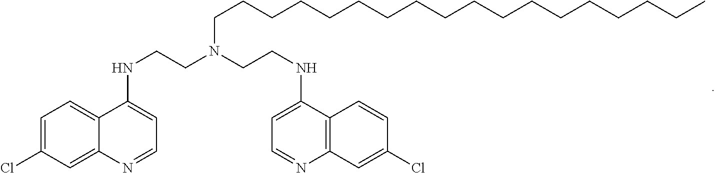 [SMILES: CCCCCCCCCCCCCCCCCCN(CCNc1ccnc2cc(Cl)ccc12)CCNc1ccnc2cc(Cl)ccc12]

In some embodiments, the compound is:

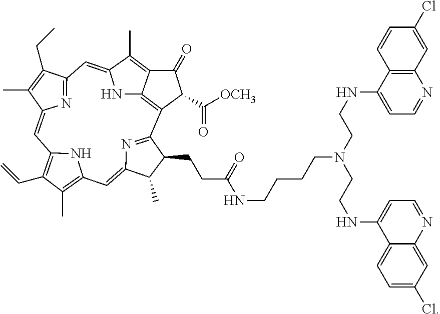 [SMILES: C=Cc1c(C)c2cc3nc(c4c5[nH]c(cc6nc(cc1[nH]2)C(C)=C6CC)c(C)c5C(=O)[C@@H]4C(=O)OC)[C@@H](CCC(=O)NCCCCN(CCNc1ccnc2cc(Cl)ccc12)CCNc1ccnc2cc(Cl)ccc12)[C@@H]3C]

In some embodiments, the compound is selected from the group consisting of:

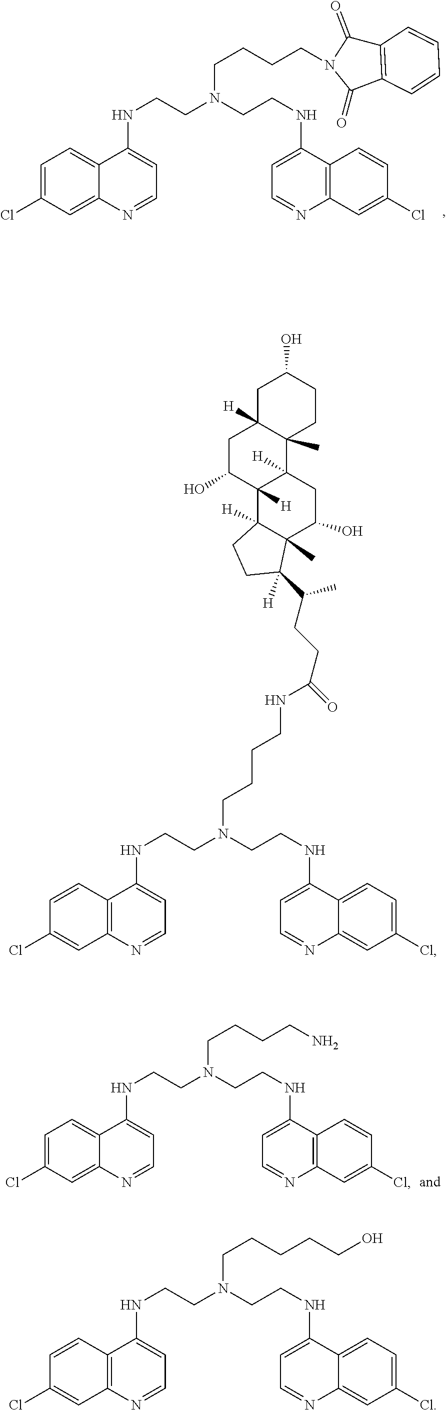

In some embodiments, each X is —O⁻. In some embodiments, the compound is the compound of Formula (Ib):

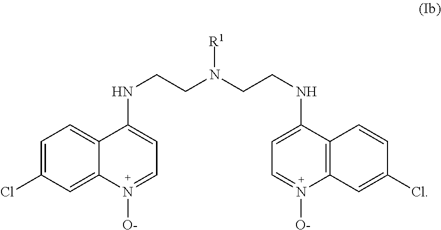

In some embodiments, the compound is selected form the group consisting of:

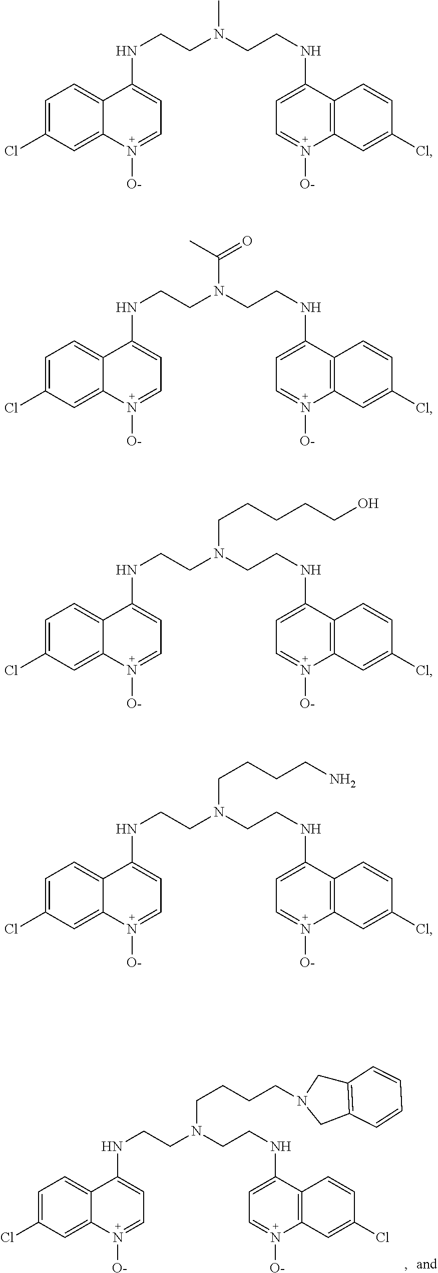

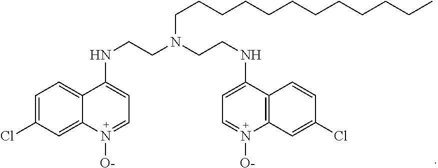

In some embodiments, the compound is selected from the group consisting of:

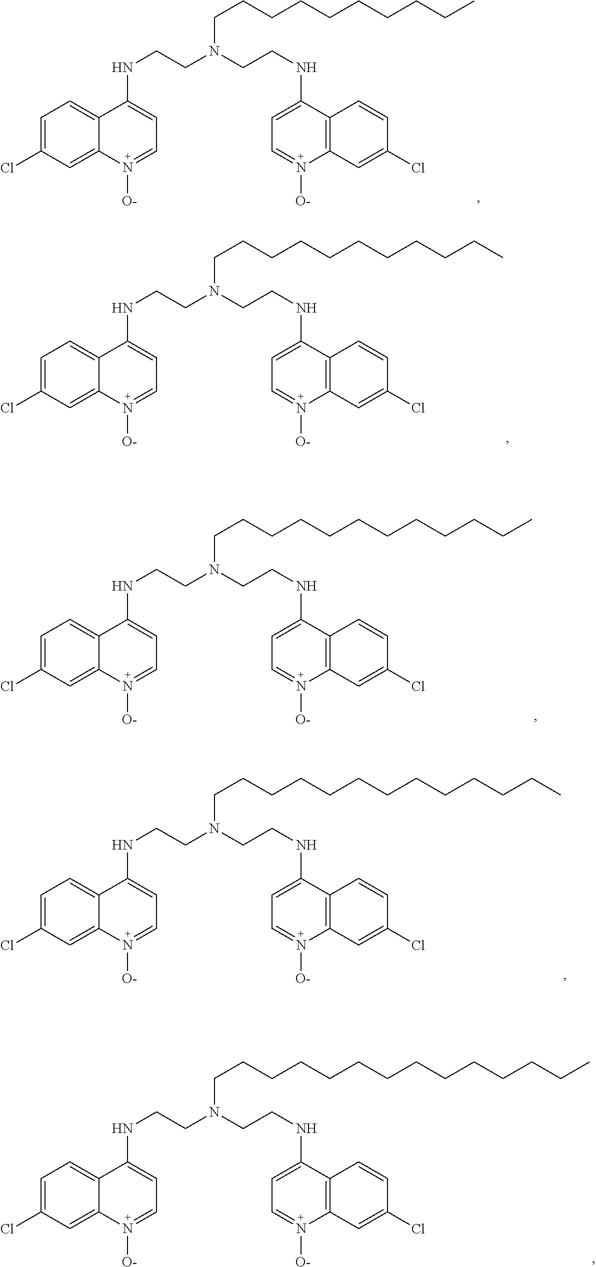

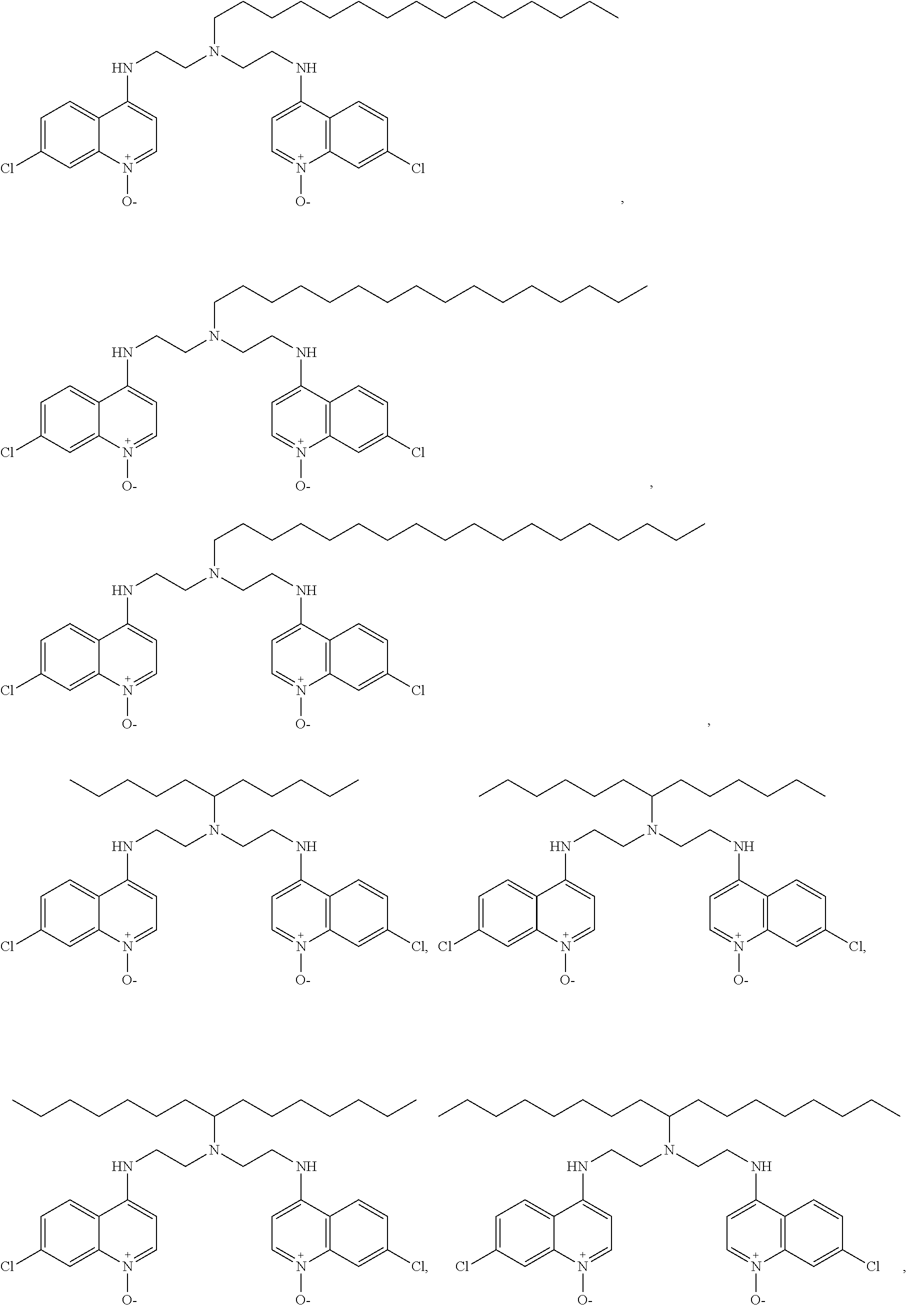

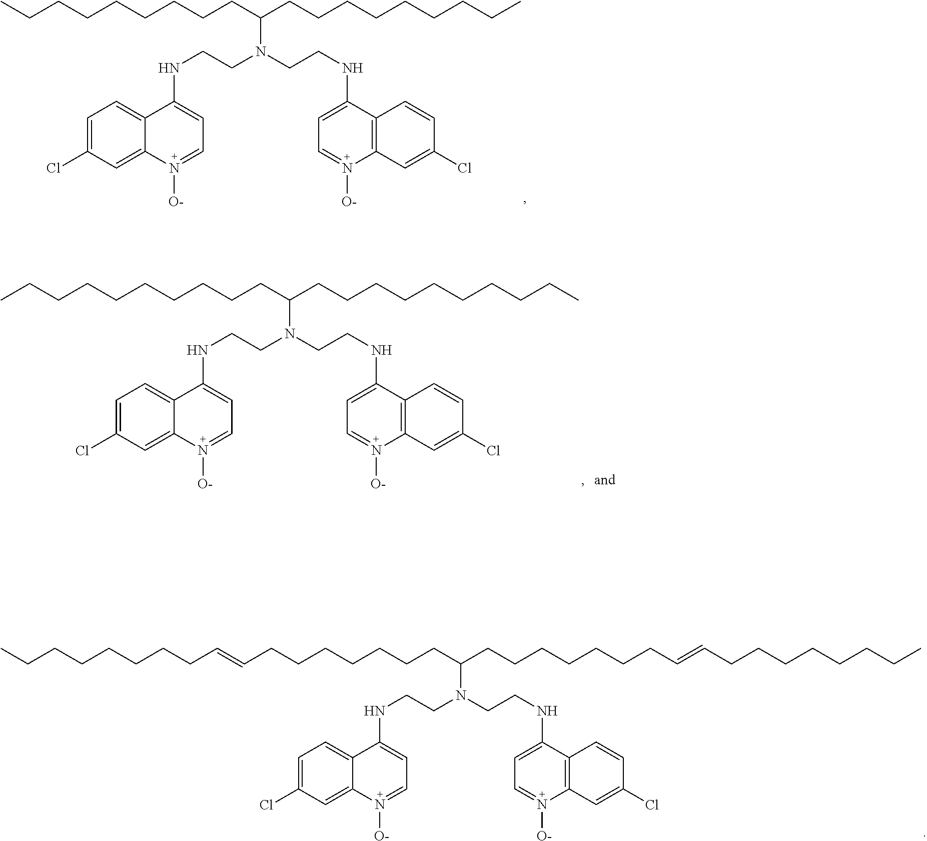

The present invention includes all tautomers and stereoisomers of compounds of the present invention, either in admixture or in pure or substantially pure form. The compounds of the present invention can have asymmetric centers at the carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers are within the scope of the present invention. Compounds according to the present invention can be prepared using diastereomers, enantiomers or racemic mixtures as starting materials. Furthermore, diastereomer and enantiomer products can be separated by chromatography, fractional crystallization or other methods known to those of skill in the art.

The present invention also includes isotopically-labeled compounds of the present invention, wherein one or more atoms are replaced by one or more atoms having specific atomic mass or mass numbers. Examples of isotopes that can be incorporated into compounds of the invention include, but are not limited to, isotopes of hydrogen, carbon, nitrogen, oxygen, fluorine, sulfur, and chlorine (such as ²H, ³H, ¹³C, C, ¹⁵N¹⁸O, ¹⁷O, ¹⁸F, ³⁵S and ³⁶Cl). Isotopically-labeled compounds of the present invention are useful in assays of the tissue distribution of the compounds and their prodrugs and metabolites; preferred isotopes for such assays include ³H and ¹⁴C. In addition, in certain circumstances substitution with heavier isotopes, such as deuterium (²H), can provide increased metabolic stability, which offers therapeutic advantages such as increased in vivo half-life or reduced dosage requirements. Isotopically-labeled compounds of this invention can generally be prepared according to the methods known by one of skill in the art by substituting an isotopically-labeled reagent for a non-isotopically labeled reagent. Compounds of the present invention can be isotopically labeled at positions adjacent to the basic amine, in aromatic rings, and the methyl groups of methoxy substituents.

The compounds of the present invention can also be in pharmaceutically acceptable salt forms, such as acid or base salts of the compounds of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

### IV. Nanocarrier

In some embodiments, the present invention provides a nanocarrier having an interior and an exterior, the nanocarrier comprising a plurality of compounds of the present invention, or a pharmaceutically acceptable salt thereof, wherein each compound self-assembles in an aqueous solvent to form the nanocarrier such that a hydrophobic pocket is formed in the interior of the nanocarrier, and a hydrophilic group self-assembles on the exterior of the nanocarrier.

The diameter of the nanocarrier of the present invention can be any suitable size. In some embodiments, the nanocarrier can have a diameter of 5 to 200 nm. In some embodiments, the nanocarrier can have a diameter of 10 to 150 nm. In some embodiments, the nanocarrier can have a diameter of 50 to 150 nm. In some embodiments, the nanocarrier can have a diameter of 100 to 150 nm. In some embodiments, the nanocarrier can have a diameter of about 80 nm, 90 nm, 100 nm, 110 nm, 120 nm, or 130 nm. In some embodiments, the nanocarrier can have a diameter of about 100 nm.

The exterior of the nanocarrier can be used for cell or lysosomal targeting. The nanocarrier can target the cell or lysosome to inhibit autophagy. In some embodiments, the nanocarriers can target lysosomal disruption, lysosomal dysfunctionl, autophagy inhibition, or a combination thereof. In some embodiments, the nanocarriers target the lysosome.

In some embodiments, the hydrophobic pocket is formed from the R¹ group of the compounds of the present invention. In some embodiments, the nanocarrier further comprises one or more hydrophobic drugs or imaging agents sequestered in the hydrophobic pocket of the nanocarrier.

The hydrophobic drugs useful in the present invention can be any hydrophobic drug known by one of skill in the art. Hydrophobic drugs useful in the present invention include, but are not limited to, deoxycholic acid, deoxycholate, resiquimod, gardiquimod, imiquimod, a taxane (e.g., paclitaxel, docetaxel, cabazitaxel, Baccatin III, 10-deacetylbaccatin, Hongdoushan A, Hongdoushan B, or Hongdoushan C), doxorubicin, etoposide, irinotecan, SN-38, cyclosporin A, podophyllotoxin, Carmustine, Amphotericin, Ixabepilone, Patupilone (epothelone class), rapamycin and platinum drugs. Other drugs includes non-steroidal anti-inflammatory drugs, and vinca alkaloids such as vinblastine and vincristine.

Other hydrophobic drugs useful in the present invention include, but are not limited to chemotherapeutic agents, molecular targeted agents, immunomodulatory agents, immunotherapeutic agents, a radiotherapeutic agents or a combination thereof.

In some embodiments, the hydrophobic drug is a chemotherapeutic agent, a molecular targeted agent, an immunotherapeutic agent, a radiotherapeutic agent or a combination thereof. In some embodiments, the hydrophobic drug is the immunotherapeutic agent. Immunotherapeutic agents useful in the present invention include, but are not limited to HCQ, Lys05, JQT, rapamycin, napabucasin, ipilimumab, nivolumab, pembrolizumab, atezolizumab, avelumab, and durvalumab.

In some embodiments, the hydrophobic drug is the radiotherapeutic agent. Radiotherapeutic agents useful in the present invention include, but are not limited to β-lapachone, cisplatin, nimorazole, cetuximab, misonidazole, and tirapazamine.

In some embodiments, the hydrophobic drug is the chemotherapeutic or molecular targeted agent. Chemotherapeutic or molecular targeted agents include, but are not limited to daunorubicin, doxorubicin, paclitaxel, docetaxel, abraxane, bortezomib, etoposide, lenalidomide, apoptozole, carboplatin, cisplatin, oxaliplatin, vinblastine, vincristine, trastuzumab, erlotinib, imatinib, nilotinib and vemurafenib.

In some embodiments, the hydrophobic drug is a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase (mek) inhibitor, a VEGF trap antibody, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR; INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated, estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH2 acetate [C₅₉H₈₄N₁₈O₁₄—(C₂H₄O₂)X where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegfilgrastim, erythropoietin, epoetin alfa and darbepoetin alfa, ipilumumab, vemurafenib or a combination thereof. In some embodiments, the hydrophobic drug is HCQ, Lys05, JQT, rapamycin, napabucasin, ipilimumab, nivolumab, pembrolizumab, atezolizumab, avelumab, durvalumab, β-lapachone, cisplatin, nimorazole, cetuximab, misonidazole, tirapazamine, daunorubicin, doxorubicin, paclitaxel, docetaxel, abraxane, bortezomib, etoposide, lenalidomide, apoptozole, carboplatin, cisplatin, oxaliplatin, vinblastine, vincristine, trastuzumab, erlotinib, imatinib, nilotinib, vemurafenib, or a combination thereof.

In some embodiments, the nanocarrier comprises a plurality of compounds of the present invention, with the compound structures as described above.

### V. Formulations & Administration

The compounds, nanocarriers and compositions of the present invention can be prepared in a wide variety of oral, parenteral and topical dosage forms. Oral preparations include tablets, pills, powder, dragee, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compound of the present invention.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the compound the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage).

Pharmaceutical preparations of the invention can also be used orally using, for example, push-fit capsules made of gelatin, as well as soft, sealed capsules made of gelatin and a coating such as glycerol or sorbitol. Push-fit capsules can contain the compound of the present invention mixed with a filler or binders such as lactose or starches, lubricants such as talc or magnesium stearate, and, optionally, stabilizers. In soft capsules, the compound of the present invention may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of the present invention is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997.

The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In another embodiment, the compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

The compositions of the present invention can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds and nanocarriers of the present invention can be present in any suitable amount, and can depend on various factors including, but not limited to, weight and age of the subject, state of the disease, etc. Suitable dosage ranges for the compound of the present invention include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds and nanocarriers the present invention can be administered at any suitable frequency, interval and duration. For example, the compound of the present invention can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with another active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, i.e., preparing a single pharmaceutical composition including both the compound of the present invention and the active agent. In other embodiments, the compound of the present invention and the active agent can be formulated separately.

The compound of the present invention and the active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w).

Other dosages and dosage ratios of the compound of the present invention and the active agent are suitable in the compositions and methods of the present invention.

### VI. Method of Treatment

In some embodiments, the present invention provides a method of treating a disease, comprising administering to a subject in need thereof, a therapeutically effective amount of a nanocarrier of the present invention.

In some embodiments, the method further comprises combination therapy by using additional agents for treating the disease. The additional agent is a therapeutic agent. Combination therapy of the present invention includes, but is not limited to, using a nanocarrier of the present invention, and one or more additional agent.

Combination therapy can include, but is not limited to immunotherapy, radiation therapy, chemotherapy, molecular targeted therapy, or a combination thereof.

In some embodiments, the method further comprises one or more additional agents, wherein the additional agent is a chemotherapeutic agent, a molecular targeted agent, an immunotherapeutic agent, a radiotherapeutic agent or a combination thereof. In some embodiments, the additional agent is the immunotherapeutic agent. Immunotherapeutic agents useful in the present invention are listed above. In some embodiments, the additional agent is the radiotherapeutic agent. Radiotherapeutic agents useful in the present invention are listed above. In some embodiments, the additional agent is the chemotherapeutic or molecular targeted agent. Chemotherapeutic and molecular targeted agents useful in the present invention are listed above.

In some embodiments, the one or more additional agents comprise two additional agents. In some embodiments, the additional agents are the immunotherapy agent and radiotherapeutic agent. In some embodiments, the additional agents are the immunotherapeutic agent and the chemotherapeutic agent. In some embodiments, the additional agents are the immunotherapeutic agent and molecular targeted agent. In some embodiments, the additional agents are the radiotherapeutic agent and chemotherapeutic agent. In some embodiments, the additional agents are the radiotherapeutic agent and molecular targeted agent.

In some embodiments, the additional agent is a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR; INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated, estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH2 acetate [C₅₉H₈₄N₁₈O₁₄—(C₂H₄O₂)X where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegfilgrastim, erythropoietin, epoetin alfa and darbepoetin alfa, ipilumumab, vemurafenib, or a combination thereof. In some embodiments, the additional agent is HCQ, Lys05, JQT, rapamycin, napabucasin, ipilimumab, nivolumab, pembrolizumab, atezolizumab, avelumab, durvalumab, β-lapachone, cisplatin, nimorazole, cetuximab, misonidazole, tirapazamine, daunorubicin, doxorubicin, paclitaxel, docetaxel, abraxane, bortezomib, etoposide, lenalidomide, apoptozole, carboplatin, cisplatin, oxaliplatin, vinblastine, vincristine, trastuzumab, erlotinib, imatinib, nilotinib, vemurafenib, or a combination thereof.

Diseases treated by the method of the present invention includes coronavirus, malaria, antiphospholipid antibody syndrome, lupus, rheumatiod arthritis, chronic urticaria or Sjogren's disease and cancer such as, but not limited to: carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, multiple myelomas, Hodgkin's lymphoma, and non-Hodgkin's lymphoma (see, CANCER: PRINCIPLES AND PRACTICE (DeVita, V. T. et al. eds 2008) for additional cancers).

Other diseases that can be treated by the nanocarriers of the present invention include: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; scleroderma; respiratory allergic diseases such as asthma, allergic rhinitis, hypersensitivity lung diseases, and the like, (2) autoimmune diseases, such as arthritis (rheumatoid and psoriatic), osteoarthritis, multiple sclerosis, systemic lupus erythematosus, diabetes mellitus, glomerulonephritis, and the like, (3) graft rejection (including allograft rejection and graft-v-host disease), and (4) other diseases in which undesired inflammatory responses are to be inhibited (e.g., atherosclerosis, myositis, neurological conditions such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis and Behcet's syndrome).

In some embodiments, the disease is cancer. In some embodiments, the cancer is bladder cancer, brain cancer, breast cancer, cervical cancer, cholangiocarcinoma, colorectal cancer, esophageal cancer, gall bladder cancer, gastric cancer, glioblastoma, intestinal cancer, head and neck cancer, leukemia, liver cancer, lung cancer, melanoma, myeloma, ovarian cancer, pancreatic cancer, prostate and uterine cancer. In some embodiments, the cancer is bladder cancer, brain cancer, breast cancer, cervical cancer, cholangiocarcinoma, colorectal cancer, esophageal cancer, gall bladder cancer, gastric cancer, glioblastoma, intestinal cancer, head and neck cancer, leukemia, liver cancer, lung cancer, melanoma, myeloma, ovarian cancer, pancreatic cancer and uterine cancer.

In some embodiments, the disease is coronavirus, malaria, antiphospholipid antibody syndrome, lupus, rheumatiod arthritis, chronic urticaria or Sjogren's disease.

In some embodiments, the method of treating the disease comprises targeting cell autophagy and/or the lysosome. Targeting autophagy can result in either autophagy inhibition or autophagy activation. Targeting the lysosome can result in lysosomal disruption, lysosomal dysfunction, or both.

In some embodiments, the method of treating targets lysosomal disruption, lysosomal dysfunction and/or autophagy inhibition. In some embodiments, the method of treating targets the lysosome.

In some embodiments, the nanocarrier targets lysosomal disruption, lysosomal dysfunction and/or autophagy inhibition. In some embodiments, the nanocarrier targets the lysosome.

### VII. Method of Imaging

In some embodiments, the present invention provides a method of imaging, comprising administering to a subject to be imaged, an effective amount of a nanocarrier of the present invention.

The imaging agents useful in the present invention can be any imaging agent known by one of skill in the art. Imaging agents include, but are not limited to, paramagnetic agents, optical probes, and radionuclides. Paramagnetic agents are imaging agents that are magnetic under an externally applied field. Examples of paramagnetic agents include, but are not limited to, iron particles including nanoparticles. Optical probes are fluorescent compounds that can be detected by excitation at one wavelength of radiation and detection at a second, different, wavelength of radiation. Optical probes useful in the present invention include, but are not limited to, Cy5.5, Alexa 680, Cy5, DiD (1,1′-dioctadecyl-3,3,3′,3′-tetramethylindodicarbocyanine perchlorate) and DiR (1,1′-dioctadecyl-3,3,3′,3′-tetramethylindotricarbocyanine iodide). Other optical probes include quantum dots.

Radionuclides are elements that undergo radioactive decay. Radionuclides useful in the present invention include, but are not limited to, ³H, ¹¹C, ¹³N, ¹⁸F, ¹⁹F, ⁶⁰Co, ⁶⁴Cu, ⁶⁷Cu, ⁶⁸Ga, ⁸²Rb, ⁹⁰Sr, ⁹⁰Y, ⁹⁹Tc, ^{99m}Tc, ¹¹¹In, ¹²³I, ¹²⁴I, ¹²⁵I, ¹²⁹I, ¹³¹I, ¹³⁷Cs, ¹⁷⁷Lu, ¹⁸⁶Re, ¹⁸⁸Re, ²¹¹At, Rn, Ra, Th, U, Pu and ²⁴¹Am.

Imaging methods useful in the present invention include, but are not limited to fluorescence microscopy, positron emission tomography (PET), magnetic resonance imaging (MRI), ultrasound, single photon emission computed tomography (SPECT), x-ray computed tomography (CT), echocardiography, and functional near-infrared spectroscopy.

### VIII. Examples

### Example 1: Compounds

Materials and instruments. Chemicals like Diethylenetriamine, Dodecyl aldehyde, fatty alcohol, pyridinium dichromate, sodium cyanoborohydride, ammonium hydroxide solution, deuterated solvents, anhydrous solvents, and Z-Arg-Arg-AMC were purchased from Millipore-Sigma (MO, USA). 4,7-Dichloroquinoline, anhydrous salt sulfate, and the bulk of solvents were purchased from Fisher Scientific (MA, USA). All solvents were used directly without further purification. Water used in all experiments was purified with a Mill-Q filtration system. Other reagents or drugs were purchased as indicated: tridecanal (Alfa Aesar), HCQ (Specturm), Lys05 (MedchemExpress), Bortezomib (eNovation chemical), DiD perchlorate and β-lapachone (Tocris Bioscience), JQ1 and Napabucasin (ApExBIO), rapamycin, paclitaxel and vinblastine (LC Laboratory), CN38 (Acros Organics), Etoposide (AdipoGen), Lenalidomide (Matrix Scientific), Napabucasin (ApExBIO) and Apoptozole (Selleck). Lysosome enrichment kit, LysoTracker (Red & Green), acridine orange, Dextran-Alexa Fluor 488, Premo™ Autophagy Sensor LC3B-GFP were bought from Thermo Fisher (MA, USA). The SensoLyte® homogeneous AMC caspase-3/7 assay kit and FITC-Annexin V/PI Apoptosis kit were bought from AnaSpec (CA, USA) and Biolegend (CA, USA), respectively. The compounds were characterized by a 600 MHz NMR spectrometer (Bruker, German) for NMR spectra and an LTQ-Orbitrap XL Hybrid ion trap mass spectrometer (Thermo Fisher, MA, USA) for ESI-HRMS spectra. Cell imaging studies were performed by a fluorescence microscope (Olympus, Tokyo, Japan) or a LSM800 confocal microscope (Carl Zeiss, Oberkochen, Germany). The absorbance and fluorescence intensity were determined with a SpectraMax M2 microplate reader (Molecular Devices, CA, USA). Western Blot was developed by a Power Pac 200 electrophoresis apparatus (Bio-Rad, CA, USA). The studies, including WB imaging, in vivo, and ex vivo fluorescence imaging, were performed on a ChemiDoc™ MP imaging system (Bio-Rad, CA, USA). DLS experiments were done with a Zetasizer Nano ZS (Malvern Instruments, Worcestershire, UK). TEM was performed on a Talos L120C TEM (FEI, OR, USA) with 80 kV acceleration voltage. Apoptosis assay was carried out by using a BD FACSCanto II flow cytometer (BD Biosciences, NJ, USA). Isolation of cancer stem cells was conducted by a BD FACSAria II Cell Sorter (BD Biosciences, NJ, USA). The Matrigel for 3D culture (Cat #354230) and xenograft model establishment (Cat #354234) were both purchased from Corning (NY, USA). LC3B antibody (1:1000, Catalog: #2775), SQSTM1/p62 antibody (1:1000, Catalog: #39749) and p-actin antibody (1:1000, Catalog: #4970) were purchased from Cell Signaling, and Pacific Blue anti-CD44 antibody (5 μL per million cells in 100 μL staining volume, Catalog: #338823); APC anti-CD326 (EpCAM) antibody (5 μL per million cells in 100 μL staining volume, Catalog: #324207); PE/Cy7 anti-CD24 antibody (5 μL per million cells in 100 μL staining volume, Catalog: #311119) were obtained from Biolegend.

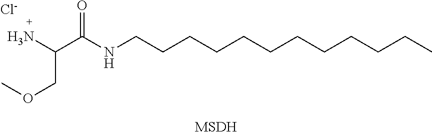 [SMILES: CCCCCCCCCCCCNC(=O)C(N)COC ; [Cl-]]

Synthesis of O-Methyl-Serine-Dodecylamide Hydrochloride (MSDH). MSDH was synthesized according to a published literature (Bioorg. Med. Chem. Lett. 1995, 5, 893-898). ¹H NMR (600 MHz, CDCl₃): δ 7.40 (s, 1H), 3.63 (m, 1H), 3.58 (m, 2H), 3.70 (s, 3H), 3.26 (m, 2H), 1.74 (s, 2H), 1.51 (m, 2H), 1.29 (m, 18H), 0.89 (t, 3H, J=7.2 Hz). ESI-HRMS: m/z [M+H]⁺ calcd for C₁₆H₃₅N₂O₂⁺ 287.2693, found 287.2690.

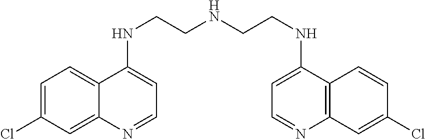 [SMILES: Clc1ccc2c(NCCNCCNc3ccnc4cc(Cl)ccc34)ccnc2c1]

Synthesis of BAQ. 4,7-dichloroquinoline (1.2 g, 6.00 mmol) in a 10 mL flask was maintained at 80° C. for 2 h without stirring, followed by adding diethylenetriamine (0.22 mL, 2.00 mmol). The reaction solution was stirred at 130° C. for 6 h. The residue was taken up with 30 mL methanol to afford white solid as the BAQ compound. Yield: 470 mg, 55%. ¹H NMR (600 MHz, DMSO-d₆): δ 8.38 (d, 2H, J=6.0 Hz), 8.23 (d, 2H, J=10.8 Hz), 7.78 (d, 2H, J=1.8 Hz), 7.42 (dd, 2H, J₁=10.8 Hz, J₂=2.4 Hz), 7.25 (s, 1H), 6.51 (d, 2H, J=6.6 Hz), 3.40 (t, 4H, J=7.2 Hz), 2.94 (t, 4H, J=7.8 Hz). ESI-HRMS: m/z [M+H]⁺ calcd for C₂₂H₂₂Cl₂N₅⁺ 426.1247, found 426.1243.

General synthetic method of BAQ12-18. To the solution of BAQ (426 mg, 1.0 mmol) in 30 mL anhydrous methanol and 10 mL anhydrous dichloromethane was added the corresponding aldehyde (2 mmol) and acetic acid (20 μL) and then was stirred for 20 min at room temperature, followed by adding sodium cyanoborohydride (126 mg, 2 mmol). The mixture was stirred for 12 h and was diluted by chloroform (100 mL). The organic phase was collected, washed with water, and dried by anhydrous sodium sulfate overnight. The crude product was purified via silica gel chromatography with the eluent containing 0.1% triethylamine (dichloromethane:methanol=30:1-10:1) to afford the corresponding compound.

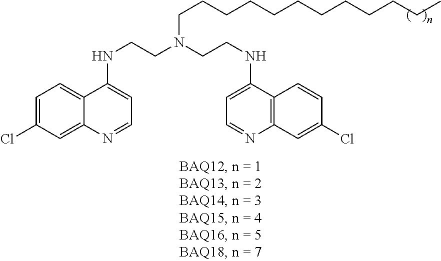

BAQ12. Yield: 320 mg, 53.8%. ¹H NMR (600 MHz, CD₃OD): δ 8.27 (d, 2H, J=5.4 Hz), 7.67 (d, 2H, J=2.4 Hz), 7.55 (d, 2H, J=9.0 Hz), 6.95 (dd, 2H, J₁=9.0 Hz, J₂=1.8 Hz), 6.46 (d, 2H, J=5.4 Hz), 3.41 (t, 4H, J=6.0 Hz), 2.90 (t, 4H, J=6.0 Hz), 2.66 (t, 2H, J=6.6 Hz), 1.55 (m, 2H), 1.33 (m, 20H), 0.91 (t, 3H, J=7.2 Hz). ¹³C NMR (150 MHz, CD₃OD): δ 150.9, 150.8, 147.9, 134.8, 126.3, 124.4, 121.9, 117.0, 98.4, 54.1, 51.9, 40.3, 31.6, 29.5, 29.5, 29.4, 29.4, 29.1, 27.4, 27.3, 22.4, 13.1. ESI-HRMS: m/z [M+H]⁺ calcd for C₃₄H₄₆Cl₂N₅⁺ 594.3125, found 594.3134.

BAQ13. Yield: 350 mg, 57.5%. ¹H NMR (600 MHz, CD₃OD): δ 8.27 (d, 2H, J=5.4 Hz), 7.67 (d, 2H, J=1.8 Hz), 7.56 (d, 2H, J=9.0 Hz), 6.96 (dd, 2H, J₁=9.0 Hz, J₂=2.4 Hz), 6.46 (d, 2H, J=5.4 Hz), 3.42 (t, 4H, J=6.0 Hz), 2.90 (t, 4H, J=6.0 Hz), 2.66 (t, 2H, J=7.2 Hz), 1.56 (m, 2H), 1.32 (m, 23H), 0.92 (t, 3H, J=7.2 Hz). ¹³CNMR (150 MHz, CD₃OD): δ 151.0, 150.7, 147.8, 134.8, 126.1, 124.5, 121.9, 117.0, 98.4, 54.6, 51.9, 40.3, 31.7, 29.5, 29.5, 29.5, 29.4, 29.1, 27.4, 27.3, 22.3, 13.1. ESI-HRMS: m/z [M+H]⁺ calcd for C₃₅H₄₈Cl₂N₅⁺ 608.3281, found 608.3274.

BAQ14. Yield: 295 mg, 47.4%. ¹H NMR (600 MHz, CD₃OD): δ 8.56 (d, 2H, J=9.0 Hz), 8.48 (d, 2H, J=4.8 Hz), 7.86 (d, 2H, J=1.2 Hz), 7.60 (dd, 2H, J₁=9.0 Hz, J₂=1.2 Hz), 7.06 (d, 2H, J=5.4 Hz), 4.16 (s, 4H), 3.82 (s, 4H), 3.48 (s, 2H), 1.92 (s, 2H), 1.44 (s, 2H), 1.35 (m, 23H), 0.93 (t, 3H, J=6.6 Hz). ¹³C NMR (150 MHz, CD₃OD): δ 155.9, 143.0, 139.8, 138.2, 127.4, 125.3, 118.7, 115.5, 98.9, 54.6, 51.1, 38.3, 31.5, 29.2, 29.2, 29.2, 29.2, 29.1, 29.0, 28.9, 28.7, 26.1, 23.0, 22.2, 12.9. ESI-HRMS: m/z [M+H]⁺ calcd for C₃₆H₅₀Cl₂N₅⁺ 622.3438, found 622.3505.

BAQ15. Yield: 290 mg, 45.5%. ¹H NMR (600 MHz, CD₃OD): δ 8.56 (d, 2H, J=9.0 Hz), 8.48 (d, 2H, J=6.0 Hz), 7.86 (d, 2H, J=1.2 Hz), 7.59 (dd, 2H, J₁=9.0 Hz, J₂=1.2 Hz), 7.07 (d, 2H, J=6.0 Hz), 4.16 (s, 4H), 3.82 (s, 4H), 3.48 (s, 2H), 1.92 (s, 2H), 1.44 (s, 2H), 1.35 (m, 25H), 0.93 (t, 3H, J=6.6 Hz). ¹³C NMR (150 MHz, CD₃OD): δ 155.8, 143.0, 139.7, 138.1, 127.3, 125.3, 118.7, 115.4, 98.9, 54.5, 51.0, 38.2, 31.5, 29.2, 29.2, 29.2, 29.1, 29.0, 28.9, 28.7, 26.1, 23.0, 22.2, 12.9. ESI-HRMS: m/z [M+H]⁺ calcd for C₃₇H₅₂Cl₂N₅⁺ 636.3594, found 636.3661.

BAQ16. Yield: 280 mg, 43.0%. ¹H NMR (600 MHz, CD₃OD): δ 8.56 (d, 2H, J=9.0 Hz), 8.48 (d, 2H, J=6.6 Hz), 7.86 (d, 2H, J=1.8 Hz), 7.59 (dd, 2H, J₁=9.0 Hz, J₂=1.8 Hz), 7.07 (d, 2H, J=7.2 Hz), 4.17 (m, 4H), 3.84 (m, 4H), 3.50 (t, 2H, J=7.8 Hz), 1.92 (m, 2H), 1.44 (m, 2H), 1.30 (m, 27H), 0.93 (t, 3H, J=7.2 Hz). ¹³C NMR (150 MHz, CD₃OD): δ 155.9, 143.0, 139.7, 138.1, 127.3, 125.2, 118.7, 115.4, 98.8, 54.5, 51.0, 38.2, 31.5, 29.2, 29.2, 29.2, 29.1, 28.9, 28.9, 28.7, 26.1, 23.0, 22.1, 12.9. ESI-HRMS: m/z [M+H]⁺ calcd for C₃₈H₅₄Cl₂N₅⁺ 650.3751, found 650.3774.

BAQ18. Yield: 290 mg, 42.7%. ¹H NMR (600 MHz, CD₃OD): δ 8.56 (d, 2H, J=9.0 Hz), 8.48 (d, 2H, J=5.4 Hz), 7.86 (s, 2H, J=1.8 Hz), 7.56 (d, 2H, J₁=8.4 Hz), 7.06 (d, 2H, J=6.6 Hz), 4.16 (s, 4H), 3.82 (m, 4H), 3.48 (s, 2H), 1.91 (s, 2H), 1.43 (m, 2H), 1.30 (m, 29H), 0.93 (s, 3H). ¹³C NMR (150 MHz, CD₃OD): δ 155.9, 143.0, 139.8, 138.2, 127.4, 125.2, 118.7, 115.5, 98.8, 54.5, 51.1, 38.2, 31.5, 29.2, 29.1, 28.9, 28.9, 28.7, 26.1, 23.0, 22.2, 12.9. ESI-HRMS: m/z [M+H]⁺ calcd for C₃₈H₅₄Cl₂N₅⁺ 650.3751, found 650.3774. ESI-HRMS: m/z [M+H]⁺ calcd for C₄₀H₅₆Cl₂N₅⁺ 678.4064, found 678.4069.

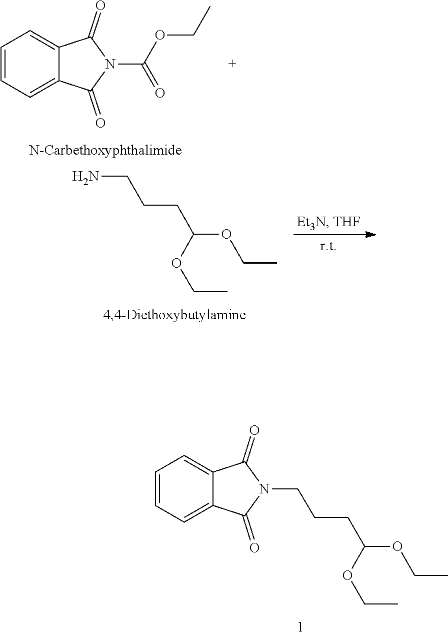 [SMILES: CCOC(=O)N1C(=O)c2ccccc2C1=O ; "+" ; CCOC(CCCN)OCC ; CCOC(CCCN1C(=O)c2ccccc2C1=O)OCC]

Synthesis of Compound 1. To the solution of 4,4-diethoxybutylamine (1.84 mL, 10.7 mmol, 1.00 eq) in THF (30 mL) were added ethyl N-carbethoxyphthalimide (2.34 g, 10.7 mmol, 1.00 eq) and triethylamine (1.49 mL, 10.7 mmol, 1.00 eq). The resulting reaction mixture was stirred at room temperature for 12 h. After removing the solvent under reduced pressure, the resulting crude material was purified on a silica column eluting with 1:20 EtOAc to hexanes to yield a clear oil. (2.9 g, 94.0 mmol, 93% yield). ¹H NMR (600 MHz, DMSO-d₆) δ 7.87-7.83 (m, 4H), 4.47 (t, J=5.4 Hz, 1H), 3.58-3.51 (m, 4H), 3.43-3.38 (m, 2H), 1.61-1.60 (m, 2H), 1.54-1.51 (m, 2H). ESI-HRMS m/z 314.1361 [M+Na]+.

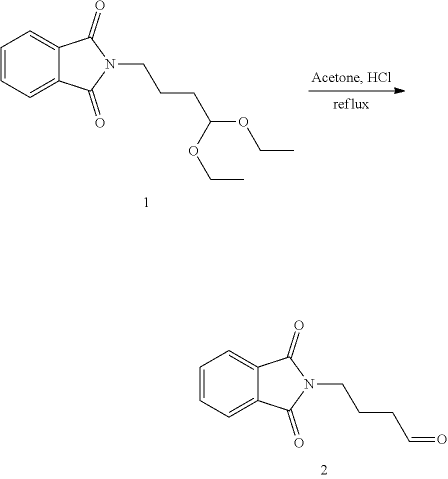 [SMILES: CCOC(CCCN1C(=O)c2ccccc2C1=O)OCC ; O=CCCCN1C(=O)c2ccccc2C1=O]

Synthesis of Compound 2. The solution of compound 1 (2.2 g, 7.6 mmol, 1.00 eq) in acetone (20 mL) and 1 M aqueous HCl (15.2 mL, 15.2 mmol, 2.00 eq) was stirred vigorously at reflux (80° C.) for 1 h. The acetone was evaporated under reduced pressure and the resulting aqueous layer was extracted 3 times with Et2O. Combined organic layers were washed once with water, dried over anhydrous sodium sulfate, filtered, and evaporated under reduced pressure, purified via column chromatography eluting with 1:2 EtOAc to hexanes to yield a waxy white solid. (1.2 g, 74% yield). ¹H NMR (600 MHz, DMSO-d₆) δ 9.64 (t, J=1.2 Hz, 1H, NH₂), 7.88-7.83 (m, 4H), 3.61 (t, J=7.2 Hz, 2H), 2.54-2.51 (m, 2H), 1.85-1.83 (m, 2H).

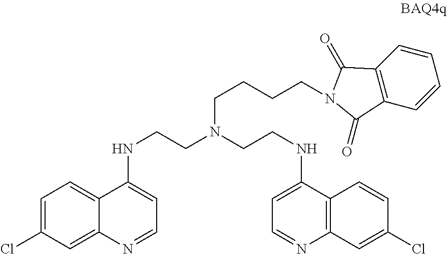 [SMILES: O=C1c2ccccc2C(=O)N1CCCCN(CCNc1ccnc2cc(Cl)ccc12)CCNc1ccnc2cc(Cl)ccc12]

Synthesis of BAQ4q. To the solution of BAQ (426.5 mg, 1.0 mmol, 1.00 eq) in methanol (40 mL) was added Compound 2 (434 mg, 2 mmol, 2.0 eq) and acetic acid (10 μL), which was stirred for 30 min at room temperature. Sodium cyanoborohydride (126 mg, 2 mmol, 2 eq) was added slowly and was stirred for 24 h. The reaction solution was diluted with dichloromethane (150 mL), then was washed by saturated sodium carbonate, water and brine, dried by anhydrous sodium sulfate. After filtration and concentrated under reduced pressure, the mixture was purified by silica chromatograph eluting with 20:1 dichloromethane to methanol to yield a white solid. (520 mg, 83% yield). ¹H NMR (600 MHz, DMSO-d₆) δ 8.27 (d, J=5.4 Hz, 2H), 7.97 (d, J=9.0 Hz, 2H), 7.78 (s, 4H), 7.67 (d, J=2.4 Hz, 2H), 7.19 (dd, J₁=9.0 Hz, J₂=2.4 Hz, 2H), 7.09 (m, 2H), 6.39 (d, J=6.0 Hz, 2H), 3.51 (t, J=7.2 Hz, 2H), 3.31 (t, J=6.0 Hz, 4H), 2.77 (t, J=6.6 Hz, 4H), 2.58 (t, J=6.6 Hz, 2H), 1.60-1.55 (m, 2H), 1.45-1.40 (m, 2H). ESI-HRMS 627.2035 [M+H]⁺.

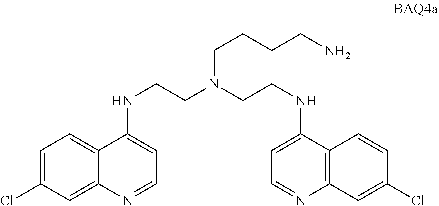 [SMILES: NCCCCN(CCNc1ccnc2cc(Cl)ccc12)CCNc1ccnc2cc(Cl)ccc12]

Synthesis of BAQ4a. BAQ4q (314 mg, 0.5 mmol, 1.0 eq) was dissolved in ethanol, and after adding hydrazine (2.5 mmol, 5.0 eq), the reaction solution was stirred for 12 h at reflux. Then the reaction mixture was allowed to be room temperature. After removing the precipitation by filtration, the filtrate was concentrated and then was added into ether (100 mL) to generate white precipitation, which was collected as Compound 5. (200 mg, 80% yield). ¹H NMR 8.30 (d, J=5.4 Hz, 2H), 8.03 (d, J=9.6 Hz, 2H), 7.78 (s, 4H), 7.71 (d, J=2.4 Hz, 2H), 7.27 (dd, J₁=9.0 Hz, J₂=2.4 Hz, 2H), 7.05 (t, J=5.4 Hz, 2H), 6.40 (d, J=5.4 Hz, 2H), 3.31 (t, J=6.6 Hz, 2H), 3.31 (t, J=6.0 Hz, 4H), 2.78 (t, J=7.2 Hz, 4H), 2.54 (t, J=7.2 Hz, 2H), 2.42 (t, J=7.2 Hz, 2H), 1.41-1.38 (m, 2H), 1.29-1.26 (m, 2H). ESI-HRMS 497.1981 [M+H]⁺.

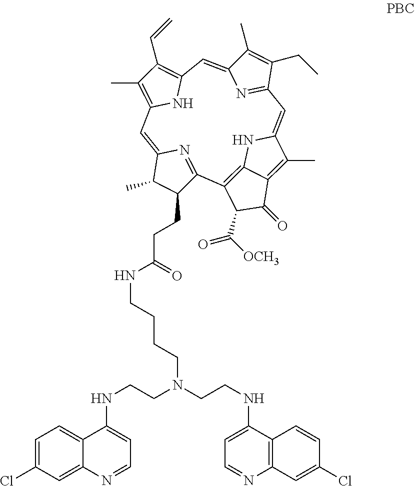 [SMILES: C=Cc1c(C)c2cc3nc(c4c5[nH]c(cc6nc(cc1[nH]2)C(C)=C6CC)c(C)c5C(=O)[C@@H]4C(=O)OC)[C@@H](CCC(=O)NCCCCN(CCNc1ccnc2cc(Cl)ccc12)CCNc1ccnc2cc(Cl)ccc12)[C@@H]3C]

Synthesis of PBC. Pheophorbide a (296 mg, 0.5 mmol, 1.0 eq), 6-Chloro-1-hydroxybenzotriazole (102 mg, 0.6 mmol, 1.2 eq), 1-Ethyl-3-(3-dimethylaminopropyl)carbodiimide (93 mg, 0.6 mmol, 1.2 eq) and N,N-diisopropylethylamine (174 μL, 1.0 mmol, 2 eq) was suspending in anhydrous dichloromethane (75 mL) and was stirred for 30 min at room temperature. Compound 5 (314 mg, 0.5 mmol, 1.0 eq) was added into the reaction mixture, which was then stirred for 48 h.

The mixture was purified by silica chromatograph eluting with 20:1 dichloromethane to methanol to yield a black solid. (200 mg, 37% yield). ¹H NMR (600 MHz, CD₃OD) δ 8.94 (s, 1H), 8.78 (s, 1H), 8.58 (s, 1H), 7.86 (dd, J₁=6.0 Hz, J₂=3.6 Hz, 2H), 7.86 (dd, J₁=6.0 Hz, J₂=3.6 Hz, 2H), 7.81 (dd, J₁=18.0 Hz, J₂=11.4 Hz, 2H), 7.63 (m, 1H), 7.54 (d, J=6.0 Hz, 2H), 7.18 (dd, J₁=9.0 Hz, J₂=2.4 Hz, 1H), 6.88 (m, 2H), 6.55 (d, J=8.4 Hz, 2H), 6.22 (dd, J₁=8.4 Hz, J₂=1.8 Hz, 2H), 6.17 (d, J=18.0 Hz, 1H), 6.10 (d, J=11.4 Hz, 1H), 5.55 (d, J=18.0 Hz, 1H), 4.52-4.51 (m, 1H), 4.15-4.14 (m, 1H), 3.88 (s, 3H), 3.72-3.67 (m, 2H), 3.30 (s, 3H), 3.2 (q, J₂=7.2 Hz, 4H, triethylamine), 2.89 (m, 1H), 2.80 (s, 3H), 2.75 (m, 1H), 2.63-2.61 (m, 2H), 2.56-2.53 (m, 1H), 2.50-2.43 (m, 4H), 2.3-2.16 (m, 8H), 1.94 (s, 1H), 1.82 (d, J=7.2 Hz, 3H), 1.47 (t, J=7.8 Hz, 3H), 1.35 (m, 10H+6H triethylamine), 1.11 (m, 4H). ESI-HRMS 1071.4576 [M+H]⁺.

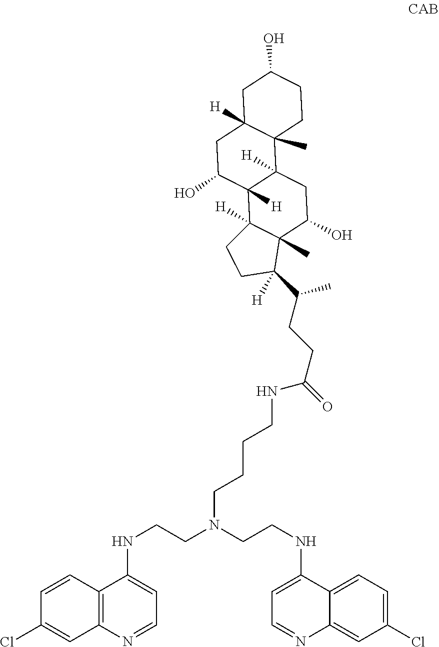

Synthesis of CAB. Cholic acid (204 mg, 0.5 mmol, 1.0 eq), 6-Chloro-1-hydroxybenzotriazole (102 mg, 0.6 mmol, 1.2 eq), 1-Ethyl-3-(3-dimethylaminopropyl)carbodiimide (93 mg, 0.6 mmol, 1.2 eq) and N,N-diisopropylethylamine (174 μL, 1.0 mmol, 2 eq) was suspending in anhydrous dichloromethane (75 mL) and was stirred for 30 min at room temperature. BAQ4a (314 mg, 0.5 mmol, 1.0 eq) was added into the reaction mixture, which was then stirred for 48 h. The mixture was purified by silica chromatograph eluting with 20:1 dichloromethane to methanol to yield a black solid. (220 mg, 49.5% yield). ESI-HRMS 887.4774 [M+H]⁺.

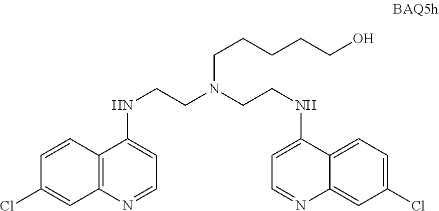 [SMILES: OCCCCCN(CCNc1ccnc2cc(Cl)ccc12)CCNc1ccnc2cc(Cl)ccc12]

Synthesis of BAQ5h. To the solution of BAQ (426.5 mg, 1.0 mmol, 1.00 eq) in methanol (40 mL) were added Glutaric dialdehyde (211 μL, 2.0 mmol, 2.00 eq) and acetic acid (10 L), which was stirred for 30 min at room temperature. Sodium cyanoborohydride (126 mg, 2 mmol, 2 eq) was added slowly and was stirred for 24 h. The reaction solution was diluted with dichloromethane (150 mL), then was washed by saturated sodium carbonate, water and brine, dried by anhydrous sodium sulfate. After filtration and concentrated under reduced pressure, the mixture was purified by silica chromatograph eluting with 20:1 dichloromethane to methanol to yield a white solid. (520 mg, 83% yield). ¹H NMR (600 MHz, CD₃OD) S 8.23 (d, J=5.4 Hz, 2H), 7.60-7.56 (m, 4H), 7.02 (dd, J₁=9.0 Hz, J₂=1.8 Hz, 2H), 6.44 (t, J=5.4 Hz, 2H), 3.58 (t, J=6.6 Hz, 2H), 3.42 (t, J=5.4 Hz, 4H), 2.91 (t, J=6.0 Hz, 4H), 2.75-2.72 (m, 2H), 1.66-1.57 (m, 4H), 1.51-1.47 (m, 2H). ESI-HRMS 512.1961 [M+H]⁺.

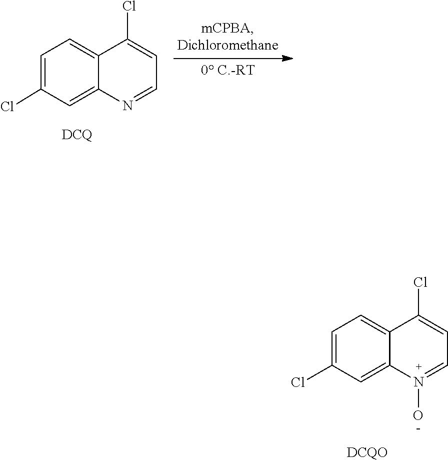

Synthesis of DCQO. 4,7-dichloroquinoline (DCQ) (2 g, 10 mmol) was dissolved in 50 mL and was vigorously stirred (500 rmp) on an ice-water bath for 15 min. mCPBA (2.7 g, 12 mmol) was added carefully in bath (4 times) to the reaction solution. The resulting reaction mixture was allowed to stir (500 rpm) at room temperature for 12 hr. TLC indicated complete conversion of starting materials to one major spot. To the reaction solution was added dichloromethane (100 mL) and potassium carbonate (4.1 g, 30 mmol), which was stirred (300 rpm) for 1 hr at room temperature. The mixture was poured into a 500 mL baker with 200 mL water and stirred (300 rpm) for another 1 hr. (The organic phase was collected, washed with saturated sodium carbonate (75 mL×3), water (75 mL×3), brine (75 mL×3), respectively, and dried with anhydrous sodium sulfate overnight. After filtration, solvent was evaporated under reduced pressure, and the resulting crude material was recrystallized with 80 mL acetonitrile. The resulting solid product was filtered for collection, and then was dried under vacuum to afford 1.8 g DCQO as white solid. ¹HNMR (600 MHz, CDCl₃) δ 8.79 (d, J=1.8 Hz, 2H), 8.44 (d, J=6.6 Hz, 2H), 8.44 (d, J=8.4 Hz, 2H), 7.71 (d, J₁=9.0 Hz, J₂=2.4 Hz, 2H), 7.38 (d, J=6.0 Hz, 2H). HRMS (ESI): m/z calcd for C₉H₆Cl₂NO [M+H]+ 213.9821, found 213.9834.

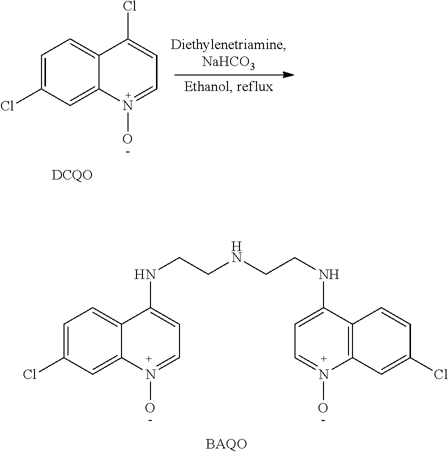

Synthesis of BAQO. To the solution of DCQO (2.14 g, 10 mmol) in 30 mL anhydrous ethanol was added sodium bicarbonate (840 mg, 10 mmol) and diethylenetriamine (432 μL, 4 mmol). The mixture was refluxed at 95° C. for 48 hr. TLC was used to indicate the generation of the target materials material (TM, the yellow spot). Ethanol was evaporated under reduced pressure and the residue was re-resuspended by 30 mL methanol, which was slowly dropped into a 300 mL baker with the mixed solution of 100 mL hydrochloric acid (1 M) and 50 mL dichloromethane. The aqueous phase was collected, washed with dichloromethane (50 mL×2), alkalized to pH 10 using 10 M NaOH (12 mL) to generate yellow precipitation. The precipitation was collected, washed by water (30 mL×3) and dried under vacuum to afford 850 mg BAQO as yellow solid. ¹HNMR (600 MHz, CD₃OD) S 8.51 (d, J=1.8 Hz, 2H), 8.35 (d, J=7.2 Hz, 2H), 8.14 (d, J=9.0 Hz, 2H), 7.55 (d, J₁=9.0 Hz, J₂=2.4 Hz, 2H), 6.63 (d, J=7.2 Hz, 2H), 3.58 (t, J=6.0 Hz, 4H), 2.94 (t, J=6.0 Hz, 4H). HRMS (ESI): m/z calcd for C₂₂H₂₂Cl₂N₅O₂ [M+H]⁺ 458.1145, found 458.1126.

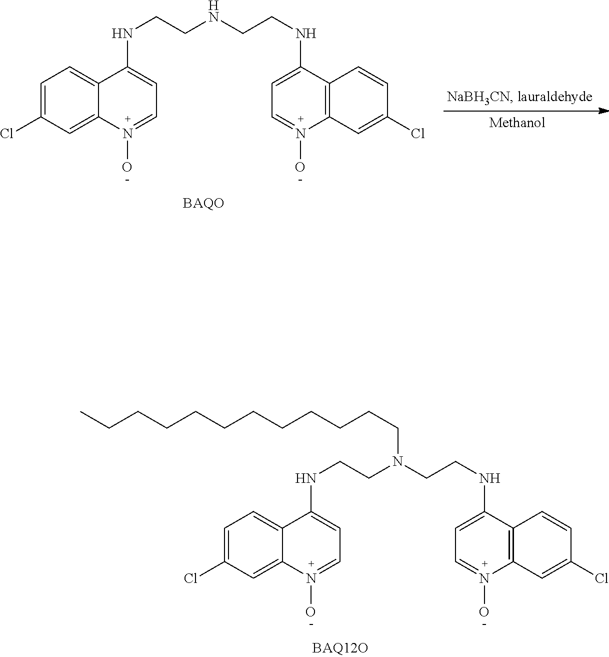

Synthesis of BAQ120. The mixture of BAQO (916 mg, 2 mmol), dodecyl aldehyde (1.8 mL, 8 mmol) and acetic acid (20 μL) was vigorously stirred (500 rpm) at room temperature for 30 min. Sodium cyanoborohydride (377 mg, 6 mmol) was then added slowly.

The reaction mixture was stirring at room temperature for another 12 hrs. TLC indicated the complete conversion of starting materials to one major spot. The solvent was concentrated to 25 mL and then the resulting residue was diluted by 75 ml dichloromethane). The organic phase was washed with 100 mL saturated sodium bicarbonate three times. The emulsion layer was collected, and then was filtered to provide a yellow solid, which was washed by water (30 mL×3) and ethyl ether (30 mL×3). The collected yellow solid was dried under vacuum to afford 1.2 g BAQ120. ¹HNMR (CD3OD, 600 MHz) δ 8.43 (d, J=1.8 Hz, 2H), 8.31 (d, J=7.2 Hz, 2H), 7.82 (d, J=9.0 Hz, 2H), 7.31 (d, J₁=9.0 Hz, J₂=2.4 Hz, 2H), 6.56 (d, J=7.2 Hz, 2H), 3.50 (t, J=6.0 Hz, 4H), 2.94 (t, J=6.0 Hz, 4H), 2.71 (t, J=7.2 Hz, 2H), 1.55 (m, 2H), 1.32 (m, 18H), 0.92 (t, J₁=6.6 Hz, 3H). CNMR (CD₃OD, 150 MHz) δ 148.0, 140.1, 139.9, 138.4, 127.6, 123.9, 118.7, 117.9, 98.09, 54.9, 52.3, 41.5, 32.4, 30.2, 30.1, 30.1, 29.8, 28.1, 27.7, 23.1, 13.8. HRMS (ESI): m/z calcd for C₃₄H₄₆Cl₂N₅O₂ [M+H]+ 626.3023, found 626.3060.

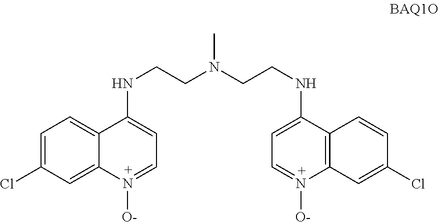

Synthesis of BAQ10. The compound was prepared using the method of BAQ120. Formaldehyde was used as a starting material. ESI-HRMS 472.1325 [M+H]⁺.

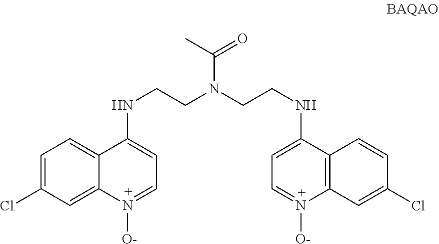

Synthesis of BAQAO. BAQO (229 mg, 0.5 mmol) in 5 mL acetic anhydride was refluxed for 6 h. The excessive acetic anhydride was removed under reduced pressure. The residue was taken up with cold diethyl ether to afford the yellow solid as the product (180 mg). ESI-HRMS 500.1246 [M+H]⁺.

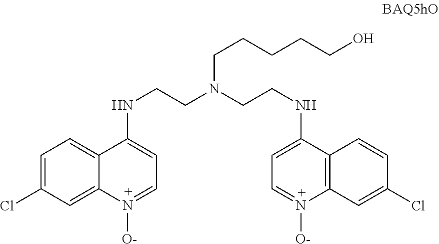

Synthesis of BAQ5hO. The compound was prepared using the method of BAQ5h. BAQO was used as a starting material. ESI-HRMS 544.1870 [M+H]⁺.

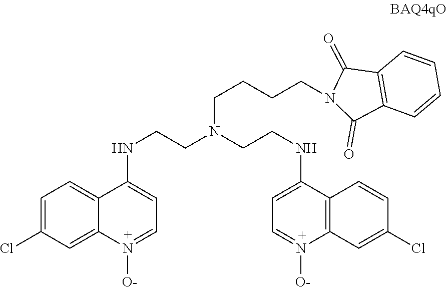

Synthesis of BAQ4qO. The compound was prepared using the method of BAQ4q. BAQO was used as a starting material. ESI-HRMS 659.1946 [M+H]⁺.

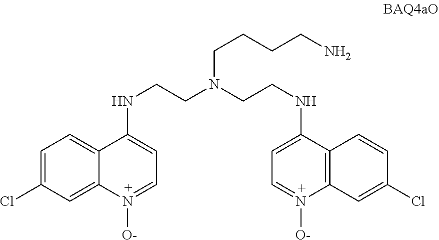

Synthesis of BAQ4aO. The compound was prepared using the method of BAQ4a. BAQ4qO was used as a starting material. ESI-HRMS 529.1884 [M+H]⁺.

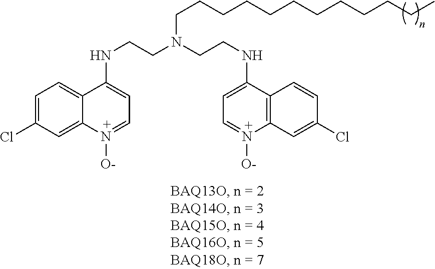

Synthesis of BAQ130. The compound was prepared using the method of BAQ12O. Tridecanal was used as a starting material. ¹HNMR (800 MHz, DMSO-d₆) δ 9.79 (s, 2H), 8.91 (d, J=7.2 Hz, 2H), 8.78 (d, J=9.6 Hz, 2H), 8.16 (d, J=1.6 Hz, 2H), 7.80 (dd, J₁=9.6 Hz, J₁=1.8 Hz, 2H), 6.97 (d, J=8.0 Hz, 2H), 4.04 (s, 4H), 3.68-3.62 (m, 6H), 1.70 (s, 2H), 1.27-1.17 (m, 21H), 0.87 (t, J=7.2 Hz, 3H).

Synthesis of BAQ140. The compound was prepared using the method of BAQ120. Tetradecanal was used as a starting material. ¹HNMR (800 MHz, DMSO-d₆) δ 9.62 (s, 2H), 8.88 (d, J=8.0 Hz, 2H), 8.70 (d, J=9.6 Hz, 2H), 8.15 (d, J=2.4 Hz, 2H), 7.77 (dd, J₁=8.8 Hz, J₁=1.6 Hz, 2H), 6.95 (d, J=7.2 Hz, 2H), 4.02 (s, 4H), 3.68-3.62 (m, 6H), 1.68 (s, 2H), 1.27-1.15 (m, 23H), 0.86 (t, J=5.4 Hz, 3H).

Synthesis of BAQ150. The compound was prepared using the method of BAQ120. Pentadecanal was used as a starting material. ¹HNMR (800 MHz, DMSO-d₆) δ 9.63 (s, 2H), 8.88 (d, J=8.0 Hz, 2H), 8.70 (d, J=8.8 Hz, 2H), 8.15 (d, J=2.4 Hz, 2H), 7.77 (dd, J₁=8.8 Hz, J₁=1.6 Hz, 2H), 6.95 (d, J=8.8 Hz, 2H), 4.01 (s, 4H), 3.68-3.62 (m, 6H), 1.68 (s, 2H), 1.28-1.15 (m, 25H), 0.86 (t, J=5.4 Hz, 3H).

Synthesis of BAQ160 & BAQ180. The compound was prepared using the method of BAQ120. Hexadecanal was used as a starting material for BAQ160 and octadecanal was used as starting material for BAQ180.

Additional BAQO derivatives can be prepared using the method of BAQ120 using appropriate aldehyde and ketone starting materials.

### Example 2: Nanocarriers

Preparation and characterization of BAQ ONNs. NPs were prepared through the re-precipitation method. BAQ derivatives in methanol were added dropwise into MilliQ water while stirring for 5 min (volume ratio, 1:10), and then homogenous NPs were obtained after rotary evaporation (40° C., 20 min), followed by the characterization with Zetasizer Nano ZS (Malvern). The TEM samples were prepared by dropping 0.5 mM NPs on carbon square mesh and dried naturally, which were then observed under the Talos L120C TEM (FEI) at an accelerating voltage of 80 kV. To determine the drug content in nanoformulations, the prepared drug-loaded NPs were cut off by centrifugal filter (ultracel-10 kDa, Millipore), and the absorbance of filtrate (diluted with DMSO, 1:10, volume ratio) was measured for calculation of drug concentrations.

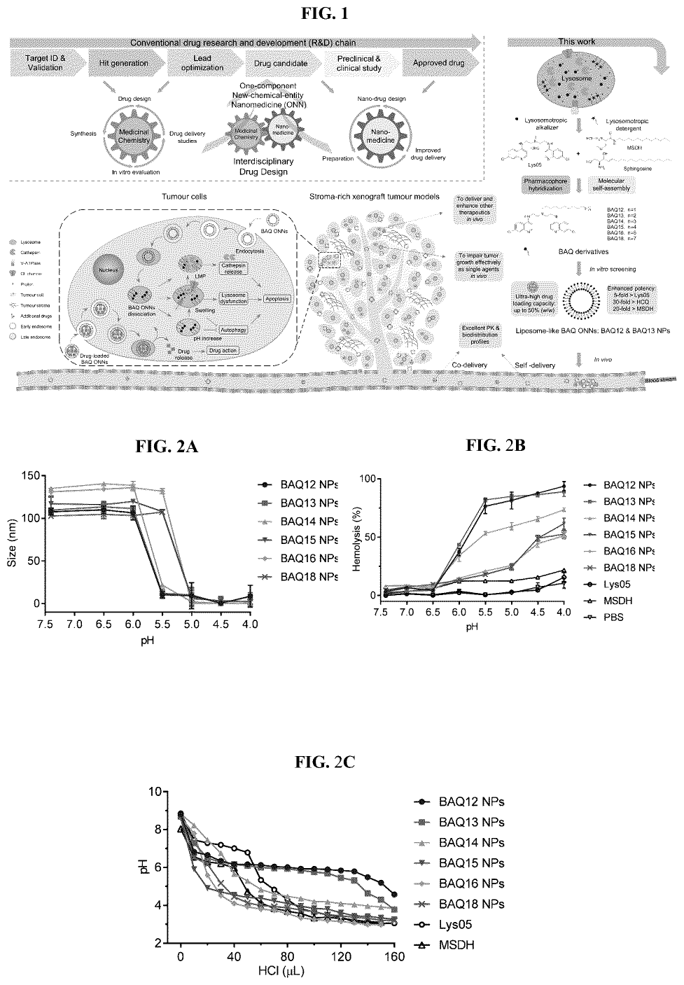

Discovery of BAQ derivatives as potential ONNs. BAQ12-BAQ18 were designed via hybridization of the key structural elements of the lysosomotropic autophagy inhibitor Lys05 and the lysosomotropic detergent MSDH to achieve pharmacological fusion (FIG. 1). Based on self-assembly principles, it was envisioned that the inclusion of long hydrophobic tails with the cationic BAQ heads would drive them to form nanoparticles (NPs). Since BAQ heads have a calculated pKa of 8.4, this self-assembly should be dependent on the surroundings' pH, wherein NPs are formed under neutral conditions and are dissociated into free building blocks after protonation in acidic environments.

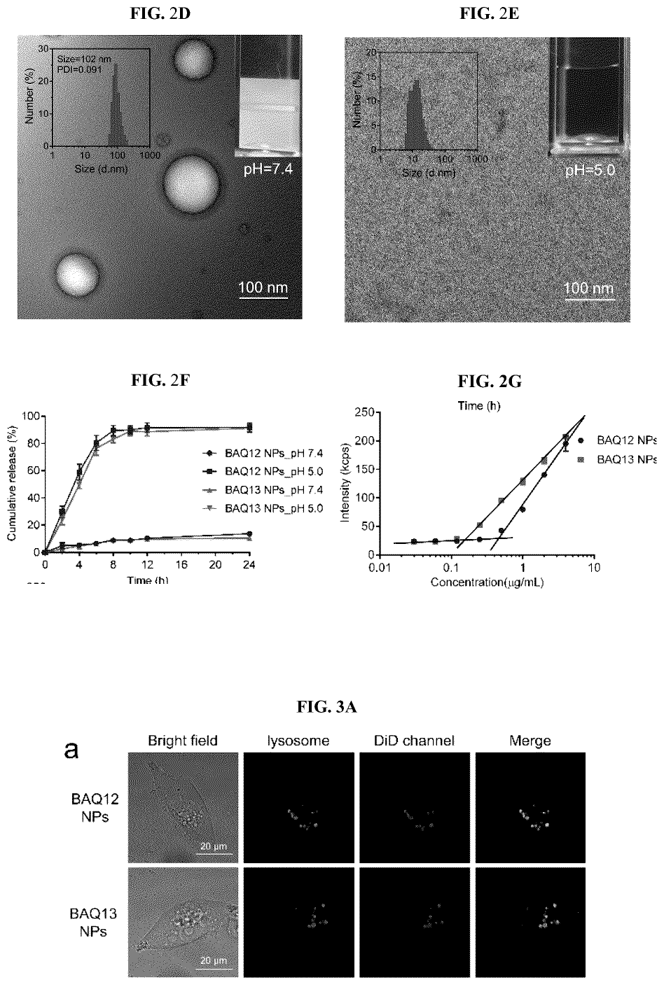

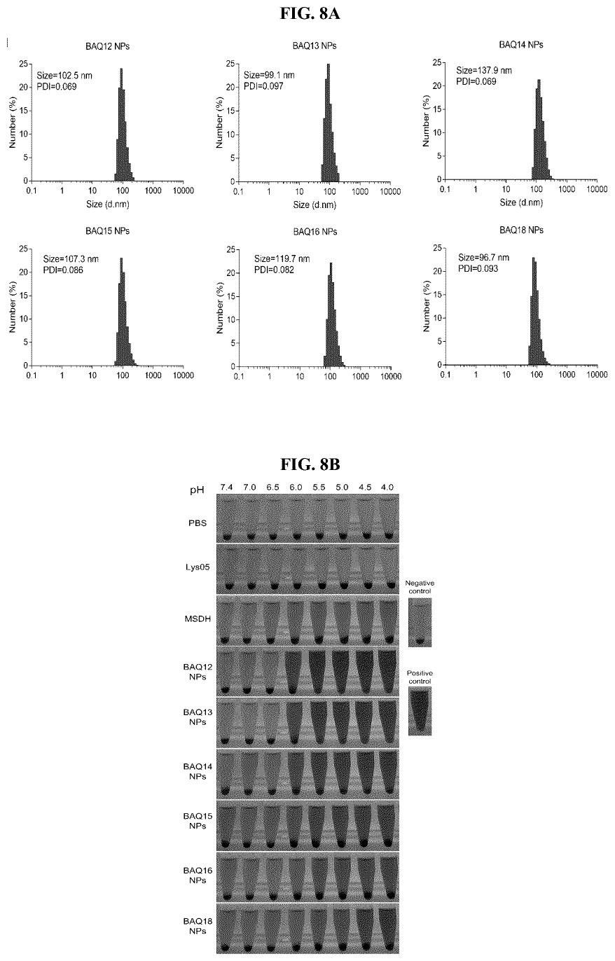

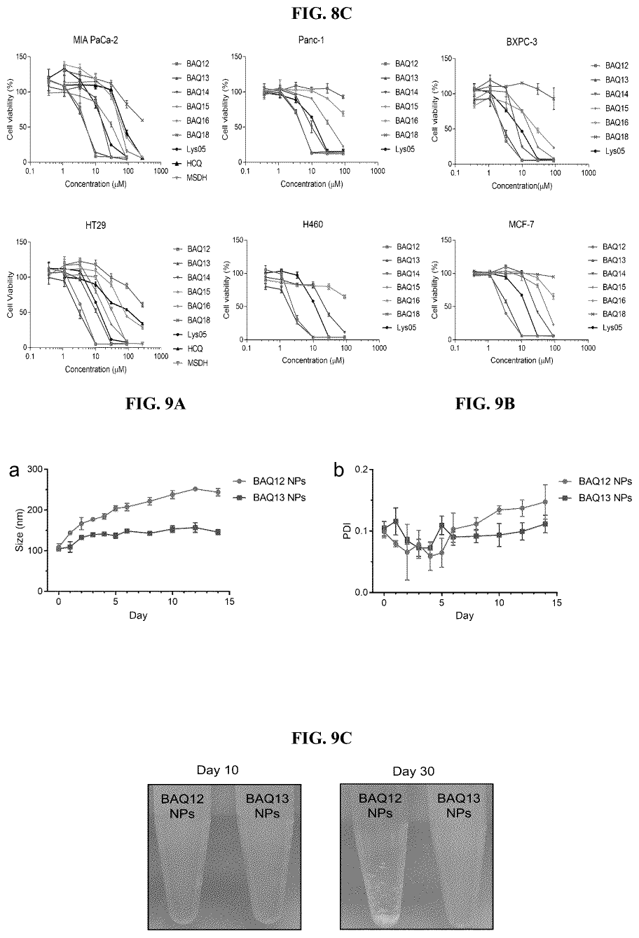

The compounds (BAQ12-BAQ18) were synthesized and structurally confirmed by ¹H NMR, ¹³C NMR and HRMS spectra (FIG. 7). In contrast to Lys05 and MSDH, BAQ12-BAQ18 were not completely soluble in water as free base or hydrochloride salt forms. However, the lipophilic cations allowed for spontaneous self-assembly in water via nanoprecipitation, which resulted in homogeneous opalescent NP solutions. The assembled NPs of BAQ12-BAQ18 had similar nanoscale characteristics, including their sizes (100-140 nm), polydispersity index (PDI) values less than 0.1, and positive surface charges (˜+40 mV) (Table 1 and FIG. 8A). The pH-responsive dissociation behaviour was then assessed by monitoring the size changes of the particles (FIG. 2A). All BAQ NPs were intact under near-neutral conditions and dissociated under only relatively acidic conditions. The critical dissociation pH was 5.5-6.0 for BAQ12-BAQ14 and 5.0-5.5 for BAQ15-BAQ18. When protonated in an acidic environment, the BAQ12-BAQ18 lipophilic cations turned into amphiphilic molecules and acquired surface activity. A haemolysis test was then utilized to evaluate the pH-responsive biomembrane disruption ability of the compounds. None of the NPs had haemolytic effects under approximately neutral conditions (pH≥6.5) but started to induce haemolysis when the pH was less than 6.0 (FIG. 2B and FIG. 8B). Among the compounds, BAQ12 NPs and BAQ13 NPs exhibited the strongest haemolytic activity, inducing up to 90% haemolysis under simulated lysosomal conditions (pH 4.0-5.5); in contrast, BAQ14 NPs induced moderate haemolysis (70%), and BAQ15-BAQ18 NPs only yielded ˜50% haemolysis. In the control groups, the conventional lysosomal detergent MSDH exhibited only a weak haemolytic response to pH, and Lys05 without detergence did not elicit observable haemolysis in the whole pH range at the same concentration. Because LMP is a potential stimulus for apoptosis, BAQ12 and BAQ13, the detergence of which can be activated in lysosomes, might be effective in inducing cancer cell death directly. Furthermore, upon titration with hydrochloride (HCl), BAQ12 NPs and BAQ13 NPs displayed obvious pH plateaus within a narrow pH range (at approximately pH 6.0), indicating their strong pH buffering capacity (FIG. 2C). In contrast, the pH values of the other NPs (BAQ14-BAQ18) decreased proportionally, and only short pH plateaus were observed for Lys05 (pH 7.2) and MSDH (pH 6.2). Since sufficient acidification is required for lysosomal degradation, BAQ12 and BAQ13, with their strong H⁺ buffering capacity, showed greater potential than the other compounds to induce lysosomal dysfunction and could therefore impair tumour cell growth.

TABLE 1
Nanoparticle characterization and IC50 values on cancer cells of BAQ derivatives.
(Data are mean values ± SD, n = 3)

| | | | Zeta | IC₅₀ (μM) in the 24 h treatment | | | | | |
|---|---|---|---|---|---|---|---|---|---|
| | | | Potential | MIA | | | | | |
| Compound | Size (nm) | PDI | (mV) | PaCa-2 | PANC-1 | BXPC-3 | HT29 | H460 | MCF-7 |
| BAQ12 | 102.5 ± 3.1 | 0.069 | 36.4 ± 1.5 | 4.1 ± 0.8 | 4.5 ± 0.9 | 2.9 ± 0.6 | 3.5 ± 1.2 | 2.7 ± 0.5 | 3.1 ± 0.1 |
| BAQ13 | 99.1 ± 2.1 | 0.097 | 39.7 ± 1.9 | 4.2 ± 1.0 | 4.3 ± 1.0 | 3.1 ± 0.2 | 3.0 ± 1.1 | 2.2 ± 0.4 | 3.7 ± 0.3 |
| BAQ14 | 137.9 ± 4.6 | 0.069 | 39.1 ± 0.4 | 16.0 ± 1.8 | 10.4 ± 1.8 | 7.5 ± 0.7 | 9.6 ± 2.0 | 24.0 ± 0.3 | 28.5 ± 3.5 |
| BAQ15 | 107.3 ± 2.6 | 0.086 | 38.0 ± 2.9 | 42.0 ± 11.7 | 34.5 ± 3.9 | 17.2 ± 2.0 | 16.1 ± 2.5 | 24.1 ± 0.4 | >60 |
| BAQ16 | 119.7 ± 2.1 | 0.082 | 40.4 ± 0.5 | 60.7 ± 13.9 | >90 | 27.6 ± 2.8 | >80 | >90 | >90 |
| BAQ18 | 96.7 ± 1.6 | 0.093 | 39.5 ± 0.8 | >90 | >90 | >90 | >100 | >90 | >90 |
| Lys05 | — | — | — | 16.7 ± 4.7 | 13.3 ± 2.0 | 7.8 ± 0.9 | 10.6 ± 3.4 | 11.1 ± 0.6 | 12.3 ± 0.7 |
| HCQ | — | — | — | 79.6 ± 8.5 | — | — | >75 | — | — |
| MSDH | — | — | — | 55.5 ± 7.1 | — | — | 30.3 ± 4.8 | — | — |

To verify the therapeutic effects of BAQ12-BAQ18, a preliminary screening was conducted using an MTS assay on various cancer cell lines. Within 24 h treatment, these derivatives exhibited anti-proliferative effects at different levels. BAQ12 and BAQ13 were highly effective and showed ˜3-fold, ˜20-fold and ˜10-fold higher potency than Lys05, HCQ and MSDH, respectively, but the activity decreased steadily as the hydrophobic tails extended from 14 to 18 carbons (Table 1 and FIG. 8C). This decrease was due to gradual declines in the detergence and H⁺ buffering capacity of compounds. Based on the results above, BAQ12 and BAQ13 were then selected as representatives for construction of BAQ ONNs in the following studies.

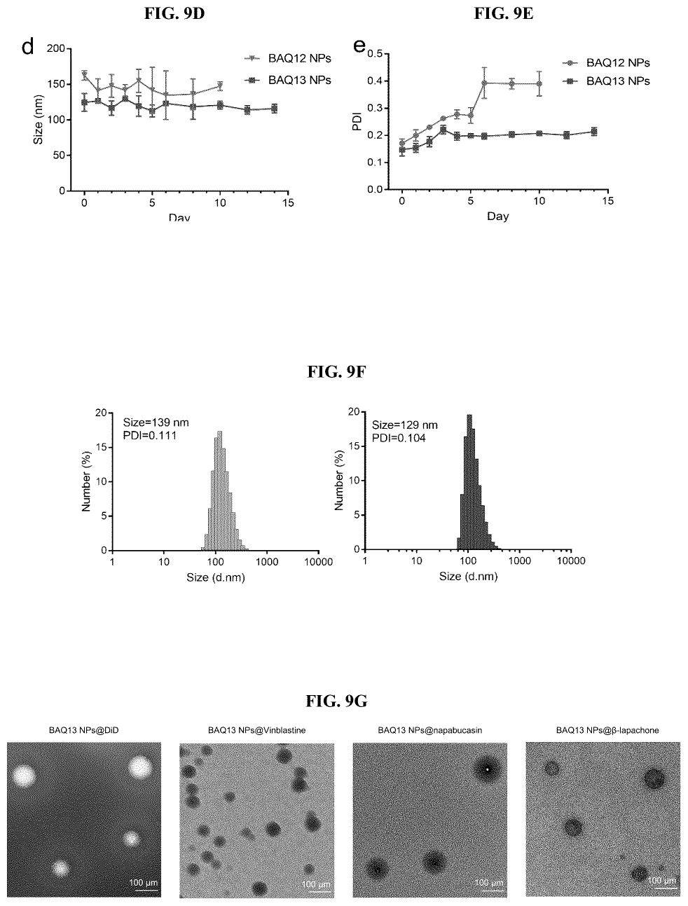

pH-responsive assembly and high drug-loading efficiency. The pH-responsive assembly dissociation phase transition of BAQ ONNs was then determined by transmission electron microscopy (TEM). At pH 7.4, the NPs exhibited a strong Tyndall effect and displayed liposome-like nanostructures with ˜100 nm diameters and bilayer thicknesses of ˜5 nm (FIG. 2D). These results were consistent with the dynamic light scattering (DLS) measurements. In contrast, at pH 5.0, the solution lost its Tyndall effect, and the vesicles were absent under TEM, which demonstrated that the NPs were dissociated under this condition (FIG. 2E). The release behaviour of BAQ ONNs at physiological pH (7.4) and lysosomal pH (5.0) was then investigated. As shown in FIG. 2F, BAQ12 NPs and BAQ13 NPs were released almost completely over 8 h (˜90%) at pH 5.0, but under the neutral condition, only ˜10% agents were released over 24 h. Considering that lysosomes maintain a pH in the range of 4.0-5.5, it's believed that BAQ ONNs will dissociate into free small molecules upon arrival in these compartments and will thus exert therapeutic effects. The critical aggregation concentrations (CACs) of BAQ12 NPs and BAQ13 NPs were measured to be 0.76 μM and 0.25 μM, respectively (FIG. 2G). The 3-fold difference observed between them indicated that BAQ13 could form NPs more easily than BAQ12 despite a difference of only one methylene unit between their molecular structures. The two NPs also exhibited decent stability in particle size over a relatively long duration at room temperature, even in the presence of 10% serum or 0.5 mM bovine serum albumin (FIGS. 9A-9F). In addition, BAQ13 NPs showed higher stability than BAQ12 NPs in such long-term storage, which is likely due to their different CACs.

Next investigation was whether liposome-like BAQ ONNs can encapsulate additional agents. Upon nanoprecipitation of BAQ13 and various agents, homogeneous NPs with monomodal size distributions spontaneously formed (Table 2 and FIG. 9G). BAQ13 NPs exhibited high drug-loading content (up to 50%, mass ratio) along with approximately 90% drug encapsulation efficiency (Table 2), which indicated that BAQ13 NPs could surpass the drug-loading limitations of the conventional liposome- and polymeric-based drug delivery systems. It is very encouraging that these simple NPs composed of single small-molecule therapeutic entities exhibit such a powerful drug-loading capacity.

TABLE 2
Parameters of drug loading using BAQ13 NPs.

| | NPs:drug | Drug- | | | | |
|---|---|---|---|---|---|---|
| Drugs or | (mass | loading | Encapsulation | Size/ | | |
| Dyes | ratio) | content | efficiency | diameter | | PDI |
| DiD (Dye) | 1:1 | 50% | 100% | 120 | nm | 0.1 |
| Bortezomib | 1:1 | 50% | 89% | 92 | nm | 0.09 |
| β-lapachone | 2:1 | 33% | 95% | 128 | nm | 0.047 |
| JQ1 | 1:1 | 50% | 92% | 110 | nm | 0.048 |
| Rapamycin | 1:1 | 50% | 90% | 85 | nm | 0.15 |
| Etoposide | 1:1 | 50% | 86% | 69 | nm | 0.104 |
| Apoptozole | 1:1 | 50% | 93% | 85 | nm | 0.099 |
| Vinblastine | 1:1 | 50% | 95% | 60 | nm | 0.133 |
| Lenalidomide | 1:1 | 50% | 88% | 85 | nm | 0.069 |
| Napabucasin | 4:1 | 20% | 85% | 110 | nm | 0.101 |

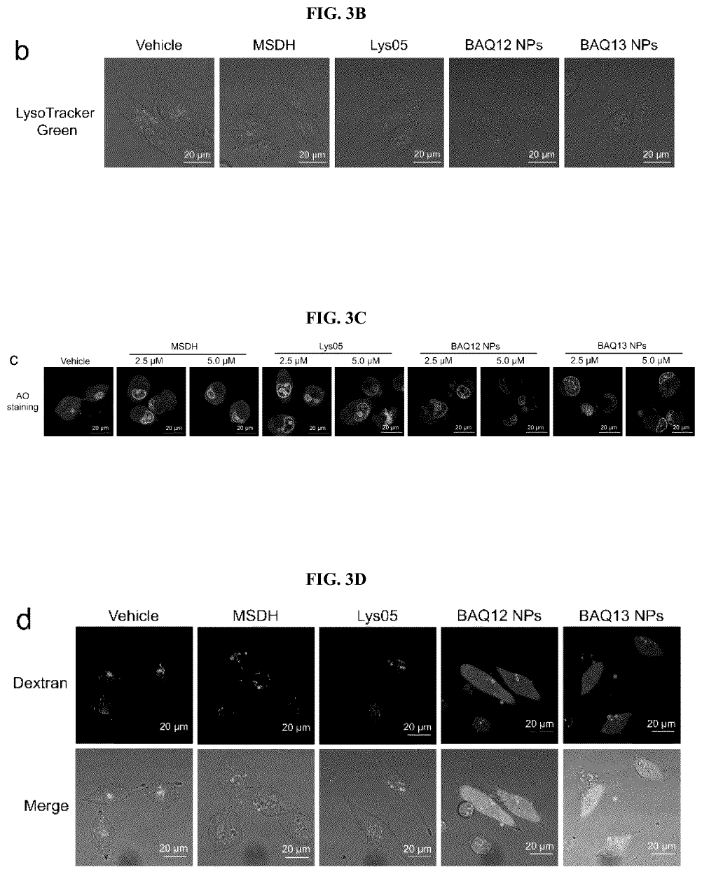

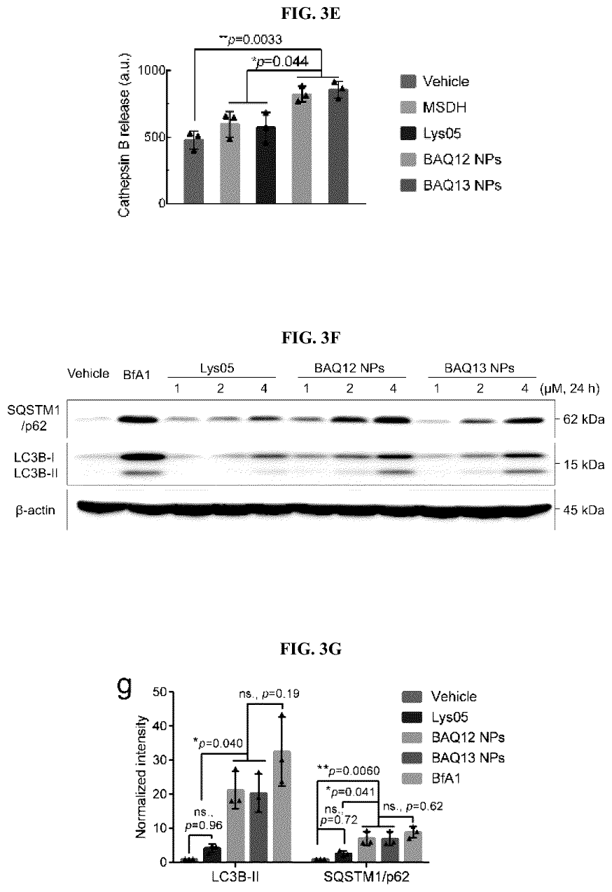

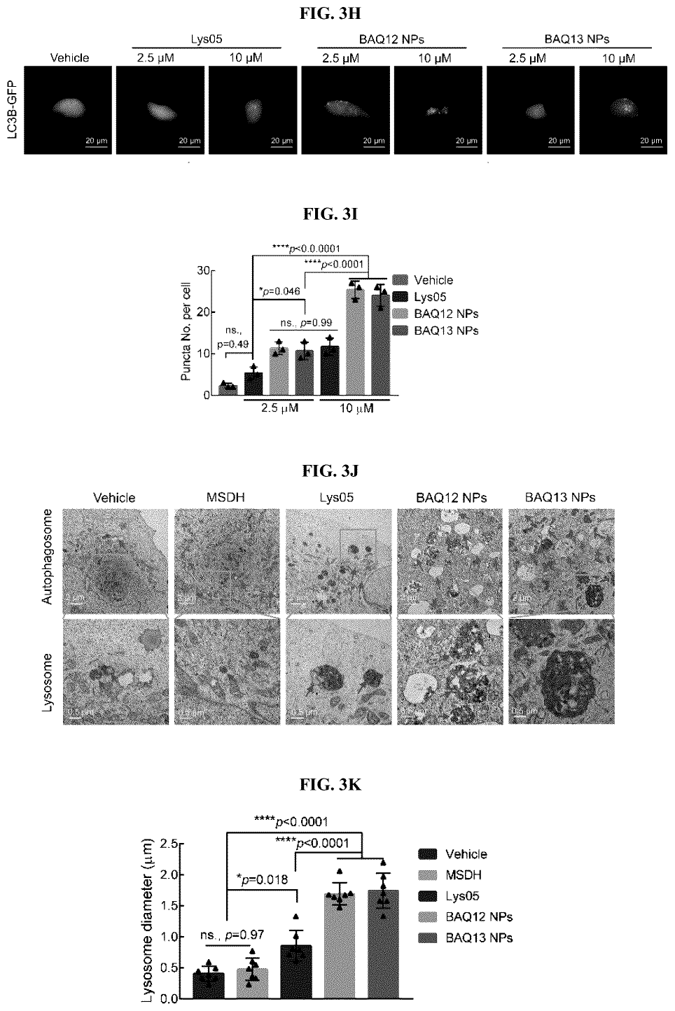

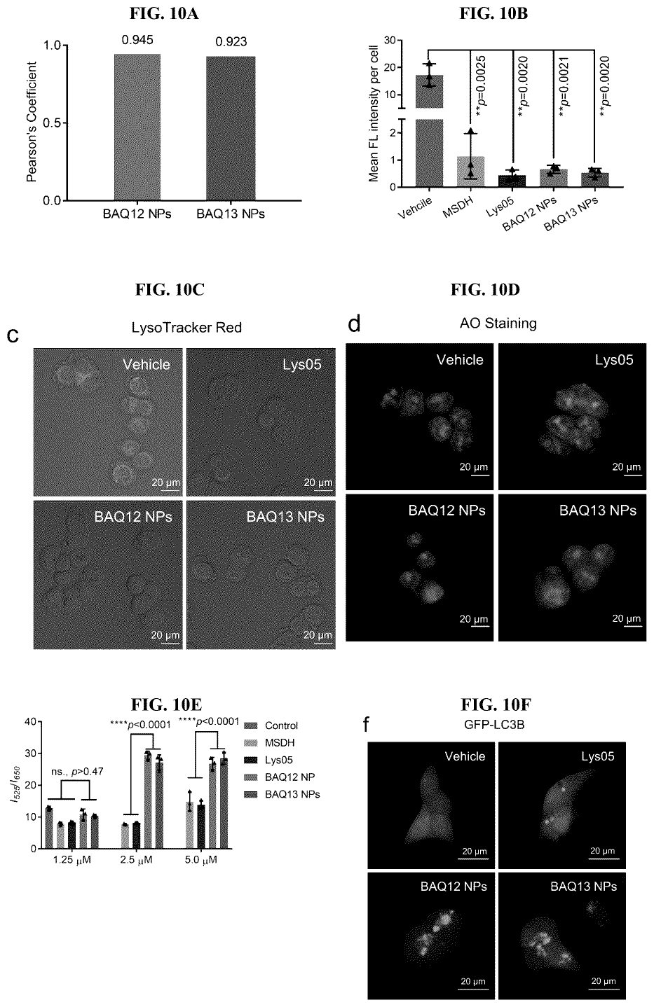

Accumulation in lysosomes and lysosomal disruption. To verify the lysosomal accumulation of BAQ ONNs, the near-infrared fluorescent dye, 1,1′-dioctadecyl-3,3,3′,3′-tetramethylindodi-carbocyanine (DiD) was loaded for labelling and tracking. As expected, the lysosome puncta (green) in MIA PaCa-2 cells stained by Dextran-Alexa Fluor 488 (AF488) overlapped consistently with the DiD-labelled NPs (red), suggesting that BAQ ONNs were quickly taken up by cells and accumulated in lysosomes (FIG. 3A and FIG. 10A). Upon this accumulation, BAQ ONNs reduced the LysoTracker-positive puncta, showing their ability to deacidify lysosomes similarly to Lys05 and MSDH (FIG. 3B and FIGS. 10B-10C).

The induction of LMP by BAQ12 and BAQ13 was investigated by live cell staining using the dye acridine orange (AO). Compared to those treated with Lys05 and MSDH, the cells treated with BAQ12 NP or BAQ13 NPs exhibited reduced numbers of red puncta and increased ratios of green to red fluorescence, suggesting that BAQ ONNs have an increased capability to induce lysosomal disruption in cancer cells. (FIG. 3C, and FIGS. 10D-10E). This LMP effect was further confirmed by detecting the release of Dextran-AF488 from lysosomes. As shown in FIG. 3D, treatment with BAQ ONNs resulted in a diffuse staining pattern throughout the cytoplasm, indicating lysosomal leakage, whereas the fluorescence in control cells appeared restricted to punctate structures, representing intact lysosomes. With their LMP function, BAQ ONNs were demonstrated to induce the release of cathepsin B from isolated lysosomes, which is an important trigger of apoptosis (FIG. 3E). As LMP was not observed in MSDH-treated cells, the results suggested that BAQ12 and BAQ13 represent the next generation of lysosomotropic detergents.

Autophagy inhibition. To explore the effect of BAQ ONNs on autophagy, the levels of microtubule-associated protein 1 light chain 3 (LC3) and Sequestosome 1 (SQSTM1)/p62 protein were measured, which are often used to monitor changes in the autophagy process. During autophagy, the cytosolic form of LC3 (LC3-I) is converted into the lipid modified form (LC3-II), which is then recruited to the autophagosomal membrane. Meanwhile, the autophagy substrate SQSTM1/p62 protein is degraded via selective incorporation into autophagosomes. Therefore, increased levels of both LC3-II and SQSTM1/p62 should be observed when autophagy is inhibited, while increased LC3-II levels and decreased SQSTM1/p62 levels should be observed if autophagy is activated. As shown in FIGS. 3F-3G, compared to the untreated cells and Lys05-treated cells, MIA PaCa-2 cells treated with BAQ ONNs showed significant concentration-dependent increases in both LC3B-II and SQSTM1/p62 protein levels. Such increases were also observed after treatment with bafilomycin A1 (BfA1), a known autophagy inhibitor. These findings indicate that BAQ ONNs can inhibit cellular autophagy more effectively than Lys05.

The autophagy-inhibiting effect was then confirmed by using LC3B-GFP imaging, as the formation of fluorescent LC3-II puncta in cells can be used to visualize the accumulation of autophagosomes. The cells treated with BAQ ONNs generated conspicuous LC3B-GFP puncta in a concentration-dependent manner (FIG. 3H and FIG. 10F). The LC3B-GFP puncta per cell were quantified, which revealed the higher autophagy inhibition potency of BAQ ONNs than Lys05 (FIG. 3I). For further verification, TEM was used to monitor cell micromorphological changes. As expected, compared to Lys05 and MSDH, BAQ ONNs induced the formation of larger autophagic vesicles (AVs) or autophagosomes in cells, which further confirmed the improved autophagic inhibition effects of BAQ ONNs (FIGS. 3J-3K). Taken together, the findings indicated that BAQ12 NPs and BAQ13 NPs surpassed the parental Lys05 in inhibiting autophagy; thus, BAQ ONNs represent a generation of nanoformulated autophagy inhibitors.

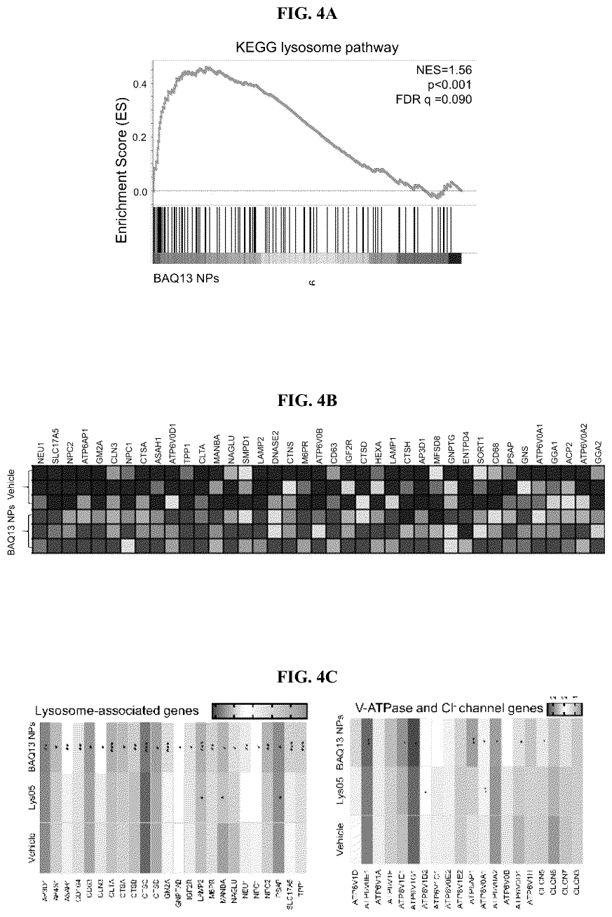

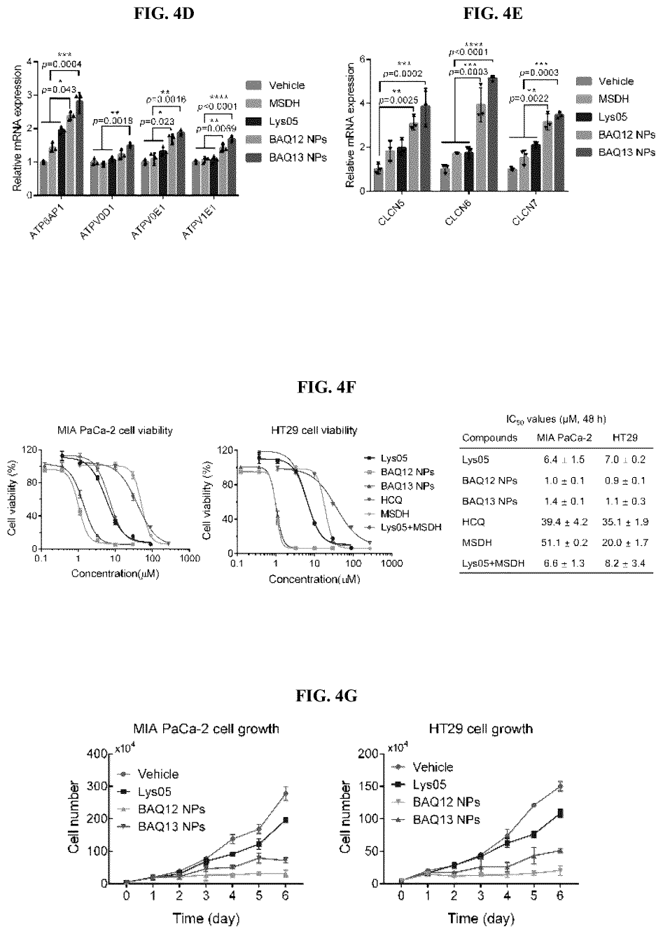

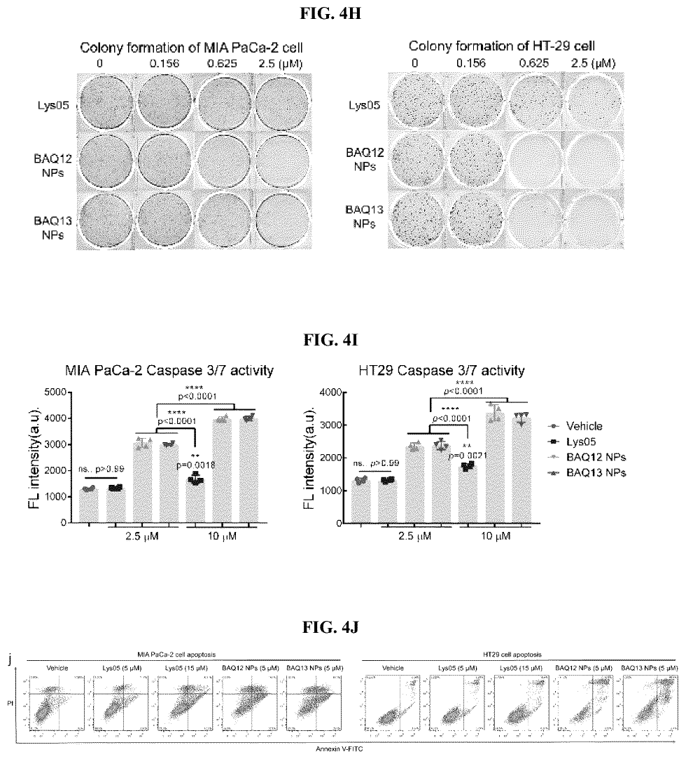

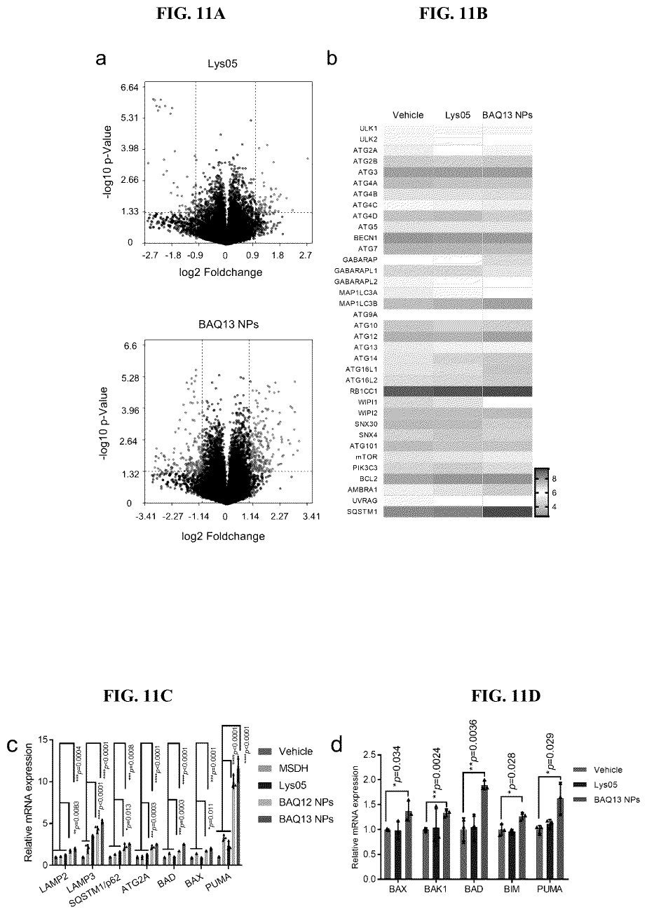

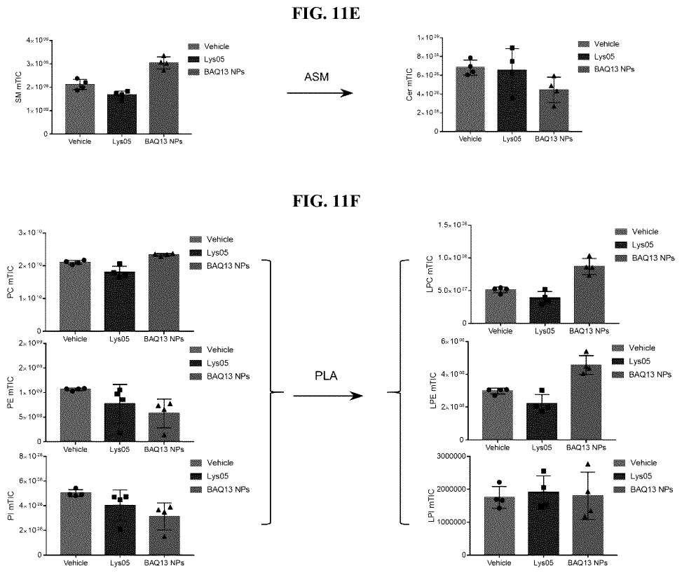

Proton-sponging effect and lysosomal dysfunction. As cationic molecules, both BAQ12 and BAQ13 possess strong H⁺ buffering capacity, an essential characteristic of materials with proton-sponging effects (FIG. 2C). The TEM results above indicated that the BAQ ONNs could significantly enlarge lysosomes (FIGS. 3J-3K), demonstrating the proton-sponging effects of BAQ ONNs. To further investigate these effects, the transcriptomic changes in MIA PaCa-2 cells post-treatment using RNA sequencing (RNA-seq) was characterized. A total of 13234 genes were tested, and their expression levels were compared among vehicle, Lys05 and BAQ13 groups. Using volcano plot analysis, 165 differentially expressed genes (DEGs) (fold change≥2 andp value≤0.05) was found in the Lys05 group compared with the vehicle group, including 62 upregulated genes and 103 downregulated genes. In comparison, 390 DEGs were found in the BAQ13-treated cells, including 209 upregulated genes and 181 downregulated genes (FIG. 11A). Enrichment analysis of the gene set with the Kyoto Encyclopedia of Genes and Genomes (KEGG) revealed that BAQ13 NPs induced robust upregulation of lysosome-associated genes, such as V-ATPase, Cl-channel, protease, and lysosome-associated membrane protein (LAMP) genes (FIGS. 4A-4C and FIG. 11B). qPCR analysis also confirmed the upregulation of V-ATPase and Cl-channel genes, which remarkably indicated that the BAQ ONNs had strong proton-sponging properties (FIGS. 4D-4E).

The upregulation of important lysosomal enzyme genes, such as cathepsin and NEU1, also emphasized on the lysosomal dysfunction caused by BAQ ONNs (FIGS. 4B-4C). LAMP genes that are thought to be partly responsible for maintaining lysosomal integrity were upregulated as well, which indicated the function of BAQ ONNs in lysosomal disruption (FIG. 11C). The BAQ ONN treatment groups exhibited high transcriptomic levels of proapoptotic genes (BAX, BAK1, BAD, BIM and PUMA), revealing the enhanced proapoptotic effects (FIGS. 11C-11D). The BAQ ONN-induced lysosomal dysfunction was then confirmed using lipidomic analysis. BAQ13 NPs induced accumulation of the acid sphingomyelinase (ASM) precursor sphingomyelin (SM) and led to decreases in the levels of its product, ceramide (Cer) (FIG. 11E). In addition, the levels of phospholipase A (PLA) precursors, including phosphatidylcholine (PC), phosphatidylethanolamine (PE) and phosphatidylserine (PI), were decreased and the levels of their corresponding products, lysophosphatidylcholine (LPC), lysophosphatidylethanolamine (LPE) and lysophosphatidylserine (LPI), were increased (FIG. 11F).

Nanocarriers of the present invention can also include conjugates wherein R¹ is pheophorbide-a to form a pheophorbide-a bisaminoquinoline conjugate (PBC). PBC nanoparticles are a lysosome-targeted morphologically transformable nanoassembly. PBC nanoparticles have a liposome-like morphology in physiological conditions and could transform into nanofibers after accumulation in lysosomes. The formed nanofiber in lysosomes can cause lysosomal dysfunction and trigger apoptosis of cancer cells. Since containing the photosensitization group in the structure, this nanoparticle also supports a highly effective lysosome-based photodynamic treatment that can intrinsically overcome the autophagy-associated drug resistance.

### Example 3: In Vitro Studies

Cell line and cell culture. The human pancreatic cancer cell lines (MIA PaCa-2, BXPC3, and PANC-1) were originally purchased from ATCC and were kindly provided by Dr. Shiro Urayama's Lab. HT29, HCT116, H460, MCF7, NIH/3T3, and IMR-90 cell lines were purchased from ATCC. Bone marrow cells were collected from the leg bone marrows of FVB/N mice. All the cells were cultured at 37° C. in a humidified atmosphere of 5% CO₂/95% air using the corresponding medium supplemented with 10% fetal bovine serum, 100 μg mL⁻¹ penicillin and 100 units mL⁻¹ of streptomycin according to ATCC protocol. All the cell lines have been tested for mycoplasma contamination routinely.

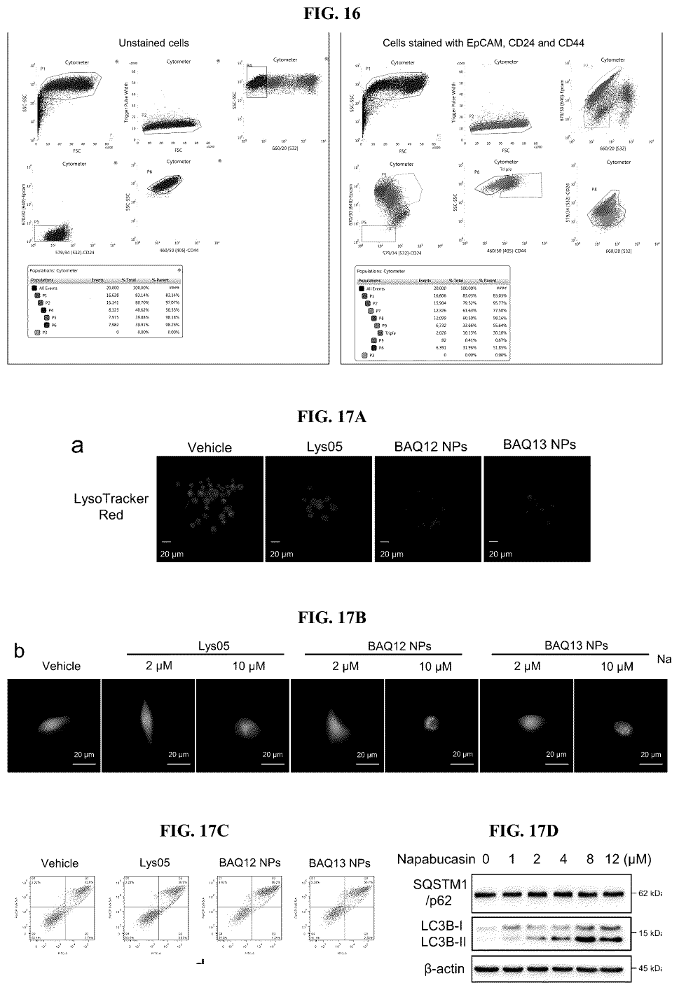

Establishment of patient-derived pancreatic cancer stem cells (PCSCs). The pancreatic patient tissue was donated by Dr. Shiro Urayama's Lab from UC Davis Medical Center. Patient consent was obtained for the use of “Remnant Clinical Biospecimens” in accordance with the Institutional Review Board (UC Davis IRB Protocol #244896). The patient tumour tissue was harvested using Collagenase IV and Dispase (Stem Cell Technologies, Vancouver, Canada) and strained through 70 μm filters. By labeled with the following antibodies (Biolegend, CA, USA): anti-CD44 (IM7, Cat: #338823), anti-CD326 (9C4, Cat: #324207) and anti-CD24 (ML5, Cat: #311119), the cells were isolated using a BD FACSAria II Cell Sorter (FIG. 16). Purified PCSCs were collected and maintained in Essential 8 Flex Medium (Thermo Fischer) and trypsinized using Gentle Cell Dissociation Reagent (Stem Cell Technologies, Vancouver, Canada). In tumour sphere-formation assay, the cold single cell suspension of PCSCs in Essential 8 Flex Medium was mixed with cold Matrigel (1:1, volume ratio), followed by slowly and uniformly dropping them (100 μL) into well center on a 24-well plate (5,000 cells per well). The Matrigel was allowed to solidify in a humidified incubator at 37° C. for 45-60 min, and the warm media (500 μL) was added into each well. The tumour sphere was allowed to be formed in two weeks. For tumourigenicity in vivo, PCSCs were counted and resuspended into a mixture of PBS and Matrigel (1:1) and subsequently injected subcutaneously into the flanks of NRG mice.

Cell viability, cell growth and colony formation. Cell viability was assessed by the MTS assay. Briefly, cells in 96-well plates (4,000 cell per well) were treated as indicated, followed by the incubation with MTS regents for 4 h. OD values (490 nm) were determined via a microplate reader. Results were shown as the average cell viability calculated from the formula of [(ODₜᵣₑₐₜ−OD_{blank})/(OD_{control}−OD_{blank})×100%]. Drug combination data were analyzed by Combenefit 2.02. In cell growth assay, cells in 6-well plates (50,000 cell per well) were treated as indicated and were counted manually every 24 h. Colony formation assay was also performed on 6-well plates with a starting density of 1,000-2,000 cells per well. After incubated as indicated for 10-20 days, cells were washed with PBS and stained with the solution of crystal violet and methanol for 20 min.

Apoptosis and caspase-3/7 activity. Cell apoptosis was measured using FITC-Annexin V/PI Apoptosis kit (AnaSpec). Briefly, the treated cells were stained according to the manufacturer's instructions and were detected on a BD FACSCanto II flow cytometer. Data were analyzed by FlowJo 7.6.1. In caspase 3/7 activity assay, cells in 96-well plates (10,000 cells per well) were treated as indicated, followed by adding AMC caspase-3/7 assay kit (50 μL per well, AnaSpec). The fluorescence intensity (λₑₓ=356 nm, λₑₘ=442 nm) was recorded by a microplate reader.

Cell uptake and deacidification. For cell uptake, lysosomes were labeled with Alexa Fluor 488-dextran (10 kDa, 100 μg mL⁻¹, Thermo Fisher) for 36 h, followed by incubation with DiD-loaded BAQ NPs (10 uM, 1:10, mass ratio) for 2 h. In lysosomal deacidification analysis, cells were treated for 2 h and incubated with LysoTracker Red (100 nM, Thermo Fisher) for 1 h. Cell images were obtained using a Zeiss Confocal Microscope and analyzed by Zen 2.3 and ImageJ 1.51s.

Lysosome integrity. Lysosomal integrity was measured in living cells by using the AO (Thermo Fisher) or Alexa Fluor 488-dextran (10 kDa) staining. For AO staining, the treated cells were incubated with AO (2 μg mL⁻¹) for 1 h. For dextran staining, the dextran-loaded cells were exposed to treatments for 12 h. Images were captured under a Zeiss Confocal Microscope and analyzed by Zen 2.3 and ImageJ 1.51s.

LC3B-GFP imaging. Cells in a 96-well plate (5,000 cell per well) were transfected by the autophagy sensor LC3B-GFP (Thermo Fisher) for 12 h. After treated as indicated for 4 h, cells were visualized by a fluorescence microscope (Olympus). The puncta per well were quantified using ImageJ 1.51s.

Lysosome isolation and cathepsin release. Lysosomes were isolated using a Lysosome Enrichment Kit (Thermo Fisher) according to the manufacturer's protocol. The equal portions of isolated lysosomes were incubated as indicated for 12 h at 37° C. and then was centrifuged at 15,000×g for 30 min at 4° C. to pellet intact lysosomes. The release of cathepsin B into the supernatant was determined (Ex=380 mm, Em=460 mm) after a 2 h incubation with 200 μM fluorogenic Cathepsin B Substrate III (Z-Arg-Arg-AMC).

Haemolysis. Red blood cells (2%) in PBS (10 mM, pH7.4) were incubated with NPs for 4 h at 37° C. After centrifugation at 500×g for 5 min, the extent of haemolysis was spectrophotometrically determined according to the amount of haemoglobin in supernatants (540 nm). The haemolysis assay was used to assess the pH-dependent detergence ability and toxicity of NPs.

Western Blot. The cell or tumour samples were lysed with RIPA Buffer (Thermo Fisher). After centrifugation at 4° C. (15 min, 12,000×g), the concentrations of proteins in supernatant and determined by Bradford Protein Assay dye (Bio-Rad). Immunoblotting was performed routinely and were developed using a ChemiDoc™ MP imaging system.

TEM of cells and tumour tissue. MIA PaCa-2 cells in 8-well slide plates (30,000 cell per well, Lab-Tek) were treated as indicated for 48 h. The freshly harvested tumours were cut into 1 mm³ pieces. Samples were fixed with the 0.1 M cacodylate buffer containing 2.5% glutaraldehyde plus 2% paraformaldehyde, and transferred onto carbon square mesh, followed by observation under Talos L120C TEM.

RNA-seq. Total RNA was extracted by the RNeasy Mini Kit (Qiagen, Germany) from the treated MIA PaCa-2 cells (5 μM, 24 h). Samples were submitted to the UC Davis Comprehensive Cancer Center's Genomics Shared Resource (GSR) for RNA-Seq analysis. Stranded RNA-Seq libraries were prepared from 100 ng total RNA using the NEBNext Ultra Directional RNA Library Prep Kit (New England BioLabs). Subsequently, libraries were combined for multiplex sequencing on an Illumina HiSeq 4000 System (2×150 bp, paired-end, >20×10⁶ reads per sample). The data of normalized genes read counts were analyzed using fold change and t test. The Differentially expressed genes (DEGs) were collected for the signaling pathways enrichment by Funrich software 3.1.3. The gene sets were from MSigDB database (Broad Institute). GSEA was performed using GSEA version 3.0 in KEGG gene sets category online, with the following parameters: n=1,000 permutations, where p-adjust <0.05, and FDR <0.05 were considered significant.

qPCR. The total RNA was isolated using the TRIZOL reagent (Invitrogen) and the phenol-chloroform extraction method. The cDNA was synthesized using SuperScript II reverse transcriptase (Invitrogen) with 2 μg of total RNA in a 20 μL reaction. The resulting cDNA was diluted 1:20 in nuclease-free water and 4 μL was used per qPCR reaction with triplicates. qPCR was carried out using Power SYBR Green PCR Master Mix (Thermo Fisher) on a CFX96 Real-Time PCR Detection System (Bio-Rad) including a non-template negative control. Amplification of GAPDH was used to normalize the level of mRNA expression. The primer sequences were listed in Table 3.

TABLE 3
Primers and sequences used in RT-PCR analysis.

| Genes | Primers |
|---|---|
| BAD | 5′-CCCAGAGTTTGAGCCGAGTG-3′ (Forward) |
| | 5′-CCCATCCCTTCGTCGTCCT-3′ (Reverse) |
| BAX | 5′-CCCGAGAGGTCTTTTTCCGAG-3′ (Forward) |
| | 5′-CCAGCCCATGATGGTTCTGAT-3′ (Reverse) |
| BAK1 | 5′-GAGAGCCTGCCCTGCCCTCT-3′ (Forward) |
| | 5′-CCACCCAGCCACCCCTCTGT-3′ (Reverse) |
| BIM | 5′-GGCAAAGCAACCTTCTGATG-3′ (Forward) |
| | 5′-TAACCATTCGTGGGTGGTCT-3′ (Reverse) |
| PUMA | 5′-GACCTCAACGCACAGTACGAG-3′ (Forward) |
| | 5′-AGGAGTCCCATGATGAGATTGT-3′ |
| | (Reverse) |
| ATP6AP1 | 5′-CAGCGACTTGCAGCTCTCTAC-3′ (Forward) |
| | 5′-TGAAATCCTCAATGCTCAGCTTG-3′ |
| | (Reverse) |
| ATP6V0D1 | 5′-TTCCCGGAGCTTTACTTTAACG-3′ |
| | (Forward) |
| | 5′-CAAGTCCTCTAGCGTCTCGC-3′ (Reverse) |
| ATP6V1E1 | 5′-AACATAGAGAAAGGTCGGCTTG-3′ |
| | (Forward) |
| | 5′-GACTTTGAGTCTCGCTTGATTCA-3′ |
| | (Reverse) |
| ATP6V0E1 | 5′-GTCCTAACCGGGGAGTTATCA-3′ (Forward) |
| | 5′-AAAGAGAGGGTTGAGTTGGGC-3′ (Reverse) |
| LAMP2 | 5′-GAAAATGCCACTTGCCTTTATGC-3′ |
| | (Forward) |
| | 5′-AGGAAAAGCCAGGTCCGAAC-3′ (Reverse) |
| LAMP3/CD63 | 5′-CAGTGGTCATCATCGCAGTG-3′ (Forward) |
| | 5′-ATCGAAGCAGTGTGGTTGTTT (Reverse) |
| SQSTM1/p62 | 5′-GACTACGACTTGTGTAGCGTC-3′ (Forward) |
| | 5′-AGTGTCCGTGTTTCACCTTCC-3′ (Reverse) |
| ATG2A | 5′-TGTCCCTGTAGCCATGTTCG-3′ (Forward) |
| | 5′-TCAGGATCTCCGTGTACTCAG-3′ (Reverse) |
| CLCN5 | 5′-ATAGGCACCGAGAGATTACCAA-3′ |
| | (Forward) |
| | 5′-CTAACGAACCTGATAAAAGCCCA-3′ |
| | (Reverse) |
| CLCN6 | 5′-TCTCCTTACGGAAGATCCAGTT-3′ |
| | (Forward) |
| | 5′-AAGGTGGCAGACATGGAACAA-3′ |
| | (Reverse) |
| CLCN7 | 5′-CCACGTTCACCCTGAATTTTGT-3′ |
| | (Forward) |
| | 5′-AAACCTTCCGAAGTTGATGAGG-3′ |
| | (Reverse) |
| GAPDH | 5′-TGTGGGCATCAATGGATTTGG-3′ |
| | (Forward) |
| | 5′-ACACCATGTATTCCGGGTCAAT-3′ |
| | (Reverse) |

Lipidomics. MIA PaCa-2 cells were treated with compounds (2.5 μM) for 48 h, and 1.5 million cells in each group were collected to prepare the samples routinely for RPLC-QTOF analysis. The samples were run on a Vanquish UHPLC System, followed by data acquisition using a Q-Exactive HF Hybrid Quadrupole-Orbitrap Mass Spectrometer. The LC-MS data were processed using MS-DIAL 3.70. Statistical analysis was done by first normalizing data using the sum of the knowns, or mTIC normalization, to scale each sample. Normalized peak heights were then submitted to R 3.5.1 for statistical analysis. ANOVA analysis was performed with FDR correction and post hoc testing.

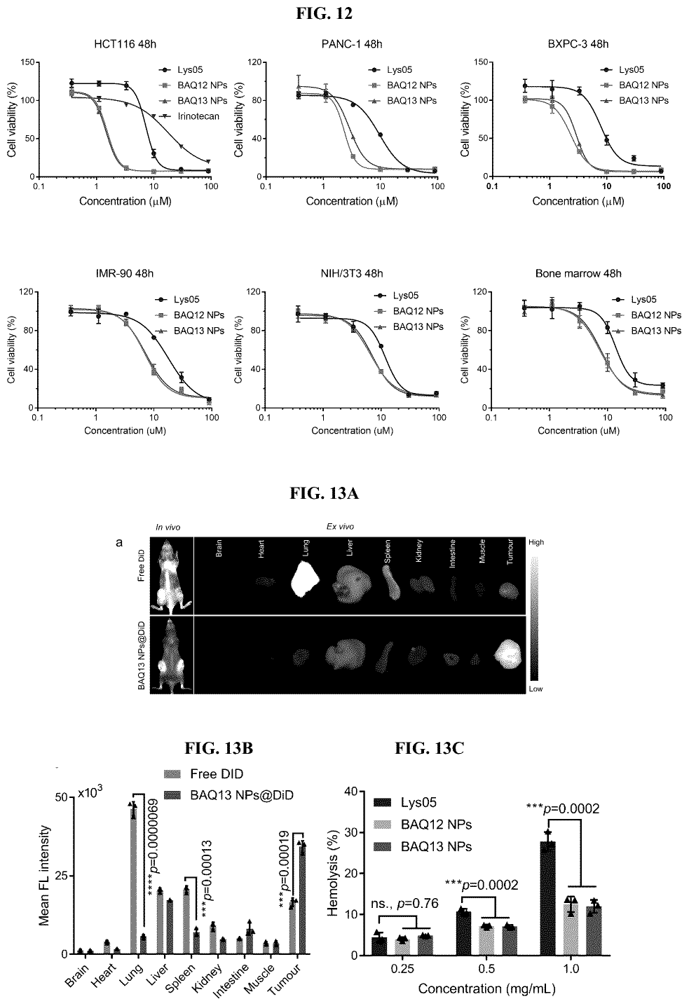

In vitro antitumour activity of BAQ derivatives. To systematically investigate the antitumour effects in vitro, three pancreatic cancer cell lines (MIA PaCa-2, BxPC-3, and PANC-1) and two colon cancer cell lines (HT29 and HCT116) were selected for a 48 h MTS assay. The BAQ ONNs showed IC₅₀ values of 1-3 μM and were thus approximately 5-fold, 30-fold and 20-fold more potent than Lys05, HCQ and MSDH, respectively (FIG. 4F, FIG. 12, and Table 4). These results also indicated that treatment with BAQ12 or BAQ13 alone was more effective than combination treatment with Lys05 and MSDH, suggesting that pronounced pharmacodynamic synergism occurred upon pharmacophore fusion. The results of cell growth and colony formation assays further demonstrated the inhibitory effects of BAQ ONNs on tumour cells (FIGS. 4G-4H). The improved anticancer activity of BAQ ONNs is attributable to their multiple functions in inducing LMP, lysosomal dysfunction and autophagy inhibition in cancer cells; these effects are considered to be important triggers of apoptosis. To examine the proapoptotic effects of BAQ ONNs, apoptosis signals in MIA PaCa-2 and HT29 cells was subsequently detected. BAQ ONN treatment resulted in significant elevations in both caspase 3/7 activity and apoptosis levels (FIG. 4I-4J). Lys05, the control, increased apoptotic signals in a concentration-dependent manner, but its effect at a high concentration close to the IC₅₀ was still milder than those of the low concentrations of BAQ ONNs. These results demonstrate that cancer cells are more likely to undergo apoptosis when treated with multifunctional BAQ entities than when treated with Lys05, whose main function is autophagy inhibition. In addition, compared to the panel of cancer cell lines, the non-cancerous cell lines including IMR-90 cells, NIH/3T3 cells, and bone marrow cells, showed relative insensitivity to BAQ ONNs, thus indicating the relative high safety of those compounds (FIG. 12 and Table 4).

TABLE 4
The calculated IC50 values. Data are presented as mean values ± SD.

| | IC₅₀ values from the 48 h MTS assay (μM) | | | | | |
|---|---|---|---|---|---|---|
| Drugs or | | | | | | Bone |
| nanoparticles | HCT116 | PANC-1 | BXPC-3 | IMR-90 | NIH/3T3 | marrow |
| Lys05 | 8.0 ± 0.7 | 10.8 ± 0.9 | 11.1 ± 1.7 | 15.5 ± 2.5 | 12.6 ± 0.9 | 14.0 ± 1.1 |
| BAQ12 NPs | 1.6 ± 0.1 | 1.9 ± 0.4 | 2.6 ± 0.3 | 6.23 ± 0.8 | 6.5 ± 0.7 | 7.8 ± 0.8 |
| BAQ13 NPs | 1.6 ± 0.1 | 2.6 ± 0.3 | 3.1 ± 0.1 | 6.7 ± 0.5 | 7.0 ± 0.5 | 8.0 ± 0.8 |
| Irinotecan | 17.0 ± 1.0 | — | — | — | — | — |

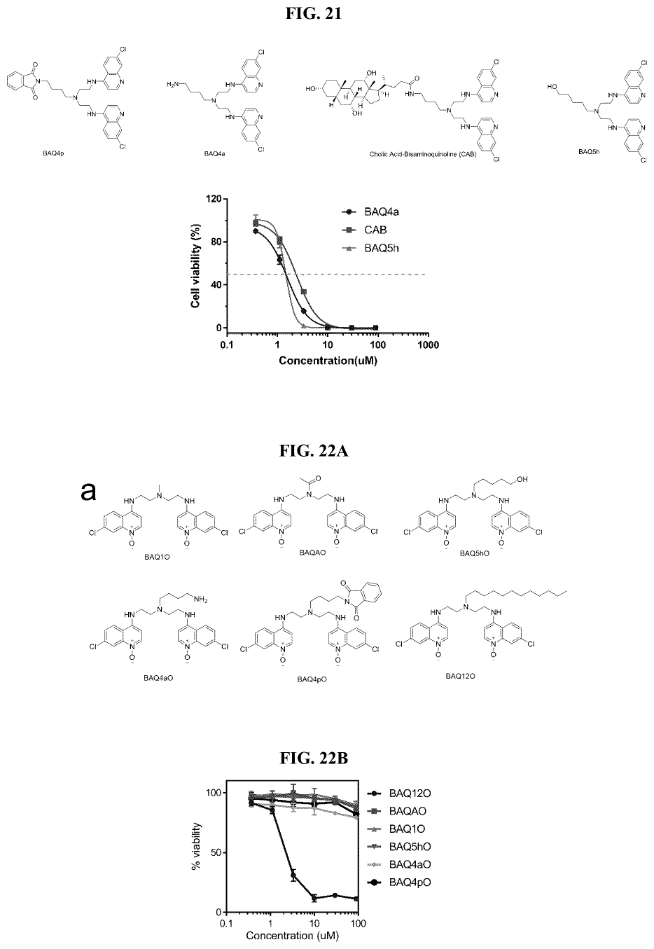

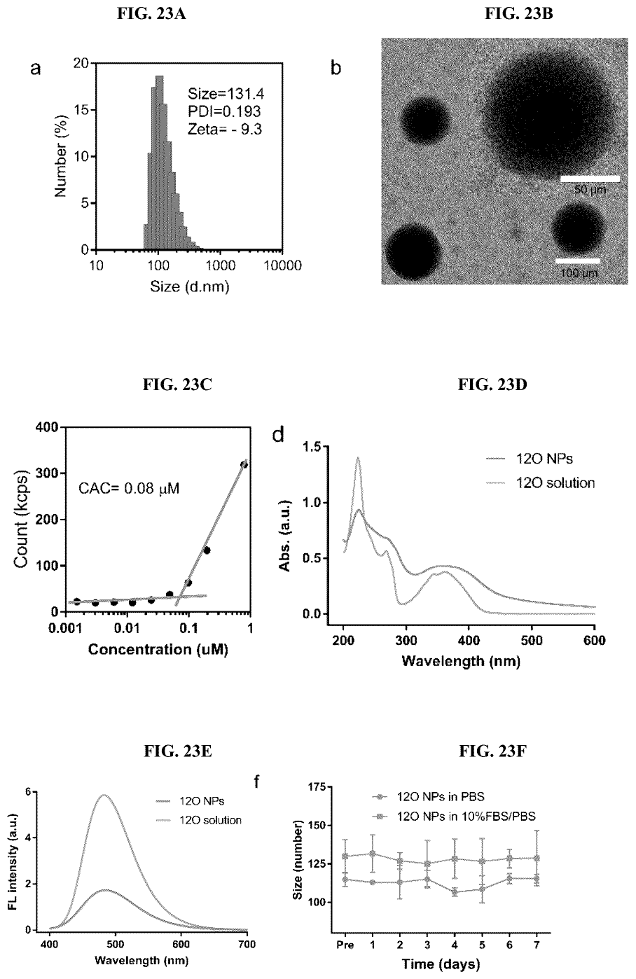

In vitro antitumour activity of BAQO derivatives. In vitro antitumor effects of BAQO derivatives were performed in pancreatic cancer stem cells (PCSCs). BAQ12O ONNs showed IC₅₀ values of less than 5 μM (FIG. 22B), whereas the other BAQO derivatives have showed no toxicity up to a concentration of 100 μM. These results show that BAQO derivatives can be versatile as a therapeutic agent or a drug delivery agent without contributing to cytotoxicity.

### Example 4: In Vivo Studies

Animal model. To establish the subcutaneous xenograft models, 5×10⁶ of MIA PaCa-2 cells, 2×10⁶ of HT29 cells or 2×10⁴ PCSCs suspended with Matrigel (Corning) and PBS mixture (1:1, volume ratio) were injected subcutaneously into the right flank of nude mice or NRG mice, respectively.

Animal feeding. All animal experiments were conducted in accordance with the protocol (#20265) approved by the Institutional Animal Care and Use Committee at the University of California, Davis. Female mice (4-6 week) including BALB/c nude mice (Envigo), NRG mice (Jackson Laboratory), and FVB/N mice (Charles River) were purchased and group-housed under standard conditions (22±1° C., humidity 50-60%, 12 h light/12 h dark cycle, free access to food and water).

In vivo treatment schedule. The NRG mice bearing MIA PaCa-2 xenograft tumours (˜100 mm³) were randomized into 5 groups (n=6), and then were subjected to iv injection every three days as indicated. For HT29 xenograft model, six groups of nude mice (n=6) with 100 mm³ of tumours were administrated every three days with vehicle (saline, iv), Lys05 (ip), BAQ12 NPs (iv), BAQ13 NPs (iv), Irinotecan (ip), respectively. The treatment on HT29 model was stopped on Day 24, and then the mice survival in each group was recorded, in which the mouse with a tumour larger than 1,000 mm³ was considered dead. For the co-delivery study, NRG mice (n=5) bearing PCSC tumours were treated with vehicle (saline, iv), napabucasin (ip), BAQ13 NPs (iv), BAQ13 NPs+napabucasin (iv and ip, respectively) and BAQ13 NPs@napabucasin (iv) every three days. The tumour volume and body weight were recorded before drug administration every time. At the end of the treatment, mice were sacrificed and the tumours were collected for further analysis.

In vivo toxicity studies. The toxicity of BAQ NPs was investigated on female FVB/N mice via iv injection. Mice were administrated with various concentrations (10 mg kg⁻¹, 20 mg kg⁻¹ or 40 mg kg⁻¹) of Lys05, Liposomes@Lys05, BAQ12 NPs, and BAQ13 NPs every two days. The status of mice was monitored every day and their body weight was recorded every two days. Blood samples were collected and sent to the UCD Comparative Pathology Laboratory for tests of complete blood count (CBC) and serum chemistry.

In vivo pharmacokinetic study. The jugular vein of female Sprague-Dawley rats (200-250 g) was implanted with a catheter for drug injection and blood collection (Harland, Indianapolis, IN, USA). Rats (n=3) were injected with free DiD, BAQ12 NPs@DiD (10:1, mass ratio) and BAQ13 NPs@DiD (10:1, mass ratio), respectively, which contained an equivalent dose of DiD (0.5 mg kg⁻¹). Blood samples were collected at the indicated time points and then were centrifuged to obtain the plasma. The plasma was diluted with DMSO (1:100), and the fluorescence intensity (λ_{Ex}=595 nm, λ_{Em}=665 nm) was measured by a microplate reader (SpectraMax M2).

In/ex vivo biodistribution. Nude mice bearing the HT29 tumours were subjected to iv administration of BAQ13 NPs@DiD (10:1, mass ratio) at a dose of 1.0 mg kg⁻¹ DiD. In vivo imaging studies were performed at the corresponding time point. Organs (brain, heart, lung, liver, spleen, kidney, intestines, and muscle) and tumours were collected from mice for ex vivo imaging. Biodistribution of BAQ13 NPs@ NAPA+DiD (10/2.5/1.0 mg kg⁻¹, iv) was studied on NRG mice bearing PCSC tumours. Both in vivo and ex vivo imaging studies were performed as above.

Statistics. Statistical analysis was performed using GraphPad Prism 7.0. Data are presented as mean values±SD, n=biological replicates or independent nanoparticle sample replicates. One-way ANOVA with the Tukey's multiple comparison test or two-tailed Student's t-test was used to calculate the p value as noted in each figure legend. ns., not significant; *p<0.05; **p<0.01; ***p<0.001; ****p<0.0001.

Data availability. The RNA-seq data have been deposited in the Gene Expression Omnibus (GEO) database under the accession code GSE154323 [https://www.ncbi.nlm.nih.gov/geo/query/acc.cgi?acc=GSE154323].

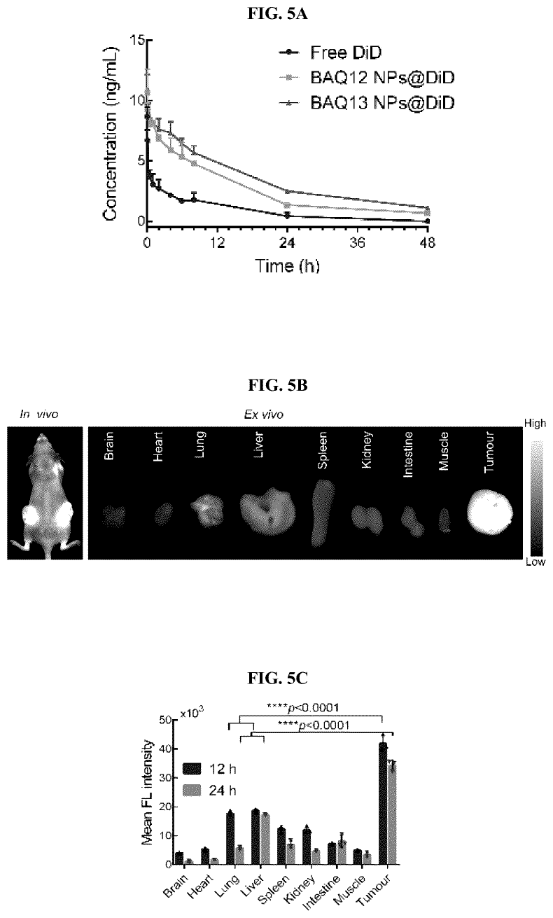

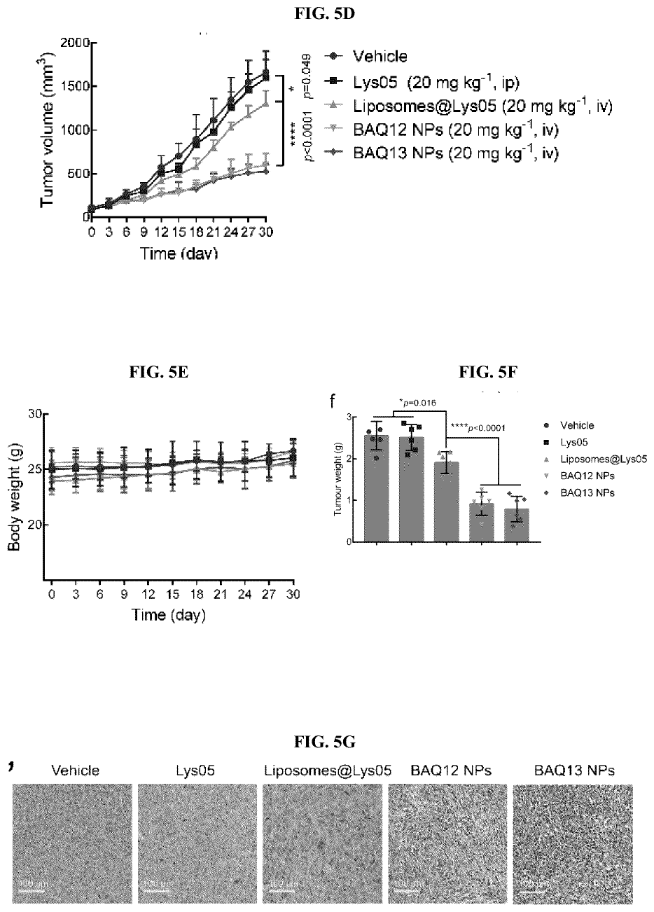

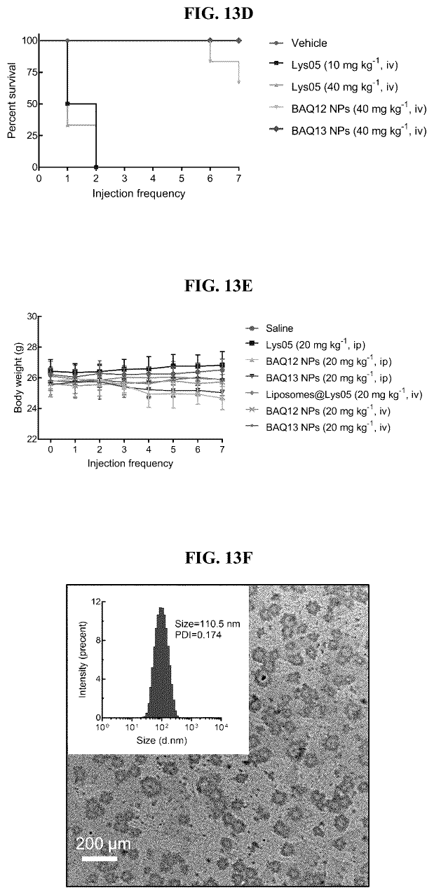

Pharmacokinetics, biodistribution, and toxicity. The pharmacokinetics of BAQ ONNs were studied in Sprague-Dawley rats upon intravenous (iv) injection. As shown in FIG. 5A and Table 5, the serum concentrations of BAQ ONNs were higher than those of free DiD at the same time points up to 48 h, indicating that the plasma clearance of BAQ ONNs was slower than that of DiD because of the nanoscale characteristics of BAQ ONNs. DiD-labelled BAQ13 NPs were also used to investigate the biodistribution of the NPs in nude mice bearing HT29 tumours. As expected, both in vivo and ex vivo imaging showed that the fluorescence signals of BAQ13 NPs were clearly distinguishable in tumour areas rather than in surrounding normal tissues at 12 h and 24 h post-injection, indicating the tumour-targeting biodistribution of BAQ ONNs (FIGS. 5B-5C and FIGS. 13A-13B). This targeting ability may be due to the relatively high permeabilization of tumour blood vessels, which enables passive accumulation of nanotherapeutics. The free DiD (control) group showed high signals in the lungs, rather than in the tumour sites (FIGS. 13A-13B). In particular, the tumour-to-lung ratio of fluorescence in the BAQ13 NP group was ˜4-fold higher than that in the free DiD group. These results demonstrated that BAQ ONNs had a tumour-targeting biodistribution.

TABLE 5
The calculated pharmacokinetic parameters using Kinetica 5.0.

| Nanoparticles | Cmax (ng mL⁻¹) | AUC₀₋₄₈ | T_{1/2} (h) |
|---|---|---|---|
| Free DiD | 8.6 | 34.9 | 0.8 |
| BAQ12 | 10.6 | 124.7 | 12.4 |
| NPs@DiD | | | |
| BAQ13 | 11.0 | 182.0 | 15.9 |
| NPs@DiD | | | |

Next a haemolysis assay was carried out to evaluate the safety of BAQ ONNs. Under physiological conditions, red blood cells were treated with Lys05, BAQ12 NPs or BAQ13 NPs at concentrations of 0.25-1 mg mL⁻¹, close to the working concentrations used for the animal treatment study (FIG. 13C). Treatment with the control drug Lys05 resulted in a significantly higher haemolytic rate than treatment with BAQ12 NPs or BAQ13 NPs at concentrations above 0.5 mg mL⁻¹, indicating the greater safety of BAQ ONNs than Lys05.

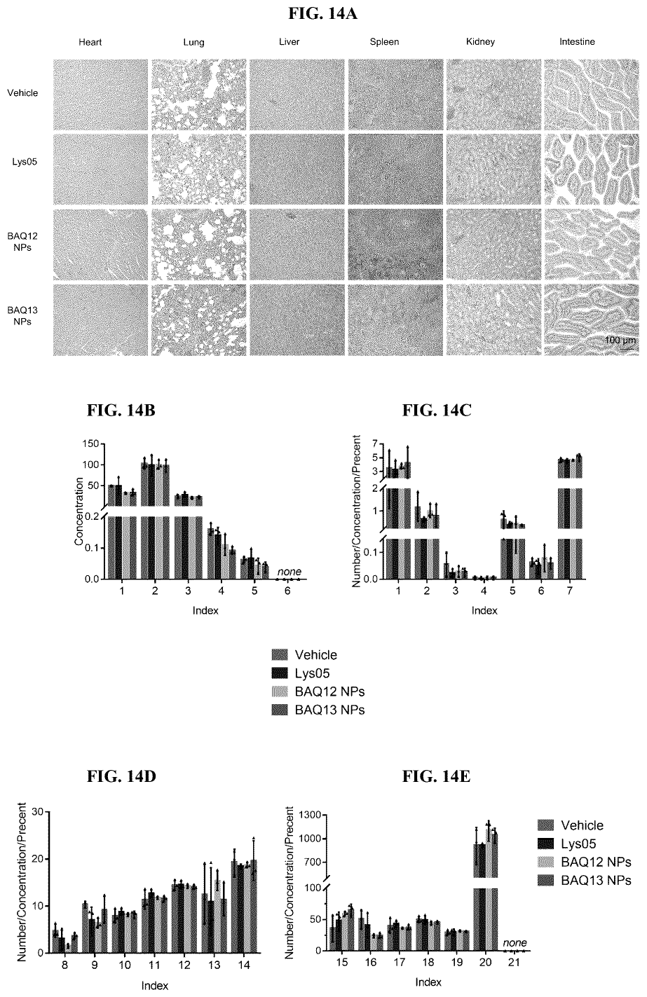

In the following animal toxicity studies on FVB/N mice, Lys05 treatment was found to cause acute death of mice after iv administration even at a low concentration of 10 mg kg⁻¹; in contrast, BAQ ONN treatment resulted in low mortality and no body weight loss, revealing that BAQ ONNs are safe when administered via iv injection (FIGS. 13D-13E). Since they did not lead to any death at 40 mg kg⁻¹, BAQ13 NPs were better tolerated by the mice than BAQ12 NPs. This result is likely attributable to the high stability and low CAC of BAQ13 NPs. Liposomes were used to encapsulate Lys05 (liposomes@Lys05) and found that this formulation is safe for iv injection (FIGS. 13E-13F). Because autophagy plays an important role in intestinal homeostasis, intraperitoneal (ip) administration of BAQ12 NPs or BAQ13 NPs, which results in an increased autophagy-inhibiting effect, may cause intestinal disorders and loss of body weight in mice. H&E staining of tissue sections and haematologic indexes did not show obvious abnormal alterations in mice treated with 20 mg kg⁻¹ BAQ NPs by tail vein, which further suggested that iv administration of BAQ ONNs is well tolerated (FIGS. 14A-14E). Therefore, BAQ ONNs should be administered by iv injection rather than by ip injection for investigation of their advantages in vivo.

Antitumour effect as single agents in mice. After proving the safety of BAQ ONNs, the NPs for anticancer efficacy was evaluated in a pancreatic xenograft model of MIA PaCa-2 cells. NRG mice with MIA PaCa-2 tumours (˜100 mm³) were randomly assigned into five groups (n=6): the saline (iv) group, the Lys05 (ip) group, the liposomes@Lys05 (iv) group, the BAQ12 NPs (iv) group and the BAQ13 NPs (iv) group. The mice were then treated every three days at a dose of 20 mg kg⁻¹. The results in FIGS. 5D-5F show that the treatment with BAQ12 NPs or BAQ13 NPs significantly decelerated tumour growth without interfering with body weight. The control drug Lys05 did not display a therapeutic effect under this condition, but its nanoformulation, liposomes@Lys05, elicited increased tumour inhibition, which highlights the advantage of nanomedicines in drug delivery. It should also be emphasized that the one-component formulations of self-assembling BAQ12 NPs or BAQ13 NPs were significantly more efficacious than either free Lys05 or nanoformulated Lys05. These findings clearly illustrate the potential advantages of BAQ ONNs with regard to both drug discovery and drug delivery.

To further understand the in vivo effects of BAQ ONNs, tumour tissues were harvested for histological assessment. Dramatic cellular destruction, increased cleaved caspase-3 levels and decreased Ki67 expression were observed in both BAQ ONN groups, suggesting that the tumours treated with BAQ ONNs were inclined to die or to become apoptotic or quiescent (FIGS. 5G-5H). LC3 expression was increased in both BAQ ONN groups (FIG. 5H); this finding is an essential clue explaining the in vivo autophagy-inhibiting effects of both BAQ ONNs. The subsequent immunoblot analysis further demonstrated that autophagy in tumours was blocked by the BAQ ONNs (FIG. 5I). Additionally, the tissue ultrastructure was observed by TEM and found that BAQ ONN-treated tumours contained more numerous large AVs than the groups of tumours (FIG. 5J). In the assays above, the Lys05 nanoformulation, liposomes@Lys05, also exhibited some effects not observed with the vehicle or free Lys05. However, the effects of liposomes@Lys05 were much weaker than those of either BAQ12 NPs or BAQ13 NPs. These tissue-level results revealed the excellent autophagy-inhibiting effects of BAQ ONNs in vivo.

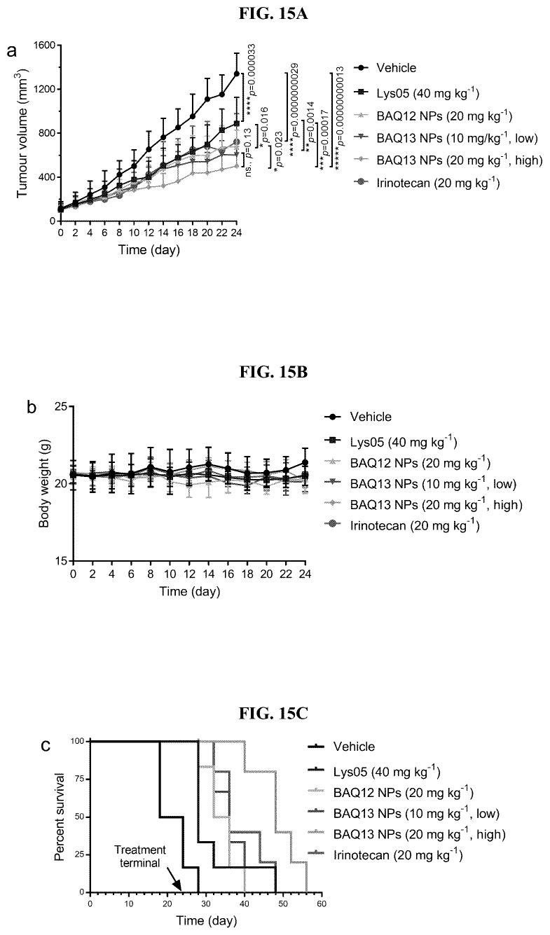

The therapeutic effects of BAQ ONNs in vivo were further demonstrated in another animal model consisting of mice bearing colon HT29 tumours. Compared with vehicle or Lys05 administration, BAQ ONN administration significantly inhibited tumour growth (FIGS. 15A-15B). And BAQ13 NPs displayed better efficacy than BAQ12 NPs. Interestingly, this result contradicted the results obtained in the in vitro proliferation assay, which demonstrated BAQ12 NPs as being more effective than BAQ13 NPs. This discrepancy could be explained by the differences in self-assembly behaviours and pharmacokinetic profiles between the BAQ ONNs (FIG. 2G, FIG. 5A). Moreover, BAQ13 NPs were also more efficacious than FDA-approved irinotecan at its reported therapeutic dose, while BAQ12 NPs showed effects similar to those of irinotecan. Survival analysis revealed that BAQ13 NP treatment resulted in a significantly longer survival time (median survival of 48 days) than vehicle and irinotecan groups (median survival of 21 or 36 days, respectively) (FIG. 15C and Table 6). Given their integration of multiple advantages regarding both pharmacodynamic effects and pharmacokinetic profiles, the hybrid BAQ ONNs exhibit enormous potential for cancer treatment in vivo as single agents.

TABLE 6
Median survival of mice bearing HT29
tumours. n = 6 mice per group.

| Groups | Median Survival (day) |
|---|---|
| Vehicle | 21 |
| Lys05 | 28 |
| BAQ12 NPs | 34 |
| BAQ13 NPs (low) | 36 |
| BAQ13 NPs (high) | 48 |
| Irinotecan | 36 |

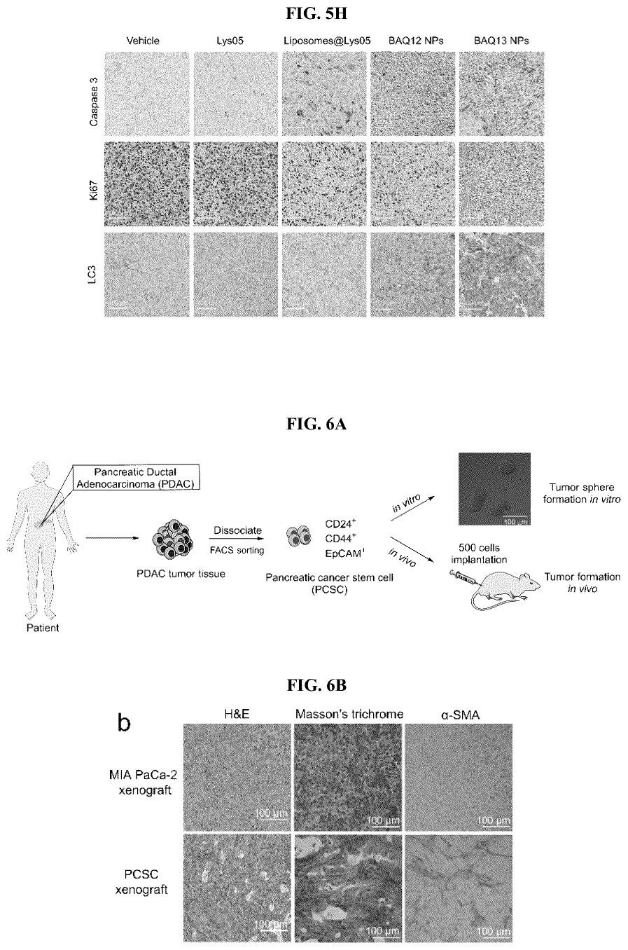

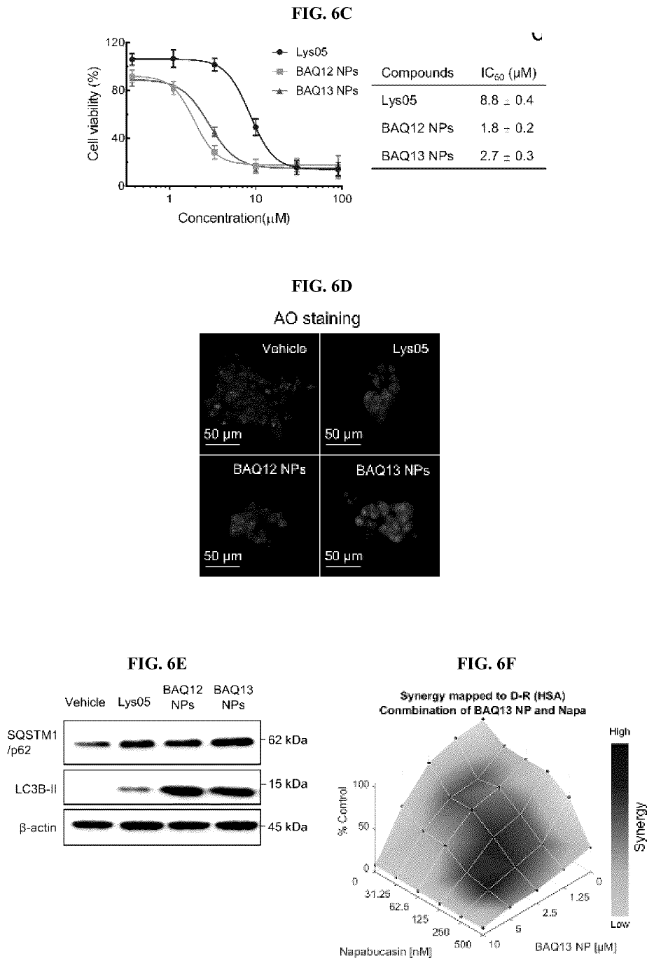

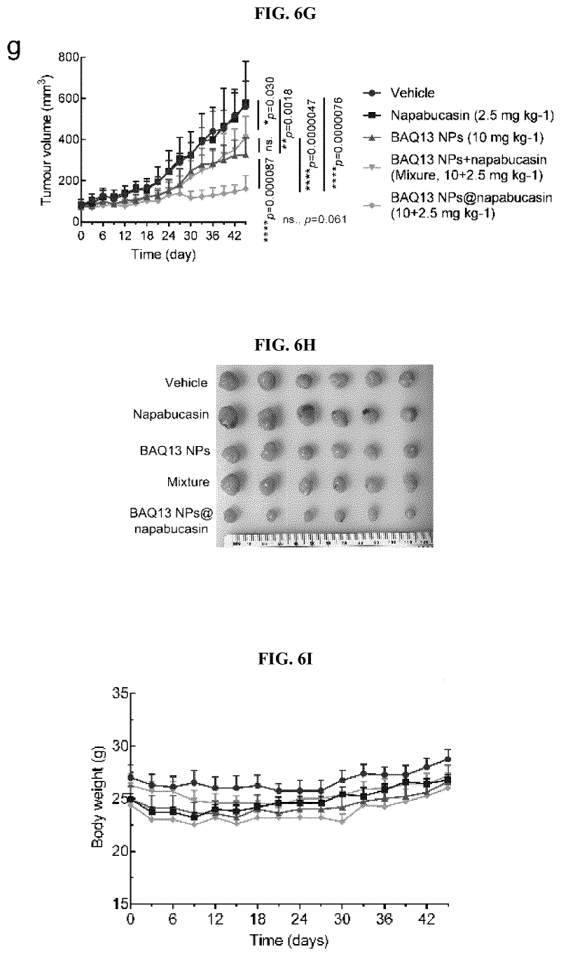

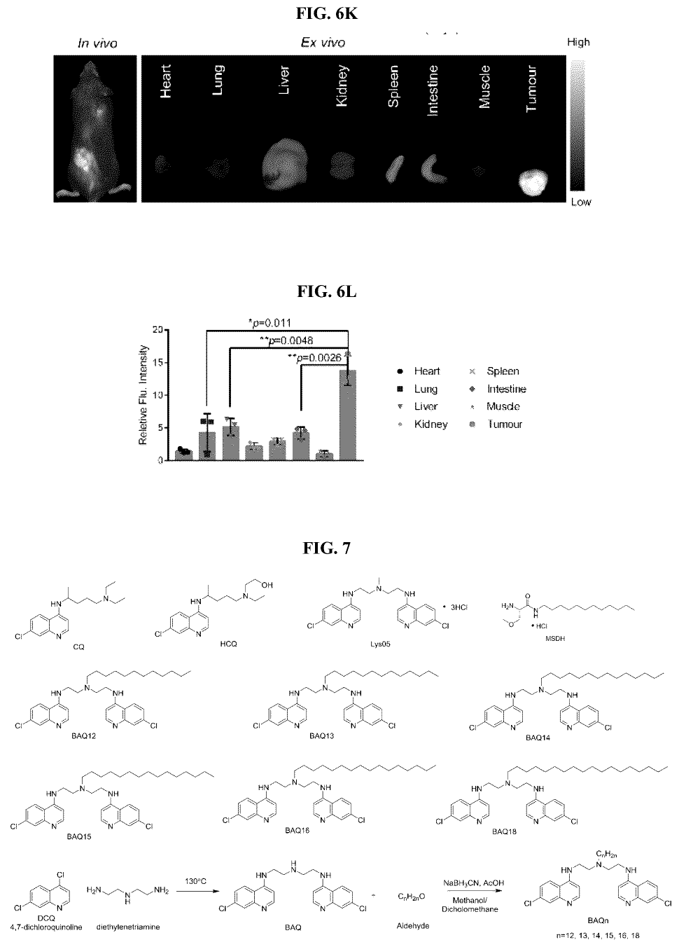

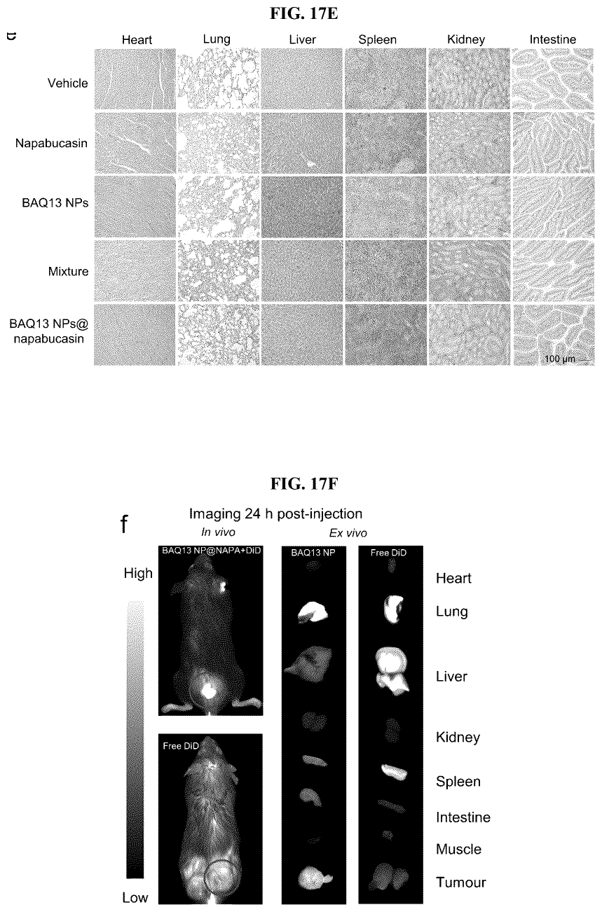

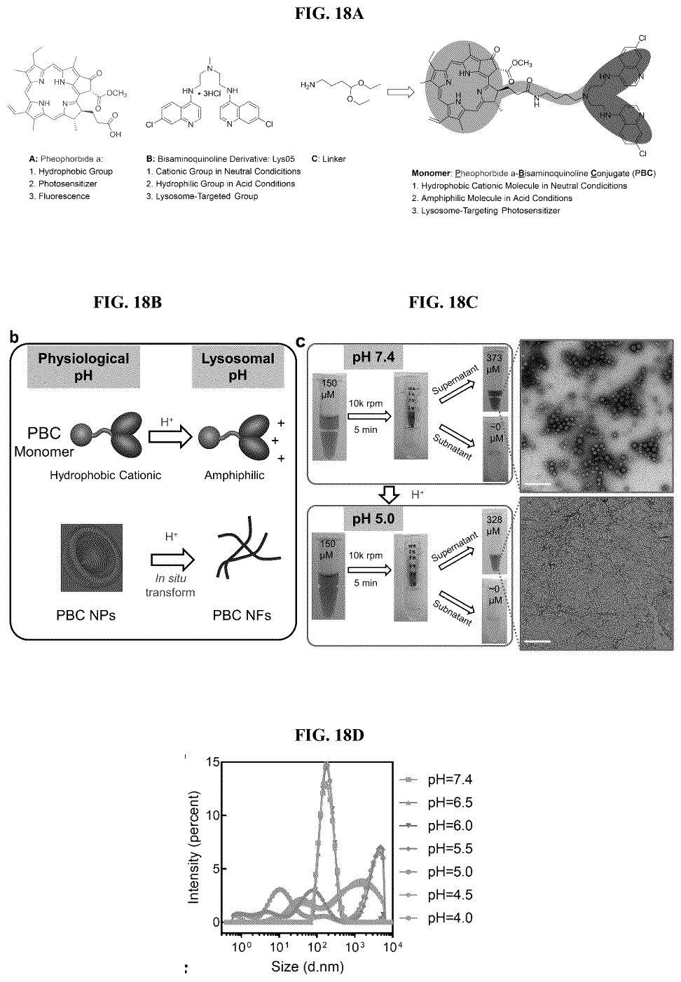

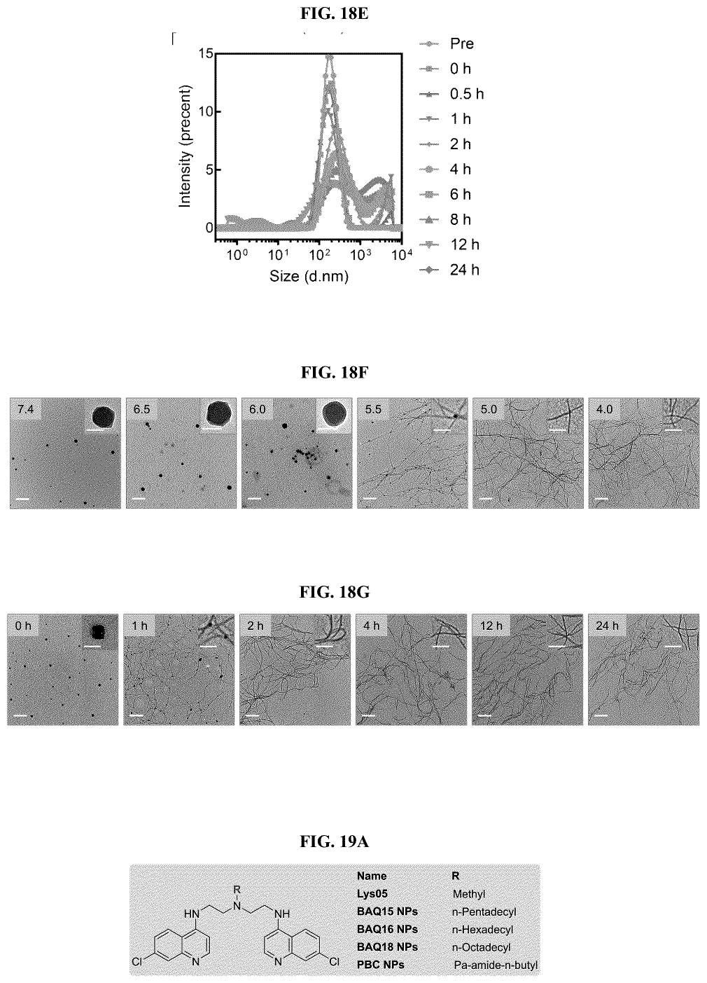

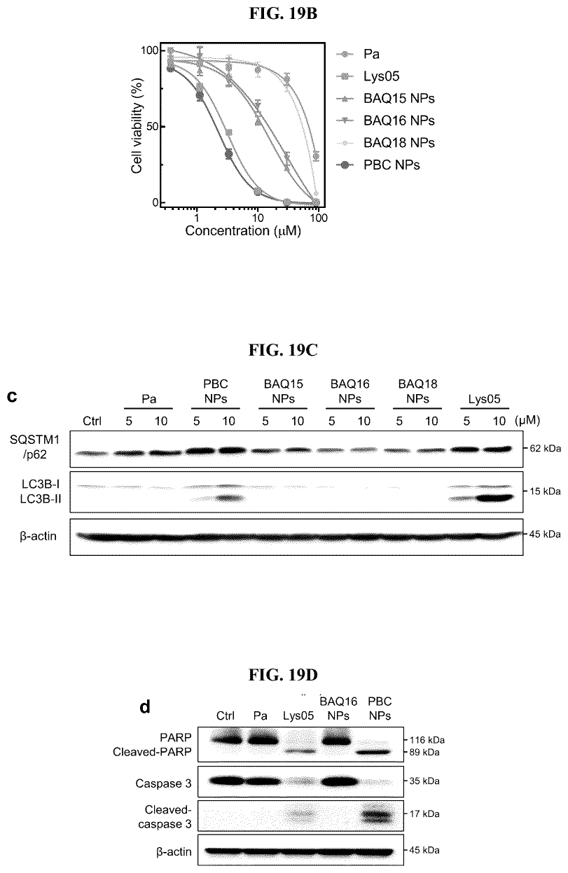

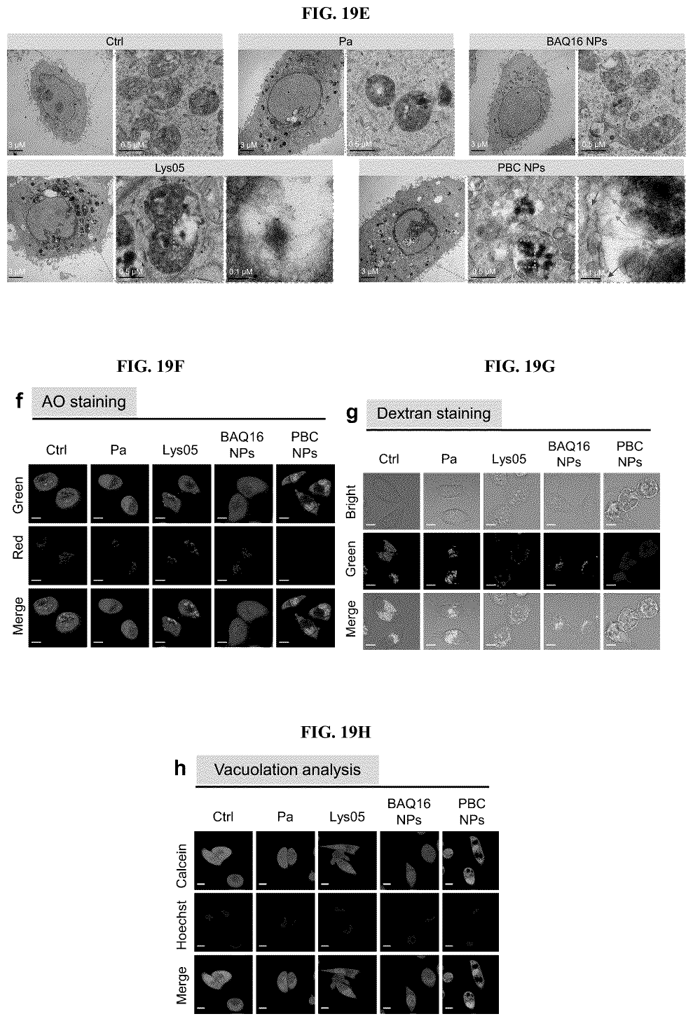

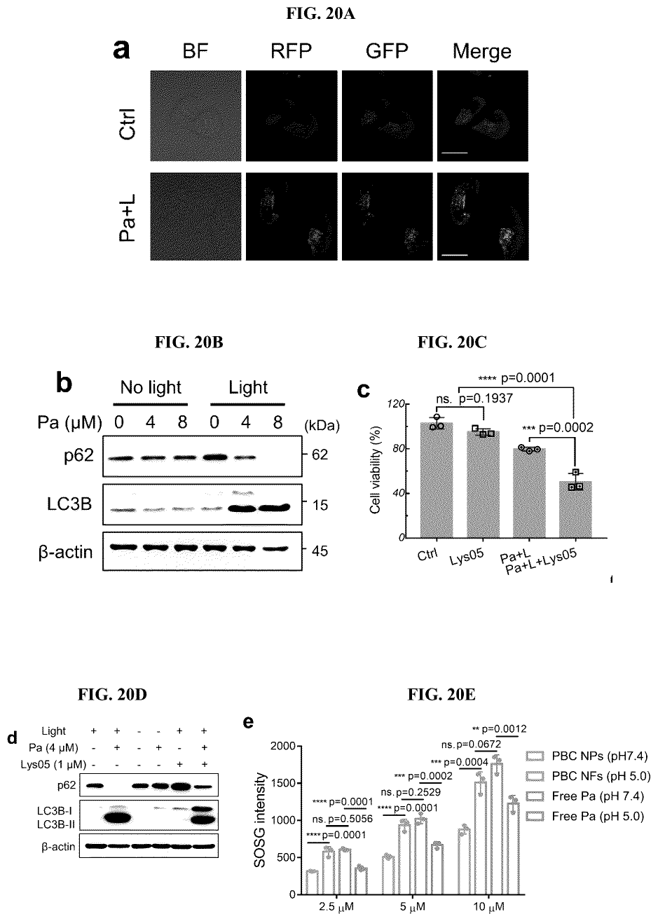

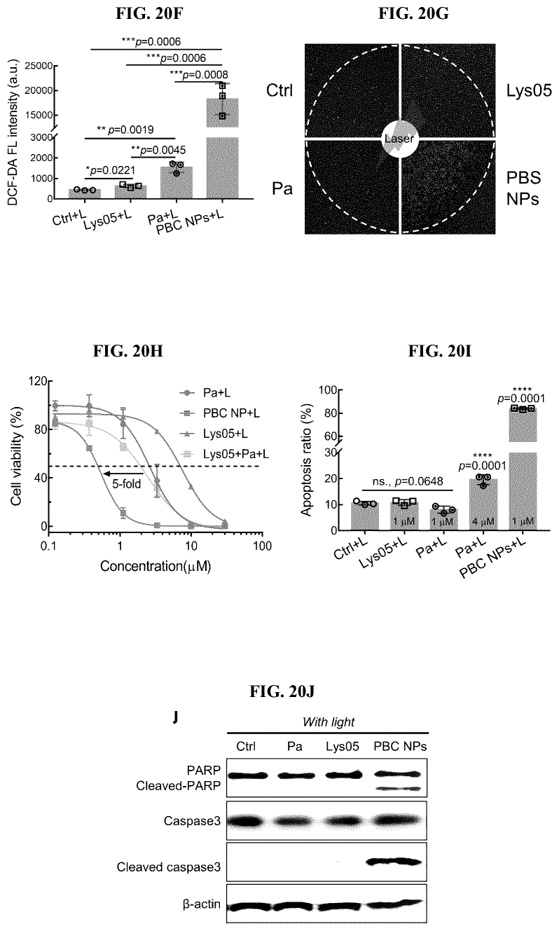

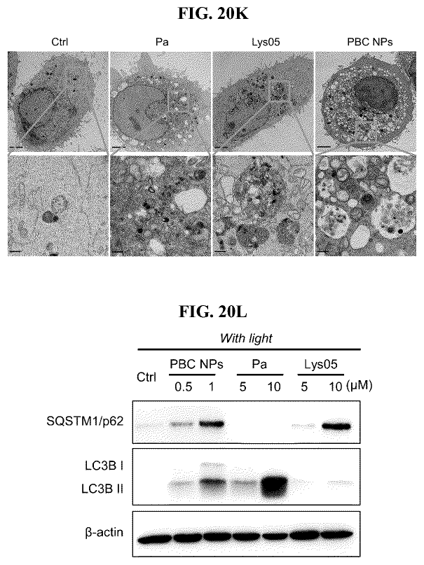

Dual roles of BAQ ONNs in combination therapy. Autophagy inhibition-based combination therapy could sensitize tumours to conventional therapeutics, but the current limitation is the insufficient efficacy of autophagy inhibitors. Moreover, the disparate pharmacokinetics and different dosing schedules of drugs used in combination therapy are inconvenient. Given the 30-fold higher anticancer potency of BAQ ONNs than HCQ and their considerable potential to encapsulate additional drugs, BAQ ONNs may be able to address these two pharmacodynamic and pharmacokinetic issues simultaneously. To test this hypothesis, a xenograft model with high heterogeneity and a high tumour stroma proportion by using a pancreatic cancer stem cell (PCSC) line from patient-derived pancreatic adenocarcinoma tissue was established (FIGS. 6A-6B and FIG. 16). The in vitro results proved that BAQ ONNs had similar functions in inhibiting lysosomes and autophagy in PCSCs and therefore exhibited potent proapoptotic and antiproliferative activities (FIGS. 6C-6E and FIGS. 17A-17C). The STAT3 inhibitor napabucasin, which can be encapsulated in BAQ13 NPs, was chosen for the combination therapy because it can induce autophagy and synergize with BAQ13 NPs (FIG. 6F and FIG. 17D). Mice were randomly divided into 5 groups, including the vehicle (saline) group, the napabucasin group, the BAQ13 NPs group, the mixture (BAQ13 NPs+napabucasin) group and the BAQ13 NPs@napabucasin group (FIGS. 6G-6I). BAQ13 NPs moderately inhibited tumour growth, while napabucasin itself exhibited no antitumour effect under these conditions. The mixture group did not exhibit an enhanced effect in vivo, although in vitro synergy of napabucasin and BAQ13 NPs was observed. This lack of in vivo effect was probably due to the poor solubility and inefficient delivery of napabucasin. When loaded in BAQ13 NPs (BAQ13 NPs@Napabucasin), the nanoformulated napabucasin achieved a satisfactory antitumour effect by synergizing with BAQ13 NPs. Remarkable changes in tumour histology were also observed in the BAQ13 NPs@napabucasin group, in which the cells showed low proliferation activity (FIG. 6J). In addition, none of treatment groups of mice exhibited obvious systemic toxicity (FIG. 61 and FIG. 17E). To further verify the ability of BAQ13 NPs to deliver napabucasin, another imaging study was performed on the PCSC model by using DiD-labelled BAQ13 NPs@napabucasin. The results showed obvious accumulation of NPs in tumour sites rather than normal organs (FIGS. 6K-6L and FIG. 17F). These interesting results demonstrate that BAQ13 NPs can function not only as therapeutic agents but also as delivery carriers in combination therapy; therefore, they show promise for improving cancer treatment.

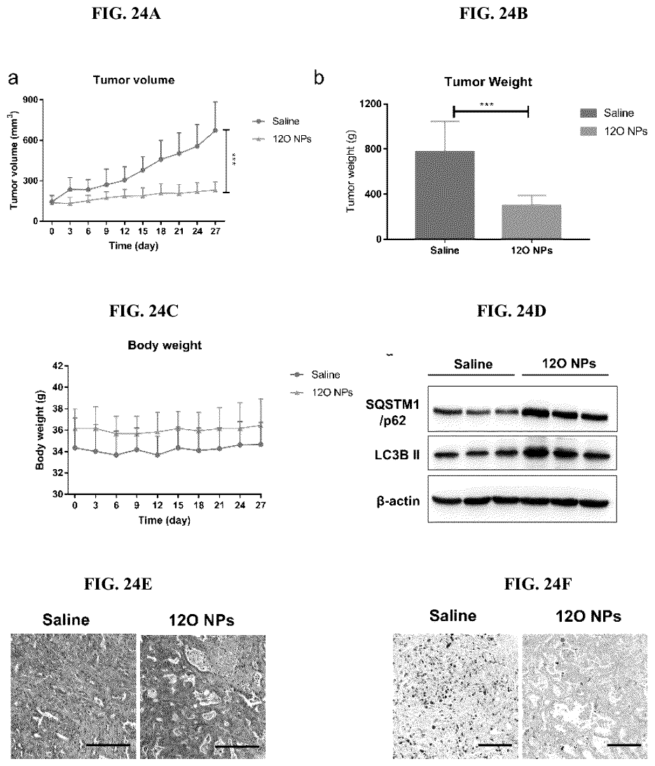

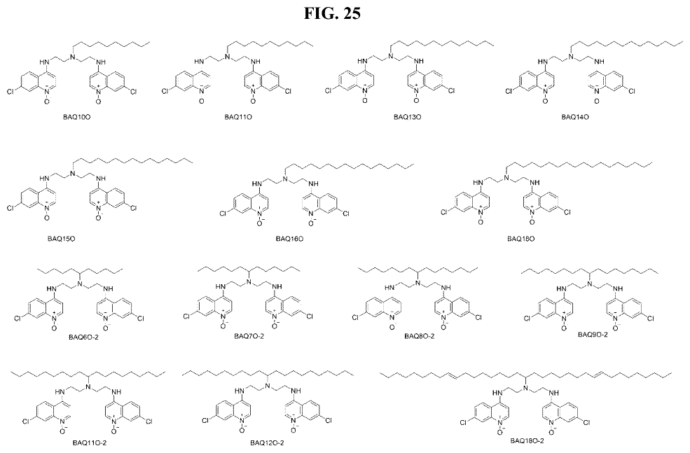

Antitumour effect of BAQO derivatives in mice. BAQO derivatives can form nanoparticles and be used for in vivo mice studies. For example, BAQ120 NPs were used to treat mice bearing PCSC tumors. As shown in FIG. 24A, mice treated with BAQ100 NPs has lower tumor volume, with a significant difference in tumor volume by day 27. FIG. 24B shows that the tumor weight at the end of treatment with BAQ120 NPs was 50% less than the tumor weight of the control. These interesting results demonstrate that BAQO derivatives can function as promising agents for drug discovery and drug delivery.

Based on an ONN strategy and the principles of pharmacophore hybridization and molecular self-assembly, the self-delivering new chemical entities, BAQ ONNs, was developed. These entities were equipped with enhanced abilities to induce lysosomal disruption, lysosomal dysfunction and autophagy blockade in addition to improved properties for drug delivery and tumour-targeted biodistribution; thus, they exhibited significant anticancer efficacy both in vitro and in vivo. Strikingly, it was found that the simple BAQ13 NPs showed high drug-loading efficiency and could potently synergize with and deliver an additional drug, thus showing promise for application in combination therapy.

In contrast to conventional NPs, which typically have an active pharmaceutical ingredient (API) content of less than 20% and are complicated to synthesize, BAQ ONNs have a 100% API content and are easy to synthesize and scale up. Since they are non-prodrug chemical entities, they are also superior to emerging one-component prodrug NPs. All these advantages will greatly facilitate their translation into clinical trials. This is an important attempt to extend nanotechnology into the design of new chemical entities. A seamless connection between drug discovery and nanotechnology-assisted drug delivery will enable researchers to develop increasingly advanced nanomedicines with a wide range of therapeutic and commercial benefits for cancer targeting.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound of formula (I): 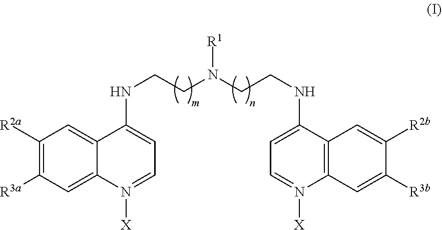
or a pharmaceutically acceptable salt thereof, wherein:
R¹ is hydrogens, C₁₋₄₀ alkyl, C₂₋₄₀ alkenyl, C₂₋₄₀ alkynyl, —W, -(L-Y)ₚ—Z, or —C(O)R^{1a}, wherein each alkyl, alkenyl and alkynyl are optionally substituted with C₁₋₂₀ alkoxy, hydroxyl, or —NR^{1b}R^{1c};
W is C₃₋₁₂ cycloalkyl, C₆₋₁₂ aryl, or a 5 to 12 membered heteroaryl having 1 to 4 heteroatoms each independently N, O, or S, and wherein each cycloalkyl, aryl, and heteroaryl are optionally substituted with C₁₋₄₀ alkyl, C₂₋₄₀ alkenyl, or C₂₋₄₀ alkynyl;
each L is independently absent, C₁₋₂₀ alkylene, C₂₋₂₀ alkenylene, or C₂₋₂₀ alkynylene;
each Y is independently absent, —O—, —NH—, —NHC(O)—, —NHC(O)NH—, —NHSO₂—, —OC(O)—, —OC(O)NH—, —C(O)—, or —SO₂—;
Z is a fluorophore, a photosensitizer, a porphyrin, a chemotherapeutic drug, a sterol, C₃₋₁₂ cycloalkyl, 3 to 12 membered heterocycloalkyl having 1 to 4 heteroatoms each independently N, O or S, C₆₋₁₂ aryl, 5 to 12 membered heteroaryl having 1 to 4 heteroatoms each independently N, O or S, —OH, or —NH₂;
R^{1a} is C₁₋₄₀ alkyl, C₂₋₄₀ alkenyl, or C₂₋₄₀ alkynyl, wherein each alkyl, alkenyl and alkynyl are optionally substituted with C₁₋₄₀ alkoxy, hydroxyl, or —NR^{1b}R^{1c};
R^{1b} is hydrogen, C₁₋₄₀ alkyl, C₂₋₄₀ alkenyl, or C₂₋₄₀ alkynyl;
R^{1c} is hydrogen, C₁₋₄₀ alkyl, C₂₋₄₀ alkenyl, C₂₋₄₀ alkynyl, or -L-W;
R^{2a}, R^{2b}, R^{3a}, and R^{3b} are each independently hydrogen, C₁₋₄₀ alkyl, C₂₋₄₀ alkenyl, C₂₋₄₀ alkynyl, C₁₋₄₀ alkoxy, halogen, —CN, or —NO₂;
m and n are independently an integer from 1 to 10;
p is independently an integer from 1 to 20; and
each X is independently absent or —O—,
wherein when X is absent, R^{2a} and R^{2b} are hydrogen, and R^{3a} and R^{3b} are each independently hydrogen, -OMe, fluorine, chlorine, bromine, or —NO₂, then R¹ is C₂₋₄₀ alkyl, C₂₋₄₀ alkenyl, C₄₋₄₀ alkynyl, —W, -(L-Y)ₚ—Z, or —C(O)R^{1a}, and
wherein when X is absent, R¹ is —CH₂CH₂NH(7-chloro-4-quinolinyl), and R^{2a} and R^{2b} are hydrogen, then R^{3a} and R^{3b} are independently selected from hydrogen, C₁₋₂₀ alkyl, C₂₋₄₀ alkenyl, C₂₋₄₀ alkynyl, C₁₋₄₀ alkoxy, fluorine, bromine, iodine, —CN, or —NO₂.

2. The compound of claim 1, or a pharmaceutically acceptable salt thereof, wherein
R¹ is hydrogen, C₁₋₂₀ alkyl, C₂₋₂₀ alkenyl, C₂₋₂₀ alkynyl, —W, -(L-Y)ₚ—Z, or —C(O)R^{1a}, wherein each alkyl, alkenyl and alkynyl are optionally substituted with C₁₋₂₀ alkoxy, hydroxyl, or —NR^{1b}R^{1c};
W is C₃₋₁₂ cycloalkyl, C₆₋₁₂ aryl, or 5 to 12 membered heteroaryl, wherein the 5 to 12 membered heteroaryl have 1 to 4 heteroatoms of N, O, and S, and wherein each cycloalkyl, aryl, and heteroaryl are optionally substituted with C₁₋₂₀ alkyl, C₂₋₂₀ alkenyl, or C₂₋₂₀ alkynyl;
each L is independently absent, C₁₋₁₀ alkylene, C₂₋₁₀ alkenylene, or C₂₋₁₀ alkynylene;
each Y is independently absent, —O—, —NH—, —NHC(O)—, —NHC(O)NH—, —NHSO₂—, —OC(O)—, —OC(O)NH—, —C(O)—, or —SO₂—;
Z is a fluorophore, a photosensitizer, a porphyrin, a chemotherapeutic drug, or a sterol;
R^{1a} is C₁₋₂₀ alkyl, C₂₋₂₀ alkenyl, or C₂₋₂₀ alkynyl, wherein each alkyl, alkenyl and alkynyl are optionally substituted with C₁₋₂₀ alkoxy, hydroxyl, or —NR^{1b}R^{1c};
R^{1b} is hydrogen, C₁₋₂₀ alkyl, C₂₋₂₀ alkenyl, or C₂₋₂₀ alkynyl;
R^{1c} is hydrogen, C₁₋₂₀ alkyl, C₂₋₂₀ alkenyl, C₂₋₂₀ alkynyl, or -L-W;
R^{2a}, R^{2b}, R^{3a}, and R^{3b} are each independently hydrogen, C₁₋₂₀ alkyl, C₂₋₂₀ alkenyl, C₂₋₂₀ alkynyl, C₁₋₂₀ alkoxy, halogen, —CN, or —NO₂;
m and n are independently an integer from 1 to 10;
p is independently an integer from 1 to 20; and
each X is independently absent or —O—,
wherein when X is absent, R^{2a} and R^{2b} are hydrogen, and R^{3a} and R^{3b} are each independently hydrogen, -OMe, fluorine, chlorine, bromine, or —NO₂, then R¹ is C₂₋₂₀ alkyl, C₂₋₂₀ alkenyl, C₄₋₂₀ alkynyl, —W, -(L-Y)ₚ—Z, or —C(O)R^{1a}, and
wherein when X is absent, R¹ is —CH₂CH₂NH(7-chloro-4-quinolinyl), and R^{2a} and R^{2b} are hydrogen, then R^{3a} and R^{3b} are independently selected from hydrogen, C₁₋₂₀ alkyl, C₂₋₂₀ alkenyl, C₂₋₂₀ alkynyl, C₁₋₂₀ alkoxy, fluorine, bromine, iodine, —CN, or —NO₂.

3. The compound of claim 1, or a pharmaceutically acceptable salt thereof, wherein R¹ is C₁₋₄₀ alkyl, C₂₋₄₀ alkenyl, -(L-Y)ₚ—Z, or —C(O)R^{1a}.

4. The compound of any one of claims 1 to 3, or a pharmaceutically acceptable salt thereof, wherein R¹ is C₁₋₂₅ alkyl.

5. The compound of any one of claim 1 to 4, or a pharmaceutically acceptable salt thereof, wherein R¹ is C₁₀₋₂₅ alkyl.

6. The compound of any one of claims 1 to 5, or a pharmaceutically acceptable salt thereof, wherein R¹ is C₁₂₋₁₈ alkyl.

7. The compound of any one of claims 1 to 3, or a pharmaceutically acceptable salt thereof, wherein R¹ is C₂₀₋₄₀ alkenyl.

8. The compound of claim 7, or a pharmaceutically acceptable salt thereof, wherein R¹ is C₃₋₄₀ alkenyl.

9. The compound of any one of claims 1 to 3, or a pharmaceutically acceptable salt thereof, wherein R¹ is -(L-Y)ₚ—Z.

10. The compound of claim 9, or a pharmaceutically acceptable salt thereof, wherein p is 1.

11. The compound of claim 9 or 10, or a pharmaceutically acceptable salt thereof, wherein L is C₁₋₁₀ alkylene.

12. The compound of any one of claims 9 to 11, or a pharmaceutically acceptable salt thereof, wherein Y is absent, —NH—, —NHC(O)— or —NHC(O)NH—

13. The compound of any one of claims 9 to 12, or a pharmaceutically acceptable salt thereof, wherein Z is a porphyrin, a sterol, 6 to 12 membered heterocycloalkyl, 8 to 12 membered heteroaryl, —OH, or —NH₂, wherein the 6 to 12 membered heterocycloalkyl and the 8 to 12 membered heteroaryl have 1 to 4 heteroatoms of N, O, and S.

14. The compound of any one of claims 9 to 13, or a pharmaceutically acceptable salt thereof, wherein:
p is 1;
L is C₄₋₅ alkylene;
Y is absent, —NH—, or —NHC(O)—; and
Z is porphyrin, cholic acid, isoindoline, phthalimide, —OH, or —NH₂.

15. The compound of any one of claims 1 to 3, or a pharmaceutically acceptable salt thereof, wherein R¹ is —C(O)R^{1a}.

16. The compound of claim 15, or a pharmaceutically acceptable salt thereof, wherein R^{1a} is C₁₋₁₀ alkyl.

17. The compound of any one of claims 1 to 16, or a pharmaceutically acceptable salt thereof, wherein R^{2a} and R^{2b} are each independently hydrogen, fluorine, chlorine, bromine, or iodine.

18. The compound of any one of claims 1 to 17, or a pharmaceutically acceptable salt thereof, wherein R^{2a} and R^{2b} are each independently hydrogen.

19. The compound of any one of claims 1 to 18, or a pharmaceutically acceptable salt thereof, wherein R^{3a} and R^{3b} are each independently hydrogen, fluorine, chlorine, bromine, or iodine.

20. The compound of any one of claims 1 to 19, or a pharmaceutically acceptable salt thereof, wherein R^{3a} and R^{3b} are each independently chlorine.

21. The compound of any one of claims 1 to 20, or a pharmaceutically acceptable salt thereof, wherein m and n are each independently an integer 1 to 5.

22. The compound of any one of claims 1 to 21, or a pharmaceutically acceptable salt thereof, wherein m and n are each independently 1.

23. The compound of any one of claims 1 to 22, or a pharmaceutically acceptable salt thereof, wherein the compound is the compound of Formula (Ia):

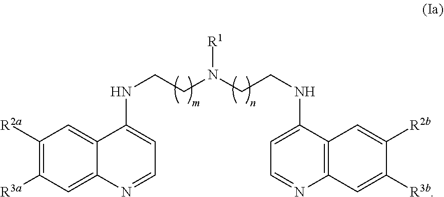

24. The compound of any one of claims 1 to 6 or 17 to 23, or a pharmaceutically acceptable salt thereof, wherein the compound has the structure: 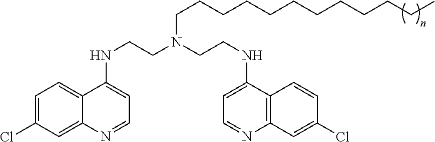
wherein n is an integer from 1 to 7.

25. The compound of any one of claims 1 to 6 or 17 to 24, or a pharmaceutically acceptable salt thereof, wherein the compound is selected from the group consisting of:

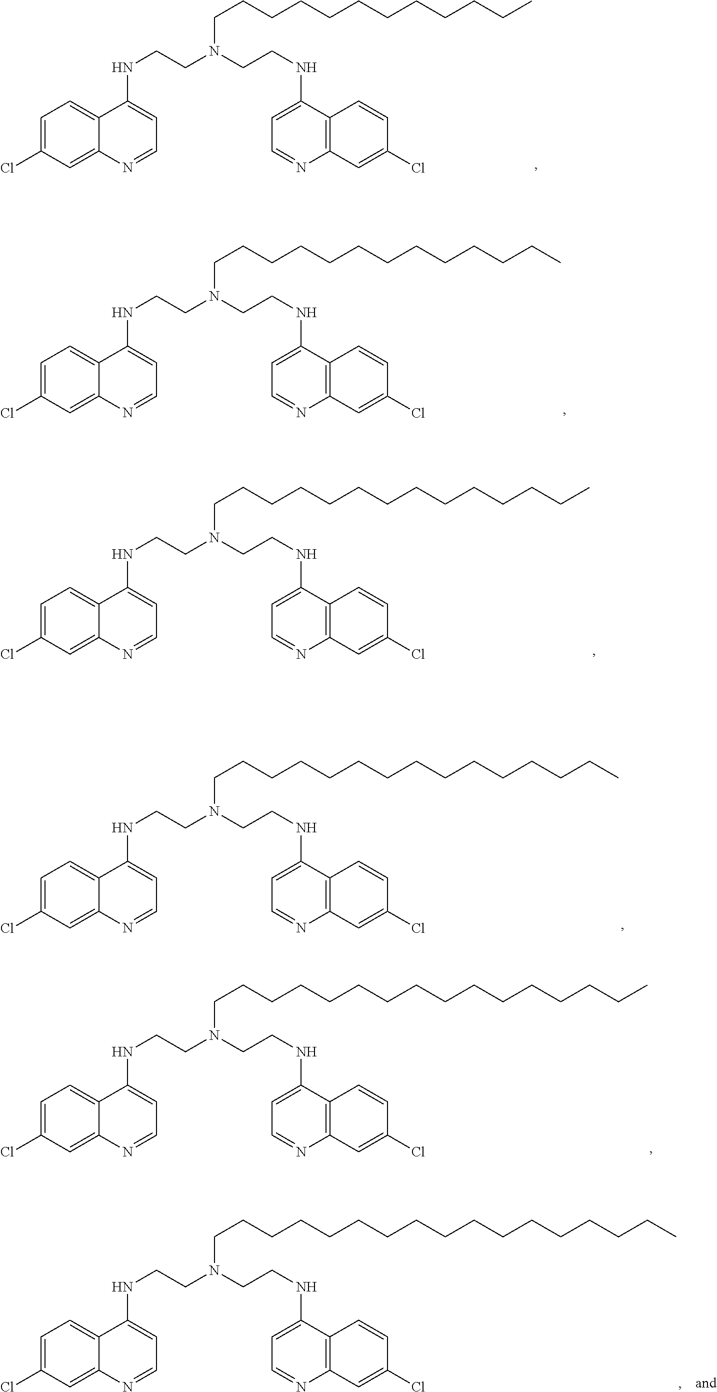 [SMILES: CCCCCCCCCCCCN(CCNc1ccnc2cc(Cl)ccc12)CCNc1ccnc2cc(Cl)ccc12 ; CCCCCCCCCCCCCN(CCNc1ccnc2cc(Cl)ccc12)CCNc1ccnc2cc(Cl)ccc12 ; CCCCCCCCCCCCCCN(CCNc1ccnc2cc(Cl)ccc12)CCNc1ccnc2cc(Cl)ccc12 ; CCCCCCCCCCCCCCCN(CCNc1ccnc2cc(Cl)ccc12)CCNc1ccnc2cc(Cl)ccc12 ; CCCCCCCCCCCCCCCCN(CCNc1ccnc2cc(Cl)ccc12)CCNc1ccnc2cc(Cl)ccc12 ; CCCCCCCCCCCCCCCCCN(CCNc1ccnc2cc(Cl)ccc12)CCNc1ccnc2cc(Cl)ccc12]

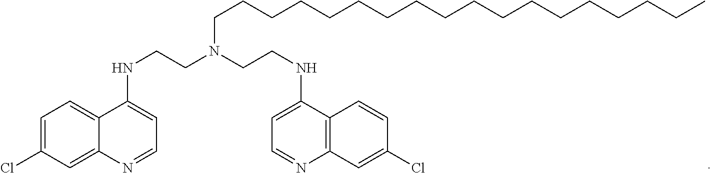 [SMILES: CCCCCCCCCCCCCCCCCCN(CCNc1ccnc2cc(Cl)ccc12)CCNc1ccnc2cc(Cl)ccc12]

26. The compound of any one of claims 1 to 3, 9 to 14, or 17 to 23, or a pharmaceutically acceptable salt thereof, wherein the compound is

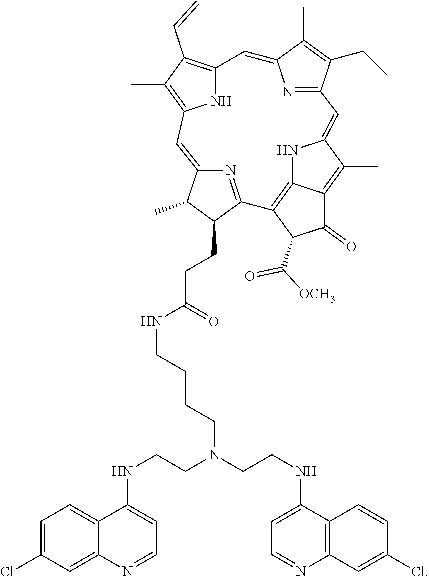 [SMILES: C=Cc1c(C)c2cc3nc(c4c5[nH]c(cc6nc(cc1[nH]2)C(C)=C6CC)c(C)c5C(=O)[C@@H]4C(=O)OC)[C@@H](CCC(=O)NCCCCN(CCNc1ccnc2cc(Cl)ccc12)CCNc1ccnc2cc(Cl)ccc12)[C@@H]3C]

27. The compound of any of claims 1 to 3, 9 to 14, or 17 to 23, or a pharmaceutically acceptable salt thereof, wherein the compound is selected from the group consisting of:

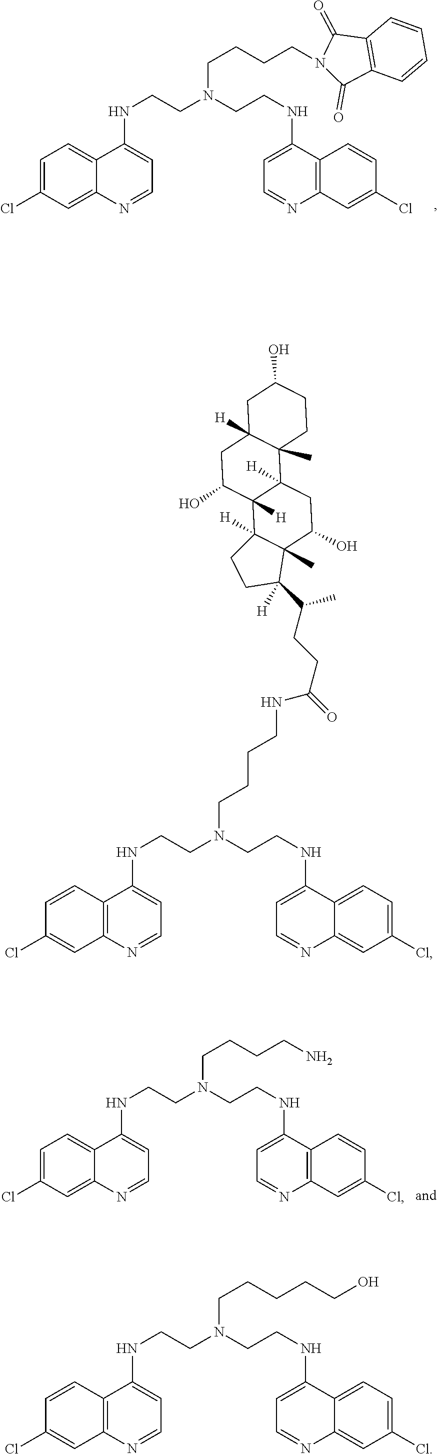

28. The compound of any one of claims 1 to 3 or 4 to 22, or a pharmaceutically acceptable salt thereof, wherein the compound is the compound of Formula (Ib):

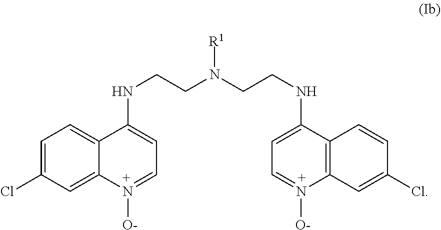

29. The compound of claim 28, or a pharmaceutically acceptable salt thereof, wherein the compound is selected from the group consisting of:

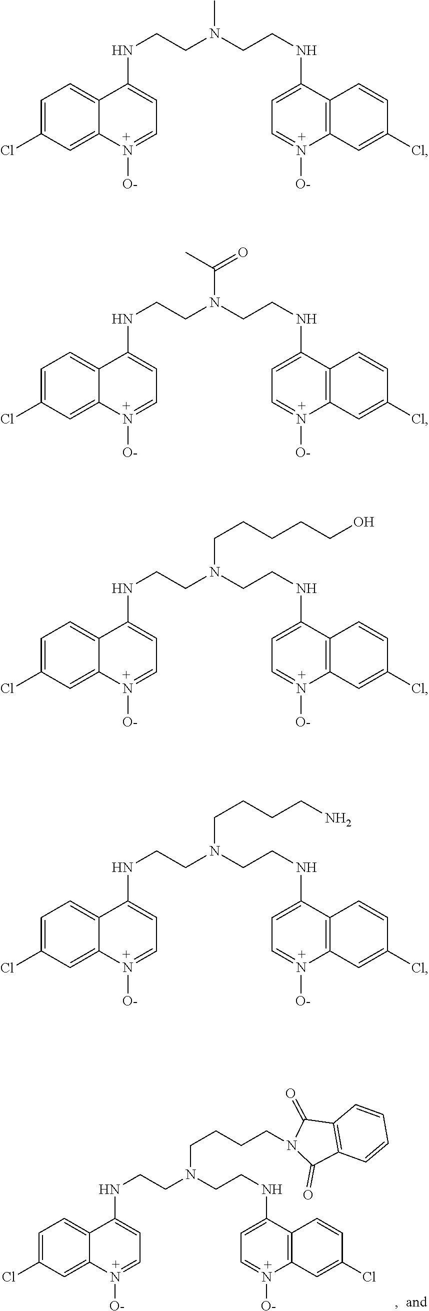

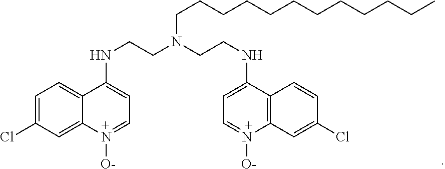

30. The compound of claim 28, or a pharmaceutically acceptable salt thereof, wherein the compound is selected from the group consisting of:

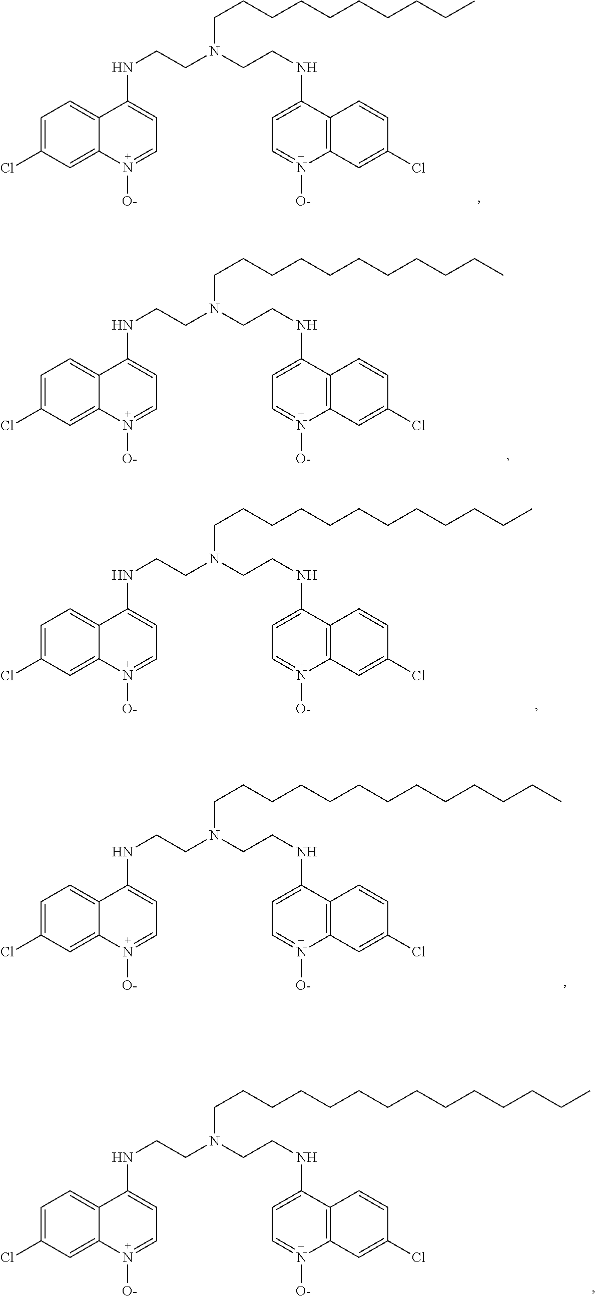

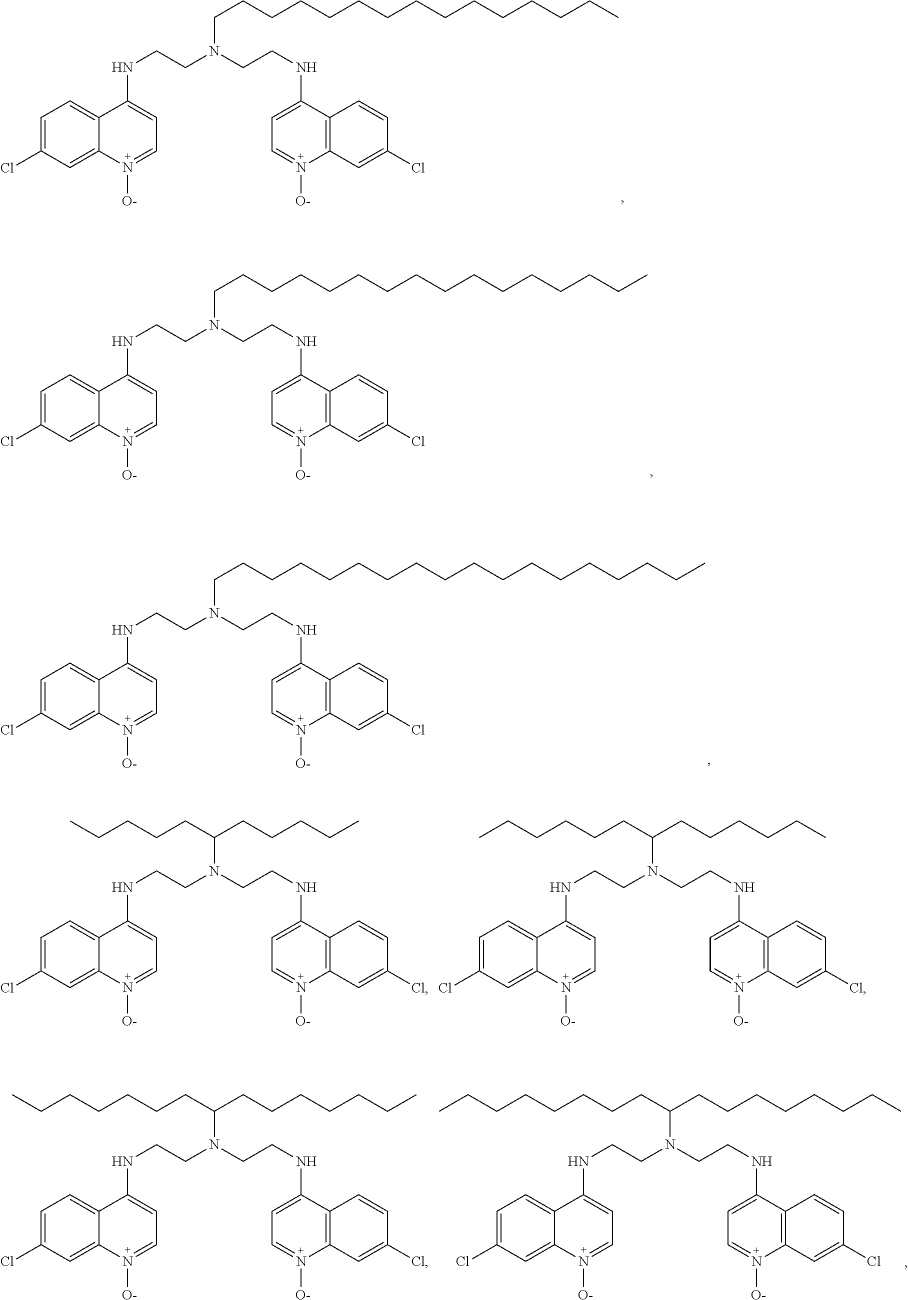

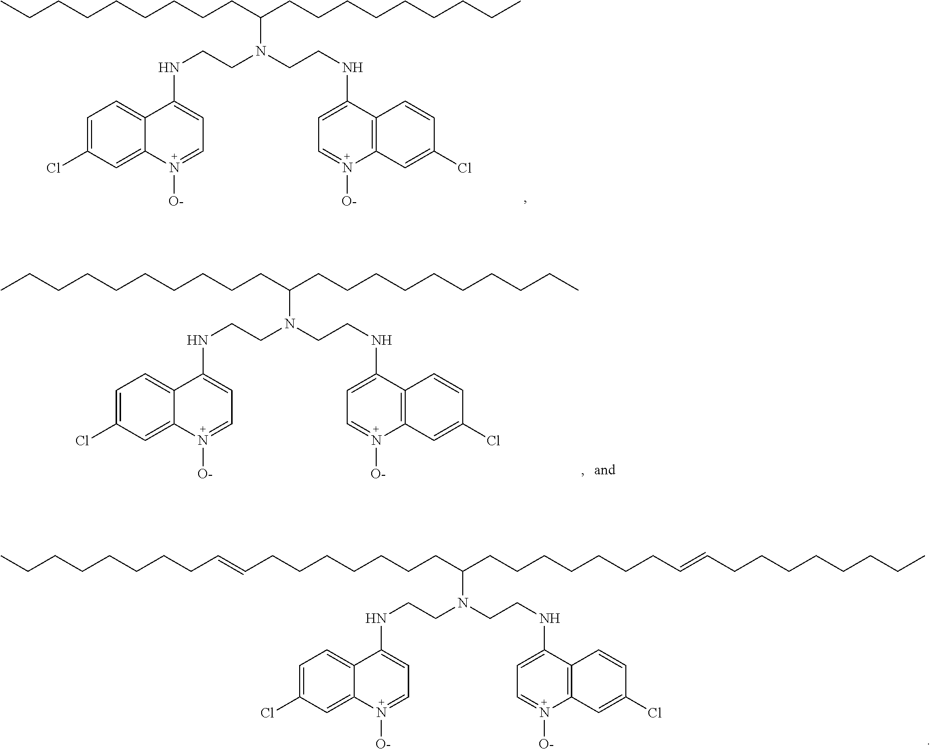

31. A nanocarrier having an interior and an exterior, the nanocarrier comprising a plurality of compounds of any one of claims 1 to 30, or a pharmaceutically acceptable salt thereof, wherein each compound self-assembles in an aqueous solvent to form the nanocarrier such that a hydrophobic pocket is formed in the interior of the nanocarrier, and a hydrophilic group self-assembles on the exterior of the nanocarrier.

32. The nanocarrier of claim 31, wherein the nanocarrier further comprises one or more hydrophobic drugs or imaging agents sequestered in the hydrophobic pocket of the nanocarrier.

33. The nanocarrier of claim 32, wherein the hydrophobic drug is a chemotherapeutic agent, a molecular targeted agent, an immunotherapeutic agent, a radiotherapeutic agent or a combination thereof.

34. The nanocarrier of claim 33, wherein the hydrophobic drug is the immunotherapeutic agent.

35. The nanocarrier of claim 33, wherein the hydrophobic drug is the radiotherapeutic agent.

36. The nanocarrier of claim 33, wherein the hydrophobic drug is the chemotherapeutic or molecular targeted agent.

37. The nanocarrier of claim 32 to 36, wherein the hydrophobic drug is a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR; INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated, estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10](pyro-Glu-His-Trp-Ser-Tyr-D-Ser(Bu t)-Leu-Arg-Pro-Azgly-NH2 acetate [C₅₉H₈₄N₁₈O₁₄—(C₂H₄O₂)X where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegfilgrastim, erythropoietin, epoetin alfa and darbepoetin alfa, ipilumumab, vemurafenib or a combination thereof.

38. The nanocarrier of any one of claims 31 to 37, wherein the nanocarrier comprises a plurality of compounds of any one of claims 24 to 30.

39. A method of treating a disease, comprising administering to a subject in need thereof, a therapeutically effective amount of a nanocarrier of any one of claims 31 to 38.

40. The method of claim 39, further comprising one or more additional agents, wherein the additional agent is a chemotherapeutic agent, a molecular targeted agent, an immunotherapeutic agent, a radiotherapeutic agent or a combination thereof.

41. The method of claim 40, wherein the additional agent is the immunotherapeutic agent.

42. The method of claim 40, wherein the additional agent is the radiotherapeutic agent.

43. The method of claim 40, wherein the additional agent is the chemotherapeutic or molecular targeted agent.

44. The method of any one of claims 40 to 43, wherein the additional agent is a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR; INO 1001, IPdR1 KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, irinotecan, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated, estrogen, bevacizumab, IMC-1C11, CHIR-258); 3-[5-(methylsulfonylpiperadinemethyl)-indolylj-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH2 acetate [C₅₉H₈₄N₁₈O₁₄—(C₂H₄O₂)X where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, irinotecan, topotecan, doxorubicin, docetaxel, vinorelbine, bevacizumab (monoclonal antibody) and erbitux, cremophor-free paclitaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, sspegfilgrastim, erythropoietin, epoetin alfa and darbepoetin alfa, ipilumumab, vemurafenib or a combination thereof.

45. The method of claim 39, wherein the disease is cancer.

46. The method of claim 45, wherein the cancer is bladder cancer, brain cancer, breast cancer, cervical cancer, cholangiocarcinoma, colorectal cancer, esophageal cancer, gall bladder cancer, gastric cancer, glioblastoma, intestinal cancer, head and neck cancer, leukemia, liver cancer, lung cancer, melanoma, myeloma, ovarian cancer, pancreatic cancer, prostate cancer and uterine cancer.

47. The method of claim 39, wherein the disease is coronavirus, malaria, antiphospholipid antibody syndrome, lupus, rheumatiod arthritis, chronic urticaria or Sjogren's disease.

48. The method of any one of claims 39 to 47, wherein the method of treating targets lysosomal disruption, lysosomal dysfunction and/or autophagy inhibition.

49. The method of any one of claim 39 or 48, wherein the method of treating targets the lysosome.

50. A method of imaging, comprising administering to a subject to be imaged, an effective amount of a nanocarrier of claims 31 to 38.
